SEQUENCE LISTING (94 sequences)

SEQ ID NO: 1 - DNA, 3 bp, Artificial Sequence
  Synthetic
  misc_feature (1)..(1): n is a, c, g, or t
  misc_feature (2)..(2): r is a, or g
nrg

SEQ ID NO: 2 - DNA, 3 bp, Artificial Sequence
  Synthetic
  misc_feature (1)..(1): n is a, c, g, or t
ngg

SEQ ID NO: 3 - DNA, 3 bp, Artificial Sequence
  Synthetic
  misc_feature (1)..(1): n is a, c, g, or t
nag

SEQ ID NO: 4 - DNA, 5 bp, Artificial Sequence
  Synthetic
  misc_feature (1)..(1): n is a, c, g, or t
  misc_feature (4)..(4): n is a, c, g, or t
nggng

SEQ ID NO: 5 - DNA, 7 bp, Artificial Sequence
  Synthetic
  misc_feature (1)..(2): n is a, c, g, or t
  misc_feature (7)..(7): w is a, or t
nnagaaw

SEQ ID NO: 6 - DNA, 4 bp, Artificial Sequence
  Synthetic
  misc_feature (1)..(1): n is a, c, g, or t
  misc_feature (4)..(4): r is a, or g
naar

SEQ ID NO: 7 - DNA, 5 bp, Artificial Sequence
  Synthetic
  misc_feature (1)..(2): n is a, c, g, or t
  misc_feature (4)..(5): r is a, or g
nngrr

SEQ ID NO: 8 - DNA, 6 bp, Artificial Sequence
  Synthetic
  misc_feature (1)..(2): n is a, c, g, or t
  misc_feature (4)..(5): r is a, or g
  misc_feature (6)..(6): n is a, c, g, or t
nngrrn

SEQ ID NO: 9 - DNA, 6 bp, Artificial Sequence
  Synthetic
  misc_feature (1)..(2): n is a, c, g, or t
  misc_feature (4)..(5): r is a, or g
nngrrt

SEQ ID NO: 10 - DNA, 6 bp, Artificial Sequence
  Synthetic
  misc_feature (1)..(2): n is a, c, g, or t
  misc_feature (4)..(5): r is a, or g
  misc_feature (6)..(6): v is a, c, or g
nngrrv

SEQ ID NO: 11 - DNA, 8 bp, Artificial Sequence
  Synthetic
  misc_feature (1)..(4): n is a, c, g, or t
nnnngatt

SEQ ID NO: 12 - DNA, 8 bp, Artificial Sequence
  Synthetic
  misc_feature (1)..(4): n is a, c, g, or t
  misc_feature (6)..(8): n is a, c, g, or t
nnnngnnn

SEQ ID NO: 13 - DNA, 3 bp, Artificial Sequence
  Synthetic
  misc_feature (1)..(1): n is a, c, g, or t
nga

SEQ ID NO: 14 - DNA, 6 bp, Artificial Sequence
  Synthetic
  misc_feature (1)..(3): n is a, c, g, or t
  misc_feature (4)..(5): r is a, or g
nnnrrt

SEQ ID NO: 15 - DNA, 6 bp, Artificial Sequence
  Synthetic
attcct

SEQ ID NO: 16 - DNA, 4 bp, Artificial Sequence
  Synthetic
  misc_feature (1)..(1): n is a, c, g, or t
  misc_feature (4)..(4): n is a, c, g, or t
ngan

SEQ ID NO: 17 - DNA, 4 bp, Artificial Sequence
  Synthetic
  misc_feature (1)..(1): n is a, c, g, or t
  misc_feature (3)..(3): n is a, c, g, or t
ngng

SEQ ID NO: 18 - protein, 1368 aa, Streptococcus pyogenes [sequence omitted]

SEQ ID NO: 19 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 20 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 21 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - DNA, 3159 bp, Artificial Sequence
  Synthetic
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 23 - DNA, 3159 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 24 - DNA, 4107 bp, Artificial Sequence
  Synthetic
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 25 - DNA, 3158 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 26 - DNA, 3159 bp, Artificial Sequence
  Synthetic
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 27 - DNA, 3159 bp, Artificial Sequence
  Synthetic
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 28 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 29 - DNA, 3242 bp, Artificial Sequence
  Synthetic
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 30 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 31 - DNA, 3156 bp, Artificial Sequence
  Synthetic
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 32 - DNA, 7009 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 33 - protein, 2414 aa, Artificial Sequence
  Synthetic
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 34 - protein, 617 aa, Artificial Sequence
  Synthetic
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 35 - protein, 1497 aa, Artificial Sequence
  Synthetic
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 36 - DNA, 4491 bp, Artificial Sequence
  Synthetic
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 37 - protein, 387 aa, Artificial Sequence
  Synthetic
MSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 38 - DNA, 1161 bp, Artificial Sequence
  Synthetic
atgtctgtggctttcgcggccccgaggcagcgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttaccagcaccacccacacagaatatgcctatgggtcctggagggatgaatcagagcggccctcccccacctccacgctctcacaacatgccttcagatggaatggtaggtgggggtcctcctgcaccgcacatgcagaaccagatgaacggccagatgcctgggcctaaccatatgcctatgcagggacctggacccaatcaactcaatatgacaaacagttccatgaatatgccttcaagtagccatggatccatgggaggttacaaccattctgtgccatcatcacagagcatgccagtacagaatcagatgacaatgagtcagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagggacagtatggaaattaccagcag

SEQ ID NO: 39 - protein, 378 aa, Artificial Sequence
  Synthetic
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 40 - DNA, 1134 bp, Artificial Sequence
  Synthetic
gatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 41 - protein, 522 aa, Artificial Sequence
  Synthetic
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 42 - DNA, 1566 bp, Artificial Sequence
  Synthetic
gatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 43 - DNA, 20 bp, Artificial Sequence
  Synthetic
tagtcttagagtatccagtg

SEQ ID NO: 44 - DNA, 20 bp, Artificial Sequence
  Synthetic
ggctagggatgaagaataaa

SEQ ID NO: 45 - DNA, 20 bp, Artificial Sequence
  Synthetic
aatatgtcacattctgtctc

SEQ ID NO: 46 - DNA, 20 bp, Artificial Sequence
  Synthetic
ggactatgggaggtcactaa

SEQ ID NO: 47 - DNA, 20 bp, Artificial Sequence
  Synthetic
gaaggttacacagaaccaga

SEQ ID NO: 48 - DNA, 20 bp, Artificial Sequence
  Synthetic
gccctgtaagcatcctgctg

SEQ ID NO: 49 - RNA, 20 nt, Artificial Sequence
  Synthetic
uagucuuagaguauccagug

SEQ ID NO: 50 - RNA, 20 nt, Artificial Sequence
  Synthetic
ggcuagggaugaagaauaaa

SEQ ID NO: 51 - RNA, 20 nt, Artificial Sequence
  Synthetic
aauaugucacauucugucuc

SEQ ID NO: 52 - RNA, 20 nt, Artificial Sequence
  Synthetic
ggacuaugggaggucacuaa

SEQ ID NO: 53 - RNA, 20 nt, Artificial Sequence
  Synthetic
gaagguuacacagaaccaga

SEQ ID NO: 54 - RNA, 20 nt, Artificial Sequence
  Synthetic
gcccuguaagcauccugcug

SEQ ID NO: 55 - protein, 5 aa, Artificial Sequence
  Synthetic
  REPEAT (1)..(5): repeat 0 to 10 times
GGGGS

SEQ ID NO: 56 - protein, 5 aa, Artificial Sequence
  Synthetic
GGGGG

SEQ ID NO: 57 - protein, 5 aa, Artificial Sequence
  Synthetic
GGAGG

SEQ ID NO: 58 - protein, 7 aa, Artificial Sequence
  Synthetic
GGGGSSS

SEQ ID NO: 59 - protein, 7 aa, Artificial Sequence
  Synthetic
GGGGAAA

SEQ ID NO: 60 - protein, 7 aa, Artificial Sequence
  Synthetic
PKKKRKV

SEQ ID NO: 61 - DNA, 86 bp, Artificial Sequence
  Synthetic
gtttaagagctatgctggaaacagcatagcaagtttaaataaggctagtccgttatcaacttgaaaaagtggcaccgagtcggtgc

SEQ ID NO: 62 - RNA, 86 nt, Artificial Sequence
  Synthetic
guuuaagagcuaugcuggaaacagcauagcaaguuuaaauaaggcuaguccguuaucaacuugaaaaaguggcaccgagucggugc

SEQ ID NO: 63 - DNA, 6813 bp, Artificial Sequence
  Synthetic
atggatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgctagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctccggctctggaggatctggcggctctagcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaactggactctggaggatccgactacaaagaccatgacggtgattataaagatcatgacatcgattacaaggatgacgatgacaagggaggatccaaggagaagagtgcttgtcctaaagatccagccaaacctccggccaaggcacaagttgtgggatggccaccggtgagatcataccggaagaacgtgatggtttcctgccaaaaatcaagcggtggcccggaggcggcggcgttcgtgaaggtatcaatggacggagcaccgtacttgaggaaaatcgatttgaggatgtataaaggcggatctggcggctctggaggatccagcatgtctgtggctttcgcggccccgaggcagcgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttaccagcaccacccacacagaatatgcctatgggtcctggagggatgaatcagagcggccctcccccacctccacgctctcacaacatgccttcagatggaatggtaggtgggggtcctcctgcaccgcacatgcagaaccagatgaacggccagatgcctgggcctaaccatatgcctatgcagggacctggacccaatcaactcaatatgacaaacagttccatgaatatgccttcaagtagccatggatccatgggaggttacaaccattctgtgccatcatcacagagcatgccagtacagaatcagatgacaatgagtcagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagggacagtatggaaattaccagcagtga

SEQ ID NO: 64 - protein, 2269 aa, Artificial Sequence
  Synthetic
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSGSGGSGGSSATMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKLDSGGSDYKDHDGDYKDHDIDYKDDDDKGGSKEKSACPKDPAKPPAKAQVVGWPPVRSYRKNVMVSCQKSSGGPEAAAFVKVSMDGAPYLRKIDLRMYKGGSGGSGGSSMSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 65 - DNA, 6549 bp, Artificial Sequence
  Synthetic
atggactacaaagaccatgacggtgattataaagatcatgacatcgattacaaggatgacgatgacaagcacgttgatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctccaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcatgtctgtggctttcgcggccccgaggcagcgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttaccagcaccacccacacagaatatgcctatgggtcctggagggatgaatcagagcggccctcccccacctccacgctctcacaacatgccttcagatggaatggtaggtgggggtcctcctgcaccgcacatgcagaaccagatgaacggccagatgcctgggcctaaccatatgcctatgcagggacctggacccaatcaactcaatatgacaaacagttccatgaatatgccttcaagtagccatggatccatgggaggttacaaccattctgtgccatcatcacagagcatgccagtacagaatcagatgacaatgagtcagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagggacagtatggaaattaccagcagtga

SEQ ID NO: 66 - protein, 2182 aa, Artificial Sequence
  Synthetic
MDYKDHDGDYKDHDIDYKDDDDKHVDALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASMSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 67 - DNA, 5274 bp, Artificial Sequence
  Synthetic
atggatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctccggctctggaggatctggcggctctagcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 68 - protein, 1757 aa, Artificial Sequence
  Synthetic
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSGSGGSGGSSATMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 69 - DNA, 5358 bp, Artificial Sequence
  Synthetic
atggactacaaagaccatgacggtgattataaagatcatgacatcgattacaaggatgacgatgacaagcacgttgatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctccaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgac

SEQ ID NO: 70 - protein, 1785 aa, Artificial Sequence
  Synthetic
DYKDHDGDYKDHDIDYKDDDDKHVDALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRAD

SEQ ID NO: 71 - DNA, 5616 bp, Artificial Sequence
  Synthetic
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaactggactctggaggatccgactacaaagaccatgacggtgattataaagatcatgacatcgattacaaggatgacgatgacaagggaggatccaaggagaagagtgcttgtcctaaagatccagccaaacctccggccaaggcacaagttgtgggatggccaccggtgagatcataccggaagaacgtgatggtttcctgccaaaaatcaagcggtggcccggaggcggcggcgttcgtgaaggtatcaatggacggagcaccgtacttgaggaaaatcgatttgaggatgtataaaggcggatctggcggctctggaggatccagcgatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgctagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctga

SEQ ID NO: 72 - protein, 1871 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKLDSGGSDYKDHDGDYKDHDIDYKDDDDKGGSKEKSACPKDPAKPPAKAQVVGWPPVRSYRKNVMVSCQKSSGGPEAAAFVKVSMDGAPYLRKIDLRMYKGGSGGSGGSSDALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 73 - DNA, 5385 bp, Artificial Sequence
  Synthetic
atggactacaaagaccatgacggtgattataaagatcatgacatcgattacaaggatgacgatgacaagcacgttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 74 - protein, 1794 aa, Artificial Sequence
  Synthetic
DYKDHDGDYKDHDIDYKDDDDKHVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 75 - DNA, 6786 bp, Artificial Sequence
  Synthetic
atggatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctccggctctggaggatctggcggctctagcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaactggactctggaggatccgactacaaagaccatgacggtgattataaagatcatgacatcgattacaaggatgacgatgacaagggaggatccaaggagaagagtgcttgtcctaaagatccagccaaacctccggccaaggcacaagttgtgggatggccaccggtgagatcataccggaagaacgtgatggtttcctgccaaaaatcaagcggtggcccggaggcggcggcgttcgtgaaggtatcaatggacggagcaccgtacttgaggaaaatcgatttgaggatgtataaaggcggatctggcggctctggaggatccagcgatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgctagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctga

SEQ ID NO: 76 - protein, 2260 aa, Artificial Sequence
  Synthetic
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSGSGGSGGSSATMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKLDSGGSDYKDHDGDYKDHDIDYKDDDDKGGSKEKSACPKDPAKPPAKAQVVGWPPVRSYRKNVMVSCQKSSGGPEAAAFVKVSMDGAPYLRKIDLRMYKGGSGGSGGSSDALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 77 - DNA, 6045 bp, Artificial Sequence
  Synthetic
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaactggactctggaggatccgactacaaagaccatgacggtgattataaagatcatgacatcgattacaaggatgacgatgacaagggaggatccaaggagaagagtgcttgtcctaaagatccagccaaacctccggccaaggcacaagttgtgggatggccaccggtgagatcataccggaagaacgtgatggtttcctgccaaaaatcaagcggtggcccggaggcggcggcgttcgtgaaggtatcaatggacggagcaccgtacttgaggaaaatcgatttgaggatgtataaaggcggatctggcggctctggaggatccgatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttga

SEQ ID NO: 78 - protein, 2014 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKLDSGGSDYKDHDGDYKDHDIDYKDDDDKGGSKEKSACPKDPAKPPAKAQVVGWPPVRSYRKNVMVSCQKSSGGPEAAAFVKVSMDGAPYLRKIDLRMYKGGSGGSGGSDALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 79 - DNA, 6330 bp, Artificial Sequence
  Synthetic
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaactggactctggaggatccgactacaaagaccatgacggtgattataaagatcatgacatcgattacaaggatgacgatgacaagggaggatccaaggagaagagtgcttgtcctaaagatccagccaaacctccggccaaggcacaagttgtgggatggccaccggtgagatcataccggaagaacgtgatggtttcctgccaaaaatcaagcggtggcccggaggcggcggcgttcgtgaaggtatcaatggacggagcaccgtacttgaggaaaatcgatttgaggatgtataaaggcggatctggcggctctggaggatccattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggactga

SEQ ID NO: 80 - protein, 2109 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKLDSGGSDYKDHDGDYKDHDIDYKDDDDKGGSKEKSACPKDPAKPPAKAQVVGWPPVRSYRKNVMVSCQKSSGGPEAAAFVKVSMDGAPYLRKIDLRMYKGGSGGSGGSIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 81 - DNA, 5994 bp, Artificial Sequence
  Synthetic
atggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 82 - protein, 1998 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 83 - DNA, 5994 bp, Artificial Sequence
  Synthetic
atgggtattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggacggctctggaggatctggcggctctagcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 84 - protein, 1998 aa, Artificial Sequence
  Synthetic
MGIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDGSGGSGGSSATMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 85 - DNA, 6075 bp, Artificial Sequence
  Synthetic
atggactacaaagaccatgacggtgattataaagatcatgacatcgattacaaggatgacgatgacaagcacgttattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggacaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgac

SEQ ID NO: 86 - protein, 2024 aa, Artificial Sequence
  Synthetic
DYKDHDGDYKDHDIDYKDDDDKHVIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRAD

SEQ ID NO: 87 - DNA, 5643 bp, Artificial Sequence
  Synthetic
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaactggactctggaggatccgactacaaagaccatgacggtgattataaagatcatgacatcgattacaaggatgacgatgacaagggaggatccaaggagaagagtgcttgtcctaaagatccagccaaacctccggccaaggcacaagttgtgggatggccaccggtgagatcataccggaagaacgtgatggtttcctgccaaaaatcaagcggtggcccggaggcggcggcgttcgtgaaggtatcaatggacggagcaccgtacttgaggaaaatcgatttgaggatgtataaaggcggatctggcggctctggaggatccagcatgtctgtggctttcgcggccccgaggcagcgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttaccagcaccacccacacagaatatgcctatgggtcctggagggatgaatcagagcggccctcccccacctccacgctctcacaacatgccttcagatggaatggtaggtgggggtcctcctgcaccgcacatgcagaaccagatgaacggccagatgcctgggcctaaccatatgcctatgcagggacctggacccaatcaactcaatatgacaaacagttccatgaatatgccttcaagtagccatggatccatgggaggttacaaccattctgtgccatcatcacagagcatgccagtacagaatcagatgacaatgagtcagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagggacagtatggaaattaccagcagtga

SEQ ID NO: 88 - protein, 1880 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKLDSGGSDYKDHDGDYKDHDIDYKDDDDKGGSKEKSACPKDPAKPPAKAQVVGWPPVRSYRKNVMVSCQKSSGGPEAAAFVKVSMDGAPYLRKIDLRMYKGGSGGSGGSSMSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 89 - DNA, 5304 bp, Artificial Sequence
  Synthetic
atggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcatgtctgtggctttcgcggccccgaggcagcgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttaccagcaccacccacacagaatatgcctatgggtcctggagggatgaatcagagcggccctcccccacctccacgctctcacaacatgccttcagatggaatggtaggtgggggtcctcctgcaccgcacatgcagaaccagatgaacggccagatgcctgggcctaaccatatgcctatgcagggacctggacccaatcaactcaatatgacaaacagttccatgaatatgccttcaagtagccatggatccatgggaggttacaaccattctgtgccatcatcacagagcatgccagtacagaatcagatgacaatgagtcagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagggacagtatggaaattaccagcag

SEQ ID NO: 90 - protein, 1768 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASMSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 91 - protein, 51 aa, Artificial Sequence
  Synthetic
RADALDDFDLDMLGSDALDDFDLDMLGSDALDDFDLDMLGSDALDDFDLDM

SEQ ID NO: 92 - DNA, 153 bp, Artificial Sequence
  Synthetic
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatg

SEQ ID NO: 93 - protein, 718 aa, Artificial Sequence
  Synthetic
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 94 - DNA, 2154 bp, Artificial Sequence
  Synthetic
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

(19) United States
(12) Patent Application Publication
(10) Pub. No.: US 2023/0159927 A1
(43) Pub. Date: May 25, 2023

(54) CHROMATIN REMODELERS TO ENHANCE TARGETED GENE ACTIVATION

(71) Applicant: Duke University, Durham, NC (US)

(72) Inventors: Charles A. Gersbach, Chapel Hill, NC (US); Nahid Iglesias, Durham, NC (US)

(21) Appl. No.: 17/922,751

(22) PCT Filed: May 7, 2021
(86) PCT No.: PCT/US2021/031436

§ 371 Date: Nov. 1, 2022

Related U.S. Application Data
(60) Provisional application No. 63/022,174, filed on May 8, 2020.
(60) Provisional application No. 63/094,158, filed on Oct. 20, 2020.

(51) Int. Cl. C12N 15/113; C12N 9/22

(52) U.S. Cl. CPC C12N 15/113; C12N 9/22; C12N 2310/20

(57) ABSTRACT

Disclosed herein are fusion proteins for the targeted activation of genes as well as compositions and methods and DMA Targeting Systems comprising the same. The fusion protein may include at least one first polypeptide domain and at least one second polypeptide domain. The first polypeptide domain includes a DMA binding protein, such as a zinc finger protein, a TALE, or a Cas protein, that targets the fusion protein for binding to a specific DNA sequence. The second polypeptide domain includes a modulator of chromatin structure. The fusion protein may further include a third polypeptide domain, the third polypeptide domain including a transcriptional activator domain.

### CROSS-REFERENCE TO RELATED APPLICATIONS

This application claims priority to U.S. Provisional Patent Application No. 63/022,174, filed May 8, 2020, and U.S. Provisional Patent Application No. 63/094,158 d Oct. 20, 2020, each of which is incorporated herein by reference in its entirety.

### STATEMENT REGARDING FEDERALLY SPONSORED RESEARCH

This invention was made with government support under grant EFMA-1830957 awarded by the National Science Foundation, grant SP5243390 awarded by the Defense Advanced Research Projects Agency, and grant U01Al146356 awarded by the National Institutes of Health. The government has certain rights in the invention.

### FIELD

This disclosure relates to compositions and methods for the targeted activation of genes.

### INTRODUCTION

Targeted activation of endogenous genes with synthetic transcription factors or epigenome editors made from DNA-targeting systems such as zinc finger proteins, TALEs, and CRISPR-Cas systems, are broadly useful for gene therapy, regenerative medicine, and programming stem cell differentiation. However, in some cases the potency or specificity of gene activation is insufficient to generate the desired phenotype or biological effect. There is a need for improved systems for activating expression of a specific gene.

### SUMMARY

In an aspect, the disclosure relates to a fusion protein comprising at least two heterologous polypeptide domains, wherein the first polypeptide domain comprises a DNA binding protein and the second polypeptide domain comprises a modulator of chromatin structure. In some embodiments, the fusion protein further comprises a third polypeptide domain. In some embodiments, the first polypeptide domain comprises a CRISPR-associated (Cas) protein, a TALE, or a zinc finger protein. In some embodiments, the Cas protein comprises at least one amino acid mutation that eliminates nuclease activity of the Cas protein. In some embodiments, the Cas protein comprises a Cas9 protein. In some embodiments, the Cas9 protein is nuclease-deficient dCas9 and comprises a polypeptide having at least 75% sequence identity to SEQ ID NO: 20 or 21 or is encoded by a polynucleotide comprising a sequence having at least 75% identity to SEQ ID NO: 22 or 23. In some embodiments, the modulator of chromatin structure comprises a nucleosome rearranging protein. In some embodiments, the modulator of chromatin structure comprises the SS18 subunit of the BAF chromatin remodeling complex or a fragment thereof or a variant thereof. In some embodiments, the SS18 subunit comprises a polypeptide having at least 75% sequence identity to SEQ ID NO: 37. In some embodiments, the third polypeptide domain comprises a transcriptional activator domain. In some embodiments, the transcriptional activator domain comprises VP64, VPH, VPR, p65, TET1, or p300, or a combination thereof or a fragment thereof or a variant thereof. In some embodiments, the VP64 comprises a polypeptide having at least 75% sequence identity to SEQ ID NO: 91. In some embodiments, the TET1 comprises a polypeptide having at least 75% sequence identity to SEQ ID NO: 93. In some embodiments, the VPH comprises a polypeptide having at least 75% sequence identity to SEQ ID NO: 39, In some embodiments, the VPR comprises a polypeptide having at least 75% sequence identity to SEQ ID NO: 41. In some embodiments, the p300 comprises a polypeptide having at least 75% sequence identity to SEQ ID NO: 33 or 34. In some embodiments, the fusion protein comprises one or more second polypeptide domain(s). In some embodiments, the one or more second polypeptide domain(s) is fused to the C-terminus or the N-terminus of the first polypeptide domain, or a combination thereof. In some embodiments, the N-terminus of the second polypeptide is operably linked to the C-terminus of the first polypeptide domain, or the C-terminus of the second polypeptide is operably linked to the N-terminus of the first polypeptide domain. In some embodiments, the fusion protein comprises one or more third polypeptide domain(s). In some embodiments, the one or more third polypeptide domain is fused to the C-terminus or the N-terminus of the first polypeptide domain, or a combination thereof. In some embodiments, the N-terminus of the third polypeptide is operably linked to the C-terminus of the first polypeptide domain, or the C-terminus of the third polypeptide is operably linked to the N-terminus of the first polypeptide domain. In some embodiments, the first polypeptide domain comprises dCas9, the second polypeptide domain comprises SS18, and the third polypeptide domain comprises VPH. In some embodiments, the fusion protein comprises VPH-dCas9-SS18 or SS18-dCas9-VPH or variants thereof. In some embodiments, the fusion protein comprises a polypeptide having at least 75% sequence identity to SEQ ID NO: 64 or 66. In some embodiments, the first polypeptide domain comprises dCas9, the second polypeptide domain comprises SS18, and the third polypeptide domain comprises VPR. In some embodiments, the fusion protein comprises VPR-dCas9-SS18 or SS18-dCas9-VPR or variants thereof. In some embodiments, the first polypeptide domain comprises dCas9, the second polypeptide domain comprises SS18, and the third polypeptide domain comprises p300, In some embodiments, the fusion protein comprises p300-dCas9-SS18 or SS18-dCas9-p300 or variants thereof. In some embodiments, the first polypeptide domain comprises dCas9, the second polypeptide domain comprises SS18, and the third polypeptide domain comprises VP64. In some embodiments, the fusion protein comprises VP64-dCas9-SS18 or SS18-dCas9-VP64 or variants thereof. In some embodiments, the fusion protein activates transcription of a target gene. In some embodiments, the fusion protein increases the level of mRNA expression of a target gene in a cell containing the fusion protein relative to a control. In some embodiments, the level of mRNA expression of the target gene is increased at least 5-fold, at least 50-fold, at least 100-fold, at least 1,000-fold, at least 10,000-fold, or at least 20,000-fold relative to a control. In some embodiments, the level of mRNA expression of the target gene is increased by 5-fold to 10,000-fold, 5-fold to 30,000-fold, 5-fold to 50,000-fold, 5-fold to 100,000-fold, 10,000-fold to 30,000-fold, 20,000-fold to 30,000-fold, 15,000-fold to 25,000-fold, 1,000-fold to 50,000-fold, or 1.000-fold to 100,000-fold relative to a control. In some embodiments, the control is the level of mRNA expression of the target gene in a cell not containing the fusion protein. In some embodiments, the target gene is gamma globin genes 1 and 2 (HBG1/2).

In a further aspect, the disclosure relates to DNA Targeting System. The DNA Targeting System may include (a) a fusion protein as detailed herein, wherein the first polypeptide domain comprises a zinc finger protein or a TALE; or (b) a gRNA and a fusion protein as detailed herein, wherein the first polypeptide domain comprises a Cas protein, and wherein the gRNA targets a target gene. In some embodiments, gRNA targets a regulatory region of the target gene. In some embodiments, the regulatory region is a promoter sequence of the target gene. Another aspect of the disclosure provides DNA Targeting System comprising a gRNA that recruits a modulator of chromatin structure to a target sequence. In some embodiments, the modulator of chromatin structure comprises the SS18 subunit of the BAF chromatin remodeling complex. In some embodiments, the gRNA is encoded by or binds to a target sequence selected from SEQ ID NOs: 43-48, a complement thereof, a truncation thereof, or a variant thereof, or the gRNA is encoded by or binds to a target sequence having at least 70% sequence identity to a sequence selected from SEQ ID NOs: 43-48, a complement thereof, a truncation thereof, or a variant thereof. In some embodiments, the gRNA comprises a polynucleotide sequence selected from SEQ ID NOs: 49-54, a complement thereof, a truncation thereof, or a variant thereof, or the gRNA comprises a polynucleotide having at least 70% sequence identity to a sequence selected from SEQ ID NOs: 49-54, a complement thereof, a truncation thereof, or a variant thereof.

Another aspect of the disclosure provides a method of increasing expression of a target gene in a cell. The method may include contacting the cell with a fusion protein as detailed herein or a DNA Targeting system as detailed herein. In some embodiments, the target gene is gamma globin genes 1 and 2 (HBG1/2).

Another aspect of the disclosure provides a gRNA encoded by or binding to a target sequence selected from SEQ ID NOs: 43-48, a complement thereof, a truncation thereof, or a variant thereof, or comprising a polynucleotide sequence selected from SEQ ID NOs: 49-54, a complement thereof, a truncation thereof, or a variant thereof.

The disclosure provides for other aspects and embodiments that will be apparent in light of the following detailed description and accompanying figures.

### BRIEF DESCRIPTION OF THE DRAWINGS

FIG. 1A shows the effect of indicated dCas9 activator fusions on HBG112 expression when targeted to the HBG1/2 promoter (with 2 gRNAs) or to the distal HS2 enhancer of the globin locus (with 4 gRNAs) in HEK293T cells, evaluated by RT-qPCR (n=2). FIG. 1B shows a schematic of the indicated dCas9 activator fusions (NLS, nuclear localization signal). FIG. 1C is a Western blot showing that the expression of the indicated dCas9 fusions is similar. Asterisk denotes degradation products.

FIG. 2 shows the effect of indicated dCas9 activator fusions on HBG1/2 expression when targeted to the HBG112 promoter (with 2 gRNAs) in HEK293T cells, evaluated by RT-qPCR (n=2).

FIG. 3 shows the effect of indicated dCas9 activator fusions on HBG1/2 expression when targeted to the HBG1/2 promoter (with 2 gRNAs) (left) and the expression level of the dCas9 activator fusions (right) in HEK293T cells, evaluated by RT-qPCR (n=2).

FIG. 4 shows the effect of indicated dCas9 activator fusions on HBG1/2 expression when targeted to the HBG1/2 promoter (with 2 gRNAs) (left) and the expression level of the dCas9 activator fusions (right) in HEK293T cells, evaluated by RT-qPCR (n=2).

### DETAILED DESCRIPTION

Described herein are fusion proteins for the targeted activation of genes as well as compositions and methods comprising the same. As detailed herein, it was demonstrated that combining modulators of chromatin structure, such as proteins that rearrange nucleosomes and/or cause movement of DNA in relation to the nucleosomes, with activator domains can lead to more potent gene activation in human cells. Remodelers of chromatin structure can cooperate with co-recruited transcriptional activation domains to more robustly activate target gene expression. The fusion protein may include at least two heterologous polypeptide domains. The first polypeptide domain includes a DNA binding protein, such as a zinc finger protein, a TALE, or a Cas9 protein, that targets the fusion protein for binding to a specific DNA sequence. The second polypeptide domain includes a modulator of chromatin structure, such as a nucleosome rearranging domain. The fusion protein may further include a third polypeptide domain, the third polypeptide domain including a transcriptional activator domain. The fusion protein may be incorporated into a DNA Targeting System and may be used to activate expression of a target gene in a cell.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a target region that is recognized and bound by the CRISPR/Cas-based gene editing system.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group, ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice. A control may be a subject or cell without a fusion protein as detailed herein, A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construe” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Genome editing” or “gene editing” as used herein refers to changing a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to a nucleic acid or protein comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible. “Nuclease mediated NHEJ” as used herein refers to NHEJ that is initiated after a nuclease cuts double stranded DNA.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand, Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribs-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. Promoters that target muscle-specific stem cells may include the CK8 promoter, the Spc5-12 promoter, and the MHCK7 promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

“Substantially identical” can mean that a first and second amino acid or polynucleotide sequence have at least 60%, 65%, 70%, 75%, 80%, 85%, 90%, 95%, 96%, 97%, 98%, or 99% sequence identity over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated. In certain embodiments, the target gene is a gamma globin gene.

“Target region” as used herein refers to the region of the target gene to which the CRISPR/Cas9-based gene editing or targeting system is designed to bind.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Transcriptional regulatory elements” or “regulatory elements” refers to a genetic element which can control the expression of nucleic acid sequences, such as activate, enhance, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “treatment” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

“Variant” with respect to a peptide or polypeptide refers to a peptide or polypeptide that differs in amino acid sequence by the insertion, deletion, conservative substitution, or non-conservative substitution of amino acids, but retains at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide, to promote an immune response, to activate or increase transcription, to bind or target a polynucleotide or a polypeptide, rearrange or remodel chromatin, or to catalyze a reaction such as demethylation or acetylation. Variant can mean a functional fragment or truncation thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector, Viral vectors include, but are not limited to, adenovirus vector, adeno-associated virus (AAV) vector, retrovirus vector, or lentivirus vector. A vector may be an adeno-associated virus (AAV) vector. The vector may encode a Cas9 protein or fusion protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Fusion Protein

Provided herein is a fusion protein. The fusion protein may activate transcription of a target gene. The fusion protein may increase transcription or expression of a target gene by at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 90%, at least about 95%, at least about 200%, at least about 300%, at least about 400%, or at least about 500% relative to a control. The fusion protein may increase transcription or expression of a target gene by less than 1,000,000-fold, less than 500,000-fold, less than 100,000-fold, less than 10,000-fold, less than 1,000-fold, less than 100-fold, 10-fold, less than 5-fold, less than 4-fold, less than 3-fold, or less than 2-fold relative to a control. The control may be, for example, transcription or expression of the target gene in a cell in which the fusion protein was not introduced.

Activation of transcription or expression of a target gene may include an increase in the level of mRNA expression from the target gene, relative to a control. The control may be, for example, the level of mRNA expression of the target gene in a cell lacking the fusion protein. The mRNA expression level from the target gene may be increased at least 2-fold, at least 3-fold, at least 4-fold, at least 5-fold, at least 10-fold, at least 20-fold, at least 30-fold, at least 40-fold, at least 50-fold, at least 100-fold, at least 500-fold, at least 1,000-fold, at least 5,000-fold, at least 10,000-fold, at least 15,000-fold, at least 20,000-fold, at least 25,000-fold, at least 30,000-fold, at least 50,000-fold, or at least 100,000-fold relative to a control. The mRNA expression level from the target gene may be increased less than 1,000,000-fold, less than 500,000-fold, less than 100,000-fold, less than 50,000-fold, less than 40,000-fold, less than 30,000-fold, less than 25,000-fold, less than 20,000-fold, less than 15,000-fold, less than 10,000-fold, less than 5,000-fold, less than 1,000-fold, less than 500-fold, less than 100-fold, less than 50-fold, or less than 10-fold relative to a control. The mRNA expression level from the target gene may be increased 2-fold to 50-fold, 2-fold to 100-fold, 2-fold to 500-fold, 2-fold to 1,000-fold, 2-fold to 5,000-fold, 2-fold to 10,000-fold, 2-fold to 15,000-fold, 2-fold to 20,000-fold, 2-fold to 25,000-fold, 2-fold to 30,000-fold, 2-fold to 50,000-fold, 2-fold to 100,000-fold, 2-fold to 500,000-fold, 2-fold to 1,000,000-fold, 10-fold to 50-fold, 10-fold to 100-fold, 10-fold to 500-fold, 10-fold to 1,000-fold, 10-fold to 5,000-fold, 10-fold to 10,000-fold, 10-fold to 15,000-fold, 10-fold to 20,000-fold, 10-fold to 25,000-fold, 10-fold to 30,000-fold, 10-fold to 50,000-fold, 10-fold to 100,000-fold, 10-fold to 500,000-fold, 10-fold to 1,000,000-fold, 100-fold to 500-fold, 100-fold to 1,000-fold, 100-fold to 5,000-fold, 100-fold to 10,000-fold, 100-fold to 15,000-fold, 100-fold to 20,000-fold, 100-fold to 25,000-fold, 100-fold to 30,000-fold, 100-fold to 50,000-fold, 100-fold to 100,000-fold, 100-fold to 500,000-fold, 100-fold to 1,000,000-fold, 1,000-fold to 5,000-fold, 1,000-fold to 10,000-fold, 1,000-fold to 15,000-fold, 1,000-fold to 20,000-fold, 1,000-fold to 25,000-fold, 1,000-fold to 30,000-fold, 1,000-fold to 50,000-fold, 1,000-fold to 100,000-fold, 1,000-fold to 500,000-fold, or 1,000-fold to 1,000,000-fold relative to a control.

Activation of transcription or expression of a target gene may include an increase in the level of protein expressed from the target gene, relative to a control. The control may be, for example, the level of protein expressed from the target gene in a cell lacking the fusion protein. The level of protein expression from the target gene may be increased at least 2-fold, at least 3-fold, at least 4-fold, at least 5-fold, at least 10-fold, at least 20-fold, at least 30-fold, at least 40-fold, at least 50-fold, at least 100-fold, at least 500-fold, at least 1,000-fold, at least 5,000-fold, at least 10,000-fold, at least 15,000-fold, at least 20,000-fold, at least 25,000-fold, at least 30,000-fold, at least 50,000-fold, or at least 100,000-fold relative to a control. The level of protein expression from the target gene may be increased less than 1,000,000-fold, less than 500,000-fold, less than 100,000-fold, less than 50,000-fold, less than 40,000-fold, less than 30,000-fold, less than 25,000-fold, less than 20,000-fold, less than 15,000-fold, less than 10,000-fold, less than 5,000-fold, less than 1,000-fold, less than 500-fold, less than 100-fold, less than 50-fold, or less than 10-fold relative to a control. The level of protein expression from the target gene may be increased 2-fold to 50-fold, 2-fold to 100-fold, 2-fold to 500-fold, 2-fold to 1,000-fold, 2-fold to 5,000-fold, 2-fold to 10,000-fold, 2-fold to 15,000-fold, 2-fold to 20,000-fold, 2-fold to 25,000-fold, 2-fold to 30,000-fold, 2-fold to 50,000-fold, 2-fold to 100,000-fold, 2-fold to 500,000-fold, 2-fold to 1,000,000-fold, 10-fold to 50-fold, 10-fold to 100-fold, 10-fold to 500-fold, 10-fold to 1,000-fold, 10-fold to 5,000-fold, 10-fold to 10,000-fold, 10-fold to 15,000-fold, 10-fold to 20,000-fold, 10-fold to 25,000-fold, 10-fold to 30,000-fold, 10-fold to 50,000-fold, 10-fold to 100,000-fold, 10-fold to 500,000-fold, 10-fold to 1,000,000-fold, 100-fold to 500-fold, 100-fold to 1,000-fold, 100-fold to 5,000-fold, 100-fold to 10,000-fold, 100-fold to 15,000-fold, 100-fold to 20,000-fold, 100-fold to 25,000-fold, 100-fold to 30,000-fold, 100-fold to 50,000-fold, 100-fold to 100,000-fold, 100-fold to 500,000-fold, 100-fold to 1,000,000-fold, 1,000-fold to 5,000-fold, 1,000-fold to 10,000-fold, 1,000-fold to 15,000-fold, 1,000-fold to 20,000-fold, 1,000-fold to 25,000-fold, 1,000-fold to 30,000-fold, 1,000-fold to 50,000-fold, 1,000-fold to 100,000-fold, 1,000-fold to 500,000-fold, or 1,000-fold to 1,000,000-fold relative to a control.

The fusion protein comprises at least two heterologous polypeptide domains. The first polypeptide domain comprises a DNA binding protein. The first polypeptide domain is fused to at least one second polypeptide domain. The second polypeptide domain comprises a modulator of chromatin structure. In some embodiments, the fusion protein further includes at least one third polypeptide domain. The third polypeptide domain comprises a transcriptional activator domain.

The linkage to the first polypeptide domain, to the second polypeptide domain, and/or to the third polypeptide domain can be through reversible or irreversible covalent linkage or through a non-covalent linkage, as long as the linker does not interfere with the function of the first, second, or third polypeptide domain(s). For example, a DNA binding protein can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione 5-transferase (GST)-glutathione interaction. As yet another example, they can be linked covalently but reversibly with linkers such as dibromomaleimide (DBM) or amino-thiol conjugation.

In some embodiments, the fusion protein further includes at least one linker. A linker may be included anywhere in the polypeptide sequence of the fusion protein, for example, between the first and second domains, between the first and third domains, and/or between the second and third domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser)ₙ, wherein n is an integer between 0 and 10 (SEQ ID NO: 55). In a GS linker, n can be adjusted to optimize the linker length and achieve appropriate separation of the functional domains. Other examples of linkers may include, for example, Gly-Gly-Gly-Gly-Gly (SEQ ID NO: 56), Gly-Gly-Ala-Gly-Gly (SEQ ID NO: 57), Gly/Ser rich linkers such as Gly-Gly-Gly-Gly-Ser-Ser-Ser (SEQ ID NO: 58), or Gly/Ala rich linkers such as Gly-Gly-Gly-Gly-Ala-Ala-Ala (SEQ ID NO: 59).

In some embodiments, the fusion protein includes a nuclear localization sequence (NLS). Nuclear localization sequences are known in the art. Nuclear localization sequences include, for example, SV40 NLS (Pro-Lys-Lys-Lys-Arg-Lys-Val: SEQ ID NO: 60).

a. First Domain: DNA Binding Protein

The first polypeptide domain of the fusion protein comprises a DNA binding protein. The DNA binding protein may be a zinc finger protein, a transcription activator-like effector (TALE), or a Cas protein. The DNA binding protein targets the fusion protein for binding to a specific DNA sequence.

Alternative to a DNA binding protein, or in addition to, the fusion protein may include an aptamer. Aptamers are polynucleotides or polypeptides that specifically recognize and bind to a specific target molecule, such as to a DNA sequence.

i) Zinc Finger Protein

The DNA binding protein may comprise a zinc finger protein. A zinc finger protein is a protein that includes one or more zinc finger domains. Zinc finger domains are relatively small protein motifs that contain multiple finger-like protrusions that make tandem contacts with their target molecule such as a DNA target molecule. A zinc finger domain may bind one or more zinc ions or other metal ion such as iron, or in some cases a zinc finger domain forms salt bridges to stabilize the finger-like folds. The zinc binding portion of a zinc finger protein may include one or more cysteine residues and/or one or more histidine residues to coordinate the zinc or other metal ion. A zinc finger protein recognizes and binds to a particular DNA sequence via the zinc finger domain.

ii) TALE

The DNA binding protein may comprise a transcription activator-like effector (TALE). A TALE is another type of protein that recognizes and binds to a particular DNA sequence. The DNA-binding domain of a TALE includes an array of tandem 33-35 amino acid repeats, also known as RVD modules. Each RVD module specifically recognizes a single base pair of DNA. RVD modules may be arranged in any order to assemble an array that recognizes a defined DNA sequence. The binding specificity of a TALE DNA-binding domain is determined by the RVD array followed by a single truncated repeat of, for example, 20 amino acids. A TALE DNA-binding domain may have an array of 12 to 27 RVD modules, each RVD module recognizing a single base pair of DNA. Specific RVDs have been identified that recognize each of the four possible DNA nucleotides (A, T, C, and G). Because the TALE DNA-binding domains are modular, repeats that recognize the four different DNA nucleotides may be linked together to recognize any particular DNA sequence. These targeted DNA-binding domains may then be combined with catalytic domains to create functional enzymes, including artificial transcription factors and/or nucleases. In some embodiments, the TALE specifically binds to a target sequence associated with a target gene.

iii) Cas Protein

The DNA binding protein may include a Cas protein or a mutated Cas protein. “Clustered Regularly interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas proteins include, for example, Cas9, Cas12, and Cas12a. In some embodiments, the Cas protein is a Cas9 protein, Cas9 forms a complex with the 3′ end of the sgRNA (which may be referred interchangeably herein as “gRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 by recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Cas protein may be from any of the Types I, II, and III effector systems. The Type II effector system carries out targeted DNA double-strand break in four sequential steps, using a single effector enzyme, Cas9, to cleave dsDNA. Compared to the Type I and Type ill effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarily between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type 11 systems have differing PAM requirements.

An engineered form of the Type H effector system of Streptococcus pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneurnoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobiurn sp., Brevibacilius laterosporus, Campylobacter coli, Campylobacter jejuni, Campylobacter lari, Candidatus Puniceispirillum, Clostridium cellulolyticum, Clostridium perfringens, Corynebacterium accolens, Corynebacteriurn diphtheria, Corynebacterium matruchotii, Dinomseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter musteiae, ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 18. In certain embodiments, the Cas9 molecule is a Staphylococcus aureus Cas9 molecule (also referred herein as “SaCas9”). SaCas9 may comprise an amino acid sequence of SEQ ID NO: 19.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic add sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi:10.1038/nmeth.2881). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G; SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G; SEQ ID NO: 7), NNGRRN (R=A or G; SEQ ID NO: 8), NNGRRT (R=A or G; SEQ ID NO: 9), or NNGRRV (R=A or G; SEQ ID NO: 10). In the aforementioned embodiments, N can be any nucleotide residue, for example, any of A, G, C, or T.

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A, A S. pyogenes Cas9 protein with the D10A mutation may comprise an amino acid sequence of SEQ ID NO: 20. In some embodiments, the dCas9 protein comprises a polypeptide having at least 75%, at least 80%, at least 85%, at least 90%, at least 95%, at least 98%, at least 99%, or 100% sequence identity to SEQ ID NO: 20, or any range between any two of these values. A S. pyogenes Cas9 protein with D10A and H849A mutations may comprise an amino acid sequence of SEQ ID NO: 21. In some embodiments, the dCas9 protein comprises a polypeptide having at least 75%, at least 80%, at least 85%, at least 90%, at least 95%, at least 98%, at least 99%, or 100% sequence identity to SEQ ID NO: 21, or any range between any two of these values. Exemplary mutations with reference to the S. aureus Cas9 sequence to inactivate the nuclease activity include Di OA and N580A. In certain embodiments, the mutant S. aureus Cas9 molecule comprises a D10A mutation. The nucleotide sequence encoding this mutant S. aureus Cas9 is set forth in SEQ ID NO: 22. In some embodiments, the dCas9 protein comprises a polypeptide encoded by a polynucleotide having at least 75%, at least 80%, at least 85%, at least 90%, at least 95%, at least 98%, at least 99%, or 100% sequence identity to SEQ ID NO: 22, or any range between any two of these values. In certain embodiments, the mutant S. aureus Cas9 molecule comprises a N580A mutation. The nucleotide sequence encoding this mutant S. aureus Cas9 molecule is set forth in SEQ ID NO: 23. In some embodiments, the dCas9 protein comprises a polypeptide encoded by a polynucleotide having at least 75%, at least 80%, at least 85%, at least 90%, at least 95%, at least 98%, at least 99%, or 100% sequence identity to SEQ ID NO: 23, or any range between any two of these values.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 24. Exemplary codon optimized nucleic acid sequences encoding a Cas9 molecule of S. aureus, and optionally containing nuclear localization sequences (NLSs), are set forth in SEQ ID NOs: 25-31. Another exemplary codon optimized nucleic acid sequence encoding a Cas9 molecule of S. aureus comprises the nucleotides 1293-4451 of SEQ ID NO: 32.

b. Second Domain: Modulator of Chromatin Remodeling

The second polypeptide domain comprises a modulator of chromatin structure. The modulator of chromatin structure may also be referred to as a chromatin remodeling protein. The modulator of chromatin structure may have an activity selected from destabilizing histone-DNA interactions, destabilizing nucleosomes, promoting movement of DNA relative to histories (for example, sliding DNA along histones and/or translocating DNA along histones and/or changing the position of a nucleosome, relative to an associated DNA strand), ejecting nucleosomes from a region of DNA, or ejecting histones from the nucleosome, or a combination thereof. In some embodiments, the modulator of chromatin structure comprises a nucleosome rearranging protein. In some embodiments, the modulator of chromatin structure creates nucleosome-depleted region(s) in a gene or genome. For example, the modulator of chromatin structure may comprise the SS18 subunit of the BAF chromatin remodeling complex. The BAF chromatin remodeling complex may also be referred to as the SWItch/Sucrose Non-Fermentable (SWI/SNF) chromatin remodeling complex. SWI/SNF is a subfamily of ATP-dependent chromatin remodeling complexes. The remodeling complex may be composed of several proteins that are products of the SWI and SNF genes, such as SWI1, SWI2/SNF2, SWI3, SWI5, and SWI6. The remodeling complex has a DNA-stimulated ATPase activity that can destabilize histone-DNA interactions in reconstituted nucleosomes in an ATP-dependent manner. The SWI/SNF subfamily may provide nucleosome rearrangement, such as ejection and/or sliding. The movement of nucleosomes may provide easier access to the chromatin, allowing gene expression to be activated or repressed. In some embodiments, the modulator of chromatin structure comprises CHD1 or CHD8 or a variant thereof. In some embodiments, the modulator of chromatin structure comprises the BAF chromatin remodeling complex or a functional subunit thereof or a variant thereof. In some embodiments, the modulator of chromatin structure is a protein that recruits the BAF complex or subunits thereof. The modulator of chromatin structure may comprise the SS18 subunit or a variant thereof. SS18 is a member of the human SWI/SNF chromatin remodeling complex and is involved in chromosomal translocation. 5518 may comprise a polypeptide having the amino acid sequence of SEQ ID NO: 37, encoded by the polynucleotide of SEQ ID NO: 38. In some embodiments, SS18 comprises a polypeptide having at least 75%, at least 80%, at least 85%, at least 90%, at least 95%, at least 98%, at least 99%, or 100% sequence identity to SEQ ID NO: 37, or any range between any two of these values. In some embodiments, the modulator of chromatin structure comprises 1, 2, 3, 4, 5, 6, 7, 8, 9, 10, 11, 12, 13, 14, 15, 18, 17, 18, 19, or 20 amino acid substitutions relative to wild-type SS18 protein. In some embodiments, the modulator of chromatin structure comprises 1, 2, 3, 4, 5, 6, 7, 8, 9, 10, 11, 12, 13, 14, 15, 16, 17, 18, 19, or 20 amino acid substitutions relative to SEQ ID NO: 37, In some embodiments, 5518 comprises a polypeptide encoded by a polynucleotide having at least 75%, at least 80%, at least 85%, at least 90%, at least 95%, at least 98%, at least 99%, or 100% sequence identity to SEQ ID NO: 38, or any range between any two of these values. The modulator of chromatin structure may be from a mammal, such as a mouse or a human, or from another species. In some embodiments, the modulator of chromatin structure is from a mammal. In some embodiments, the modulator of chromatin structure is from a mouse. In some embodiments, the modulator of chromatin structure is from humans. dCas9-SS18 may comprise a polypeptide having the amino acid sequence of SEQ ID NO: 88 or 90, encoded by the polynucleotide of SEQ ID NO: 87 or 89, respectively. In some embodiments, dCas9-SS18 comprises a polypeptide having at least 75%, at least 80%, at least 85%, at least 90%, at least 95%, at least 98%, at least 99%, or 100% sequence identity to SEQ ID NO: 88 or 90, or any range between any two of these values. In some embodiments, dCas9-SS18 comprises a polypeptide encoded by a polynucleotide having at least 75%, at least 80%, at least 85%, at least 90%, at least 95%, at least 98%, at least 99%, or 100% sequence identity to SEQ ID NO: 87 or 89, or any range between any two of these values.

In some embodiments, the modulator of chromatin structure does not have an activity selected from acetyltransferase activity, methyltransferase activity, deacetylase activity, or demethylase activity, or a combination thereof. In some embodiments, the modulator of chromatin structure does not have an activity selected from acetyltransferase activity, methyltransferase activity, deacetylase activity, demethylase activity, covalent histone modification activity, binding to or recruitment of a transcription factor such as a transcription activation factor, or a combination thereof.

The fusion protein comprises one or more second polypeptide domain(s). For example, the fusion protein may include one, two, three, four, or five second polypeptide domains. The first polypeptide domain and the second polypeptide domain(s) may be operably linked. The second polypeptide domain may be at the C-terminal end of the first polypeptide domain, or at the N-terminal end of the first polypeptide domain, or a combination thereof. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three or four) second polypeptide domains in tandem. Each second polypeptide domain may be the same or different. In some embodiments, the fusion protein comprises SS18 fused to the N-terminal end of dCas9 protein. In some embodiments, the fusion protein comprises SS18 fused to the C-terminal end of dCas9 protein.

c. Third Domain: Transcriptional Activator Domain

In some embodiments, the fusion protein further includes one or more third polypeptide domain(s). The third polypeptide domain can have transcription activation activity, for example, a transactivation domain. The transcriptional activator domain may have an activity selected from acetyltransferase activity, methyltransferase activity, deacetylase activity, demethylase activity, or a combination thereof. The transcriptional activator domain may have an activity selected from acetyltransferase activity, methyltransferase activity, deacetylase activity, demethylase activity, covalent histone modification activity, binding to or recruitment of a transcription factor such as a transcription activation factor, or a combination thereof. The transcriptional activator domains may include, for example, a VP16 protein, multiple VP16 proteins such as a VP48 domain or VP64 domain, p65 domain of NF kappa B transcription activator activity, activation domain of HSF1, TET1, VPR, VPH, Rta, p300, or p300 core (p300c), or a combination thereof. The third polypeptide domain may be from a mammal, such as a mouse or a human, or from another species. In some embodiments, the third polypeptide domain is from a mammal. In some embodiments, the transcription activator domain is from mouse. In some embodiments, the transcription activator domain is from human.

The fusion protein may include, for example, one, two, three, four, or five third polypeptide domains. The first polypeptide domain and the second polypeptide domain(s) and the third polypeptide domain(s) may be operably linked. The third polypeptide domain may be at the C-terminal end of the first polypeptide domain, or at the N-terminal end of the first polypeptide domain, or a combination thereof. The fusion protein may include one third polypeptide domain. The fusion protein may include two of the third polypeptide domains. For example, the fusion protein may include a third polypeptide domain at the N-terminal end of the first polypeptide domain as well as a third polypeptide domain at the C-terminal end of the first polypeptide domain. In other embodiments, the fusion protein may include a single first polypeptide domain and more than one (for example, two or three or four) third polypeptide domains in tandem. Each third polypeptide domain may be the same or different.

In some embodiments, the transcriptional activator domain comprises p300. p300 may comprise a polypeptide having the amino acid sequence of SEQ ID NO: 33, or of SEQ ID NO: 34 (p300c). In some embodiments, p300 comprises a polypeptide having at least 75%, at least 80%, at least 85%, at least 90%, at least 95%, at least 98%, at least 99%, or 100% sequence identity to SEQ ID NO: 33 or SEQ ID NO: 34, or any range between any two of these values. dCas9-p300c may comprise a polypeptide having the amino acid sequence of SEQ ID NO: 80 or 82, encoded by the polynucleotide of SEQ ID NO: 79 or 81, respectively. In some embodiments, dCas9-p300c comprises a polypeptide having at least 75%, at least 80%, at least 85%, at least 90%, at least 95%, at least 98%, at least 99%, or 100% sequence identity to SEQ ID NO: 80 or 82, or any range between any two of these values. In some embodiments, dCas9-p300c comprises a polypeptide encoded by a polynucleotide having at least 75%, at least 80%, at least 85%, at least 90%, at least 95%, at least 98%, at least 99%, or 100% sequence identity to SEQ ID NO: 79 or 81, or any range between any two of these values. P300c-dCas9 may comprise a polypeptide having the amino acid sequence of SEQ ID NO: 84 or 86, encoded by the polynucleotide of SEQ ID NO: 83 or 85, respectively. In some embodiments, p300c-dCas9 comprises a polypeptide having at least 75%, at least 80%, at least 85%, at least 90%, at least 95%, at least 98%, at least 99%, or 100% sequence identity to SEQ ID NO: 84 or 86, or any range between any two of these values. In some embodiments, p300c-dCas9 comprises a polypeptide encoded by a polynucleotide having at least 75%, at least 80%, at least 85%, at least 90%, at least 95%, at least 98%, at least 99%, or 100% sequence identity to SEQ ID NO: 83 or 85, or any range between any two of these values. In some embodiments, the fusion protein comprises p300 fused to the N-terminal end of dCas9 protein. In some embodiments, the fusion protein comprises p300 fused to the C-terminal end of dCas9 protein.

In some embodiments, the fusion protein comprises TET1. TET1, also known as Tet1CD (Ten-eleven translocation methylcytosine dioxygenase 1), may comprise a polypeptide having the amino acid sequence of SEQ ID NO: 93, encoded by the polynucleotide of SEQ ID NO: 94. In some embodiments, TET1 comprises a polypeptide having at least 75%, at least 80%, at least 85%, at least 90%, at least 95%, at least 98%, at least 99%, or 100% sequence identity to SEQ ID NO: 93, or any range between any two of these values. In some embodiments, TET1 comprises a polypeptide encoded by a polynucleotide having at least 75%, at least 80%, at least 85%, at least 90%, at least 95%, at least 98%, at least 99%, or 100% sequence identity to SEQ ID NO: 94, or any range between any two of these values. In some embodiments, the fusion protein comprises TET1 fused to the N-terminal end of dCas9 protein. In some embodiments, the fusion protein comprises TET1 fused to the C-terminal end of dCas9 protein.

In some embodiments, the fusion protein comprises VP64. VP64 may comprise a polypeptide having the amino acid sequence of SEQ ID NO: 91, encoded by the polynucleotide of SEQ ID NO: 92. In some embodiments, VP64 comprises a polypeptide having at least 75%, at least 80%, at least 85%, at least 90%, at least 95%, at least 98%, at least 99%, or 100% sequence identity to SEQ ID NO: 91, or any range between any two of these values. In some embodiments, VP64 comprises a polypeptide encoded by a polynucleotide having at least 75%, at least 80%, at least 85%, at least 90%, at least 95%, at least 98%, at least 99%, or 100% sequence identity to SEQ ID NO: 92, or any range between any two of these values. In some embodiments, the fusion protein comprises VP64-dCas9-VP64. VP64-dCas9-VP64 may comprise a polypeptide having the amino acid sequence of SEQ ID NO: 35, encoded by the polynucleotide of SEQ ID NO: 36. In some embodiments, VP64-dCas9-VP64 comprises a polypeptide having at least 75%, at least 80%, at least 85%, at least 90%, at least 95%, at least 98%, at least 99%, or 100% sequence identity to SEQ ID NO: 35, or any range between any two of these values. In some embodiments, VP64-dCas9-VP64 comprises a polypeptide encoded by a polynucleotide having at least 75%, at least 80%, at least 85%, at least 90%, at least 95%, at least 98%, at least 99%, or 100% sequence identity to SEQ ID NO: 36, or any range between any two of these values. In some embodiments, the fusion protein comprises VP64 fused to the N-terminal end of dCas9 protein. In some embodiments, the fusion protein comprises VP64 fused to the C-terminal end of dCas9 protein.

In some embodiments, the transcriptional activator domain comprises VPH, which is a polypeptide comprising VP64, mouse p65, and HSF1. VPH may comprise a polypeptide having the amino acid sequence of SEQ ID NO: 39, encoded by the polynucleotide of SEQ ID NO: 40. In some embodiments, VPH comprises a polypeptide having at least 75%, at least 80%, at least 85%, at least 90%, at least 95%, at least 98%, at least 99%, or 100% sequence identity to SEQ ID NO: 39, or any range between any two of these values. In some embodiments, VPH comprises a polypeptide encoded by a polynucleotide having at least 75%, at least 80%, at least 85%, at least 90%, at least 95%, at least 98%, at least 99%, or 100% sequence identity to SEQ ID NO: 40, or any range between any two of these values, dCas9-VPH may comprise a polypeptide having the amino acid sequence of SEQ ID NO: 72 or 74, encoded by the polynucleotide of SEQ ID NO: 71 or 73, respectively. In some embodiments, dCas9-VPH comprises a polypeptide having at least 75%, at least 80%, at least 85%, at least 90%, at least 95%, at least 98%, at least 99%, or 100% sequence identity to SEQ ID NO: 72 or 74, or any range between any two of these values. In some embodiments, dCas9-VPH comprises a polypeptide encoded by a polynucleotide having at least 75%, at least 80%, at least 85%, at least 90%, at least 95%, at least 98%, at least 99%, or 100% sequence identity to SEQ ID NO: 71 or 73, or any range between any two of these values. VPH-dCas9 may comprise a polypeptide having the amino acid sequence of SEQ ID NO: 68 or 70, encoded by the polynucleotide of SEQ ID NO: 67 or 69, respectively. In some embodiments, VPH-dCas9 comprises a polypeptide having at least 75%, at least 80%, at least 85%, at least 90%, at least 95%, at least 98%, at least 99%, or 100% sequence identity to SEQ ID NO: 68 or 70, or any range between any two of these values. In some embodiments, VPH-dCas9 comprises a polypeptide encoded by a polynucleotide having at least 75%, at least 80%, at least 85%, at least 90%, at least 95%, at least 98%, at least 99%, or 100% sequence identity to SEQ ID NO: 67 or 69, or any range between any two of these values. VPH-dCas9-VPH may comprise a polypeptide having the amino acid sequence of SEQ ID NO: 76, encoded by the polynucleotide of SEQ ID NO: 75. In some embodiments, VPH-dCas9-VPH comprises a polypeptide having at least 75%, at least 80%, at least 85%, at least 90%, at least 95%, at least 98%, at least 99%, or 100% sequence identity to SEQ ID NO: 76, or any range between any two of these values. In some embodiments, VPH-dCas9-VPH comprises a polypeptide encoded by a polynucleotide having at least 75%, at least 80%, at least 85%, at least 90%, at least 95%, at least 98%, at least 99%, or 100% sequence identity to SEQ ID NO: 75, or any range between any two of these values. In some embodiments, the fusion protein comprises VPH fused to the N-terminal end of dCas9 protein. In some embodiments, the fusion protein comprises VPH fused to the C-terminal end of dCas9 protein.

In some embodiments, the transcriptional activator domain comprises VPR, which is a polypeptide comprising VP64, human p65, and Rta. VPR may comprise a polypeptide having the amino acid sequence of SEQ ID NO: 41, encoded by the polynucleotide of SEQ ID NO: 42. In some embodiments, VPR comprises a polypeptide having at least 75%, at least 80%, at least 85%, at least 90%, at least 95%, at least 98%, at least 99%, or 100% sequence identity to SEQ ID NO: 41, or any range between any two of these values. In some embodiments, VPR comprises a polypeptide encoded by a polynucleotide having at least 75%, at least 80%, at least 85%, at least 90%, at least 95%, at least 98%, at least 99%, or 100% sequence identity to SEQ ID NO: 42, or any range between any two of these values. dCas9-VPR may comprise a polypeptide having the amino acid sequence of SEQ ID NO: 78, encoded by the polynucleotide of SEQ ID NO: 77. In some embodiments, dCas9-VPR comprises a polypeptide having at least 75%, at least 80%, at least 85%, at least 90%, at least 95%, at least 98%, at least 99%, or 100% sequence identity to SEQ ID NO: 78, or any range between any two of these values. In some embodiments, dCas9-VPR comprises a polypeptide encoded by a polynucleotide having at least 75%, at least 80%, at least 85%, at least 90%, at least 95%, at least 98%, at least 99%, or 100% sequence identity to SEQ ID NO: 77, or any range between any two of these values. In some embodiments, the fusion protein comprises VPR fused to the N-terminal end of dCas9 protein. In some embodiments, the fusion protein comprises VPR fused to the C-terminal end of dCas9 protein.

In some embodiments, the fusion protein comprises dCas9 protein with VPR fused to its C-terminal end and SS18 fused to its N-terminal end. In some embodiments, the fusion protein comprises dCas9 protein with VPR fused to its N-terminal end and SS18 fused to its C-terminal end. For example, the fusion protein may comprise VPR-dCas9-SS18 or SS18-dCas9-VPR (N-terminal end to C-terminal end) or variants thereof. In some embodiments, the fusion protein comprises dCas9 protein with p300 or p300-core fused to its C-terminal end and SS18 fused to its N-terminal end. In some embodiments, the fusion protein comprises dCas9 protein with p300 or p300-core fused to its N-terminal end and SS18 fused to its C-terminal end. For example, the fusion protein may comprise p300-dCas9-SS18 or SS18-dCas9-p300 (N-terminal end to C-terminal end) or variants thereof. In some embodiments, the fusion protein comprises dCas9 protein with VP64 fused to its C-terminal end and SS18 fused to its N-terminal end. In some embodiments, the fusion protein comprises dCas9 protein with VP64 fused to its N-terminal end and SS18 fused to its C-terminal end. For example, the fusion protein may comprise VP64-dCas9-SS18 or SS18-dCas9-VP64 (N-terminal end to C-terminal end) or variants thereof. In some embodiments, the fusion protein comprises dCas9 protein with VPH fused to its C-terminal end and SS18 fused to its N-terminal end. In some embodiments, the fusion protein comprises dCas9 protein with VPH fused to its N-terminal end and SS18 fused to its C-terminal end. For example, the fusion protein may comprise VPH-dCas9-SS18 or SS18-dCas9-VPH (N-terminal end to C-terminal end) or variants thereof. VPH-dCas9-SS18 may comprise a polypeptide having the amino acid sequence of SEQ ID NO: 64 or 66, encoded by the polynucleotide of SEQ ID NO: 63 or 65, respectively. In some embodiments, VPH-dCas9-SS18 comprises a polypeptide having at least 75%, at least 80%, at least 85%, at least 90%, at least 95%, at least 98%, at least 99%, or 100% sequence identity to SEQ ID NO: 64 or 66, or any range between any two of these values. In some embodiments, VPH-dCas9-SS18 comprises a polypeptide encoded by a polynucleotide having at least 75%, at least 80%, at least 85%, at least 90%, at least 95%, at least 98%, at least 99%, or 100% sequence identity to SEQ ID NO: 63 or 65, or any range between any two of these values.

### 3. DNA Targeting System

Provided herein are DNA Targeting Systems. The DNA Targeting System may be used to activate transcription or increase expression of a gene. The DNA Targeting System includes at least one fusion protein as detailed herein. In embodiments wherein the DNA binding protein of the fusion protein comprises a Cas protein such as Cas9, the DNA Targeting System may further include at least one gRNA.

a. Guide RNA (gRNA)

The at least one gRNA molecule can bind and recognize a target region. The gRNA provides the targeting of a Cas9 DNA targeting system, which may also be referred to as a CRISPR/Cas9-based gene editing system. The gRNA is a fusion of two noncoding RNAs: a crRNA and a tracrRNA, gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 by protospacer which confers targeting specificity through complementary base pairing with the desired DNA target. The “target region” or “target sequence” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to the sequence that the gRNA targets. Together, the gRNA targeting portion and gRNA scaffold form one polynucleotide. The constant region of the gRNA may include the sequence of SEQ ID NO: 62 (RNA), which is encoded by a sequence comprising SEQ ID NO: 61 (DNA). The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

The gRNA may target an exon of a gene. The gRNA may target a region of a gene that is non-coding, such as a regulatory region or promoter sequence. The gRNA may target an intron of a gene. In some embodiments, the gRNA corresponds to a polynucleotide sequence selected from SEQ ID NOs: 43-54, a complement of, a truncation thereof, or a variant thereof (TABLE 1). The gRNA may be encoded by or target or bind to or hybridize to a target sequence selected from SEQ ID NOs: 43-48, a complement thereof, a truncation thereof, or a variant thereof. The gRNA may comprise a polynucleotide sequence selected from SEQ ID NOs: 49-54, a complement thereof, a truncation thereof, or a variant thereof. A truncation may be, for example, 1, 2, 3, 4, 5, 8, 7, 8, or 9 nucleotides shorter than the reference sequence. The DNA Targeting System may include one or more gRNAs, each gRNA corresponding to a polynucleotide sequence selected from SEQ ID NOs: 43-54, a complement thereof, a truncation thereof, or a variant thereof, or each gRNA corresponding to a polynucleotide sequence having at least 70%, at least 75%, at least 80%, at least 85%, at least 90%, or at least 95% sequence identity to at least one of SEQ ID NOs: 43-54, a complement thereof, a truncation thereof, or a variant thereof.

TABLE 1
Examples of gRNAs used to activate
expression of HBG1/2.

| Name | gRNA target/ | gRNA |
|---|---|---|
| | sequence | |
| gRNA1 | TAGTCTTAGA | UAGUCUUAGAGU |
| promoter | GTATCCAGTG | AUCCAGUG |
| HBG1/2 | (SEQ ID NO: 43) | (SEQ ID NO: 49) |
| gRNA2 | GGCTAGGGATG | GGCUAGGGAU |
| promoter | AAGAATAAA | GAAGAAUAAA |
| HBG1/2 | (SEQ ID NO: 44) | (SEQ ID NO: 50) |
| gRNAI HS2 | AATATGTCACA | AAUAUGUCAC |
| enhancer | TTCTGTCTC | AUUCUGUCUC |
| HBG1/2 | (SEQ ID NO: 45) | (SEQ ID NO: 51) |
| gRNA2HS2 | GGACTATGGG | GGACUAUGGG |
| enhancer | AGGTCACTAA | AGGUCACUAA |
| HBG1/2 | (SEQ ID NO: 46) | (SEQ ID NO: 52) |
| gRNA3HS2 | GAAGGTTACAC | GAAGGUUACA |
| enhancer | AGAACCAGA | CAGAACCAGA |
| HBG1/2 | (SEQ ID NO: 47) | (SEQ ID NO: 53) |
| gRNA4 HS2 | GCCCTGTAAGC | GCCCUGUAAGC |
| enhancer | ATCCTGCTG | AUCCUGCUG |
| HBG1/2 | (SEQ ID NO: 48) | (SEQ ID NO: 54) |

### 4. Genetic Constructs

The fusion protein or DNA Targeting system or a component thereof may be encoded by or comprised within a genetic construct. The DNA Targeting system may comprise one or more genetic constructs. The genetic construct, such as a plasmid or expression vector, may comprise a nucleic acid that encodes the fusion protein or DNA Targeting system and/or at least one of the gRNAs. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, a first gRNA molecule, and optionally a Cas9 molecule or fusion protein, and a second genetic construct encodes one gRNA molecule, i.e., a second gRNA molecule, and optionally a Cas9 molecule or fusion protein.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the fusion protein or DNA Targeting system and may further comprise an initiation codon, which may be upstream of the fusion protein or DNA Targeting system coding sequence, and a stop codon, which may be downstream of the fusion protein or DNA Targeting system coding sequence. The initiation and termination codon may be in frame with the fusion protein or DNA Targeting system coding sequence. The vector may also comprise a promoter that is operably linked to the fusion protein or DNA Targeting system coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The fusion protein or DNA Targeting system may be under the light-inducible or chemically inducible control to enable the dynamic control of gene/genome editing in space and time. The promoter operably linked to the fusion protein or DNA Targeting system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Patent Application Publication No. 0520040175727, the contents of which are incorporated herein in its entirety. The promoter may be a CK8 promoter, a Spc512 promoter, a MHCK7 promoter, for example.

The genetic construct may also comprise a polyadenylation signal, which may be downstream of the fusion protein or DNA Targeting system. The polyadenylation signal may be a SV40 polyadenylation signal, LTR polyadenylation signal, bovine growth hormone (bGH) polyadenylation signal, human growth hormone (hGH) polyadenylation signal, or human p-globin polyadenylation signal. The SV40 polyadenylation signal may be a polyadenylation signal from a pCEP4 vector (Invitrogen, San Diego, Calif.).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the fusion protein or DNA Targeting system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting cells with nucleic acid encoding the fusion protein or DNA Targeting system, which the transformed host cell is cultured and maintained under conditions wherein expression of the fusion protein or DNA Targeting system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver fusion proteins or DNA Targeting systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have tissue-specific tropism. The modified AAV vector may be capable of delivering and expressing the fusion protein or DNA Targeting system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative tissue-tropic AAV capsids, such as AAV2/1, AAV2/6, AAV2/7, AAV2/8, AAV2/9, AAV2.5, and AAV/SASTG vectors that efficiently transduce target tissue by systemic and local delivery (Seto et al. Current Gene Therapy 2012, 12, 139-151). The modified AAV vector may be AAV2i8G9 (Shen et al. J. Biol. Churn. 2013, 288, 28814-28823).

### 5. Pharmaceutical Compositions

Further provided herein are pharmaceutical compositions comprising the above-described fusion proteins or DNA Targeting systems or genetic constructs. In some embodiments, the pharmaceutical composition may comprise about 1 ng to about 10 mg of DNA encoding the fusion protein or DNA Targeting system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 6. Administration

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibias anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of fusion proteins or DNA Targeting systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a DNA targeting system as detailed herein. Suitable cell types are detailed herein. In some embodiments, the cell is an immune cell. Immune cells may include, for example, lymphocytes such as T cells and B cells and natural killer (NK) cells. In some embodiments, the cell is a T cell. T cells may be divided into cytotoxic T cells and helper T cells, which are in turn categorized as TH1 or TH2 helper T cells. Immune cells may further include innate immune cells, adaptive immune cells, tumor-primed T cells, NIST cells, IFN-γ producing killer dendritic cells (IKDC), memory T cells (TCMs), and effector T cells (TEs). The cell may be a stem cell such as a human stem cell. In some embodiments, the cell is an embryonic stem cell or a hematopoietic stem cell. The stem cell may be a human induced pluripotent stem cell (iPSCs). Further provided are stem cell-derived neurons, such as neurons derived from iPSCs transformed or transduced with a DNA targeting system or component thereof as detailed herein. The cell may be a muscle cell. Cells may further include, but are not limited to, immortalized myoblast cells, dermal fibroblasts, primal dermal fibroblasts, bone marrow-derived progenitors, skeletal muscle progenitors, human skeletal myoblasts, CD 133+ cells, mesoangioblasts, cardiomyocytes, hepatocytes, chondrocytes, mesenchymal progenitor cells, hematopoietic stem cells, smooth muscle cells, dendritic cells, and MyoD- or Pax7-transduced cells, or other myogenic progenitor cells.

### 7. Kits

Provided herein is a kit, which may be used to enhance or increase expression of a gene. The kit comprises genetic constructs or a composition comprising the same, as described above, and instructions for using said composition. In some embodiments, the kit comprises at least one fusion protein, and instructions for using the fusion protein.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an Internet site that provides the instructions.

### 8. Methods

a. Methods of Activating Expression of a Target Gene

Provided herein are methods of activating expression of a target gene in a cell. The methods may include contacting the cell with a fusion protein as detailed herein or a DNA Targeting System as detailed herein or a gRNA as detailed herein. In some embodiments, the target gene is a gamma globin gene. In some embodiments, the gene is gamma globin genes 1 and 2 (HBG1/2). In some embodiments, the DNA Targeting System includes at least one gRNA corresponding to at least one of SEQ ID NOs: 43-54 as detailed herein. The DNA Targeting system may target, for example, the promoter of HBG1/2 and/or the HS2 enhancer region of HBG1/2. In some embodiments, one or two gRNAs are used to target the promoter of HBG1/2 to activate its expression. For example, the DNA Targeting System may include one or two gRNAs that are encoded by or target a sequence of SEQ ID NO: 43 and/or 44, to target the promoter of HBG1/2 to activate its expression. The DNA Targeting System may include one or two gRNAs comprising a polynucleotide sequence selected from SEQ ID NOs: 49 and 50, to target the promoter of HBG1/2 to activate its expression. In some embodiments, four gRNAs are used to target the HS2 enhancer region of HBG1/2 to activate its expression. For example, the DNA Targeting System may include one, two, three, or four gRNAs that are encoded by or target a sequence selected from SEQ ID NOs: 45-48, to target the HS2 enhancer region of HBG1/2 to activate its expression. The DNA Targeting System may include one, two, three, or four gRNAs comprising a polynucleotide sequence selected from SEQ ID NOs: 51-54, to target the HS2 enhancer region of HBG1/2 to activate its expression. In some embodiments, methods disclosed herein increase mRNA expression of the target gene in a cell compared to a control. The control may be the mRNA expression of the target gene in a cell in which the fusion protein is not present. In some embodiments, methods disclosed herein increase the level of protein expressed from the target gene in a cell compared to a control. The control may be the level of protein expression from the target gene in a cell in which the fusion protein is not present.

### 9. Examples

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Combining Chromatin Remodelers with Activator Domains Enhances Activation of Target Gene Expression

Targeted activation of endogenous genes with synthetic transcription factors or epigenome editors, made from DNA-targeting systems such as zinc finger proteins, TALEs, and CRISPR-Cas systems, are broadly useful for gene therapy, regenerative medicine, and programming stem cell differentiation. However, a common limitation is that the potency of gene activation is insufficient to generate the desired phenotype or biological effect. Here, it is demonstrated that combining modulators of chromatin structure (for example, SS18 or the SWI/SNF (BAF) chromatin remodeling complex) with activator domains (for example, VP64, VPH, VPR, and/or p300) can lead to more potent gene activation in human cells.

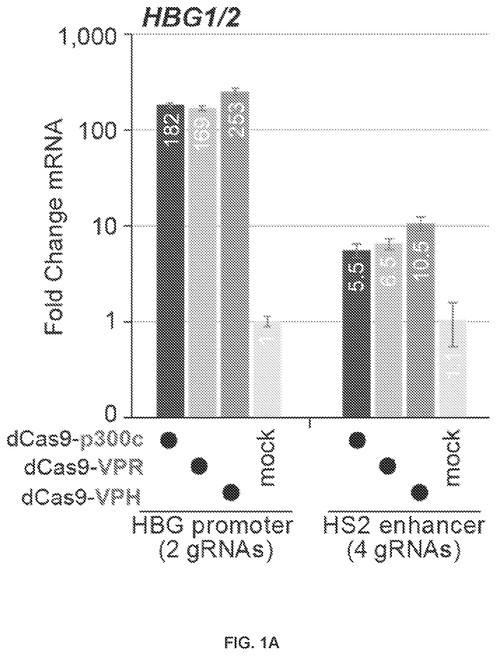

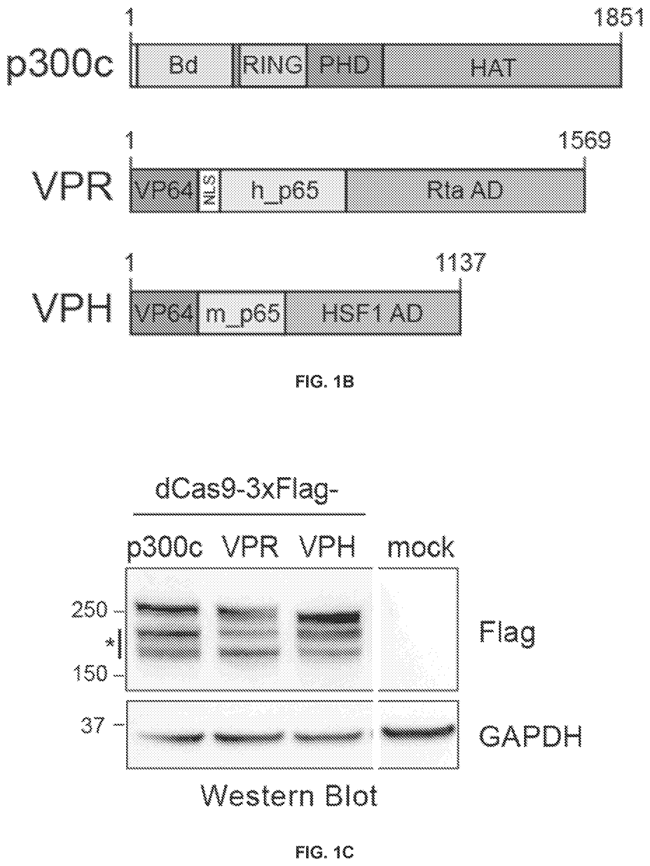

In an effort to generate more potent transcriptional activators relative to state-of-the-art by rational design of programmable gene modulators, the potency of dCas9-VPH to activate the HBG1/2 gene relative to p300core (p300c) and VPR fusions was compared (FIG. 1A). VPH, a fusion of VP64, mouse p65 activation domain (AD), and HSF1 (AD), has been used in few instances but is less commonly used than VPR (VP64, human p65 activation domain (AD), and Rta) (FIG. 18). All three effectors lead to deposition of histone H3K27 acetylation, a mark of gene activation, as well as recruitment of transcription factors. dCas9-VPH was more potent than p300c or VPR in activating HBG1/2 when targeted to either its promoter or enhancer (FIG. 1C).

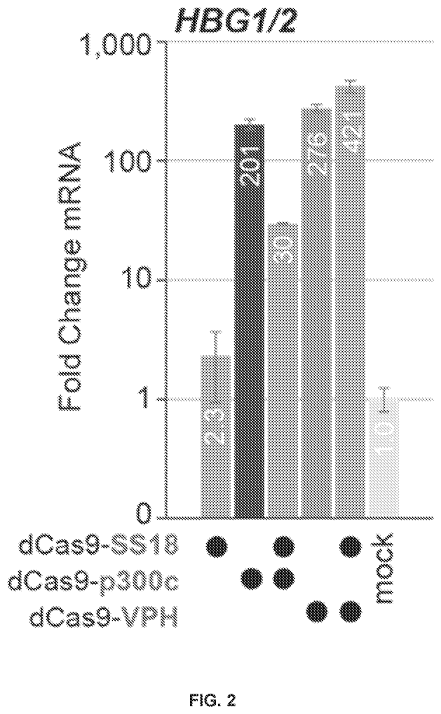

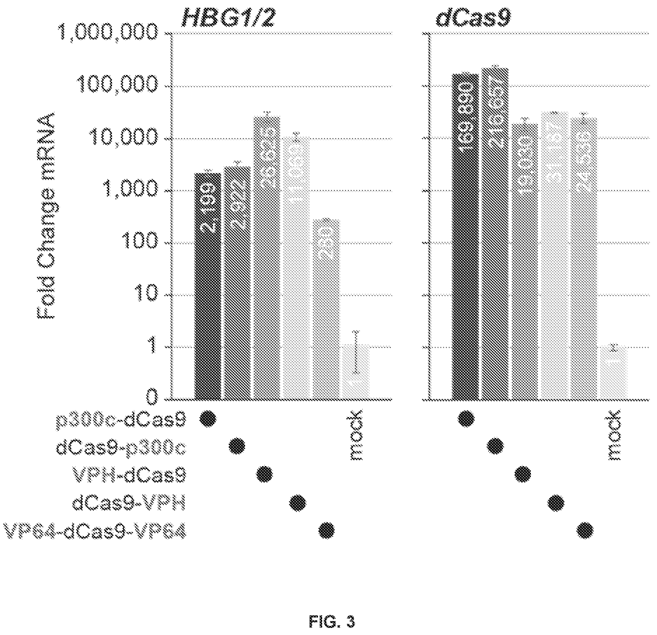

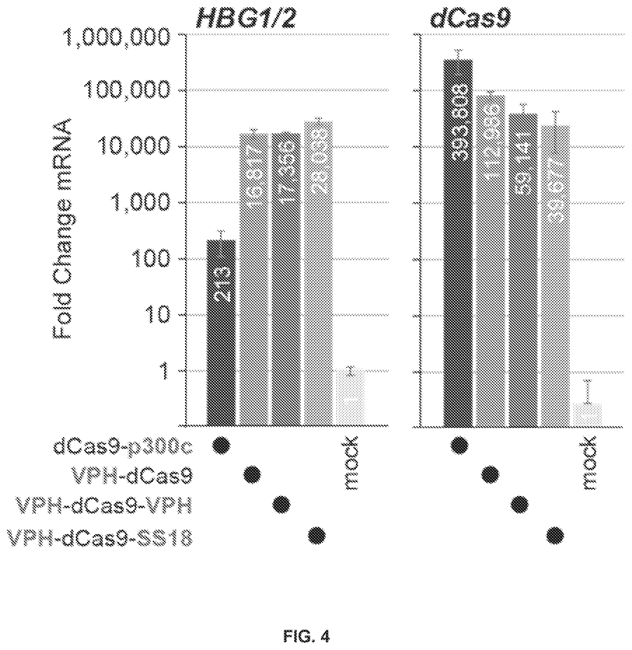

The SWI/SNF (BAF) chromatin remodeling complex has been shown to antagonize PRC1/2 complexes that deposit and bind to the repressive H3K27 trimethylation and H2A ubiquitylation histone marks, leading to a more facultative chromatin state. To design a more potent dCas9 activator, the potency of dCas9-VPH or dCas9-p300c activating HBG1/2 when used in combination with a dCas9-SS18 fusion was examined, since the SS18 subunit of the BAF complex is sufficient to recruit the full BAF complex to chromatin. Using dCas9-SS18 in combination with dCas9-VPH showed greater activation of HBG1/2 compared with that achieved by its combination with dCas9-p300c or any of the dCas9-fusions alone (FIG. 2). Fusion of several transcriptional regulators to dCas9 in tandem can lead to a synergistic increase in activity. To generate a dCas9 bipartite activator consisting of VPH and SS18, it was examined whether VPH was a more potent activator when fused to dCas9 at its N-terminus or C-terminus. dCas9 fused to p300c or to two VP64 domains (one on each termini) were also included as controls. VPH showed the strongest activation of HBG1/2 when fused at the N-terminus of dCas9 and was the most potent of all the activators tested (FIG. 3). For the design of the bipartite dCas9 activator, VPH was fused on the N-terminus of dCas9 and SS18 on its C-terminus. When tested for HBG1/2 activation in a side-by-side comparison of the best dCas9-fusion activators, the VPH-dCas9-SS18 bipartite activator outperformed both p300c and VPH as a single fusion to dCas9 (FIG. 4).

Collectively, these results support a model in which remodelers of chromatin structure cooperate with co-recruited transcriptional activation domains to more robustly activate target gene expression.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A fusion protein comprising at least two heterologous polypeptide domains, wherein the first polypeptide domain comprises a DNA binding protein and the second polypeptide domain comprises a modulator of chromatin structure.

Clause 2. The fusion protein of clause 1, wherein the fusion protein further comprises a third polypeptide domain.

Clause 3. The fusion protein of any one of the preceding clauses, wherein the first polypeptide domain comprises a CRISPR-associated (Gas) protein, a TALE, or a zinc finger protein.

Clause 4. The fusion protein of clause 3, wherein the Cas protein comprises at least one amino acid mutation that eliminates nuclease activity of the Cas protein.

Clause 5. The fusion protein of clause 3 or 4, wherein the Cas protein comprises a Cas9 protein.

Clause 6. The fusion protein of clause 5, wherein the Cas9 protein is nuclease-deficient dCas9 and comprises a polypeptide having at least 75% sequence identity to SEQ ID NO: 20 or 21 or is encoded by a polynucleotide comprising a sequence having at least 75% identity to SEQ ID NO: 22 or 23.

Clause 7. The fusion protein of any one of the preceding clauses, wherein the modulator of chromatin structure comprises a nucleosome rearranging protein.

Clause 8. The fusion protein of any one of the preceding clauses, wherein the modulator of chromatin structure comprises the SS18 subunit of the BAF chromatin remodeling complex or a fragment thereof or a variant thereof.

Clause 9. The fusion protein of clause 8, wherein the SS18 subunit comprises a polypeptide having at least 75% sequence identity to SEQ ID NO: 37.

Clause 10. The fusion protein of any one of clauses 2-9, wherein the third polypeptide domain comprises a transcriptional activator domain.

Clause 11. The fusion protein of clause 10, wherein the transcriptional activator domain comprises VP64, VPH, VPR, p65, TET1, or p300, or a combination thereof or a fragment thereof or a variant thereof.

Clause 12. The fusion protein of clause 11, wherein the VP64 comprises a polypeptide having at least 75% sequence identity to SEQ ID NO: 91.

Clause 13. The fusion protein of clause 11, wherein the TET1 comprises a polypeptide having at least 75% sequence identity to SEQ ID NO: 93.

Clause 14. The fusion protein of clause 11, wherein the VPH comprises a polypeptide having at least 75% sequence identity to SEQ ID NO: 39.

Clause 15. The fusion protein of clause 11, wherein the VPR comprises a polypeptide having at least 75% sequence identity to SEQ ID NO: 41.

Clause 16. The fusion protein of clause 11, wherein the p300 comprises a polypeptide having at least 75% sequence identity to SEQ ID NO: 33 or 34.

Clause 17. The fusion protein of any one of clauses 1-16, wherein the fusion protein comprises one or more second polypeptide domain(s).

Clause 18. The fusion protein of clause 17, wherein the one or more second polypeptide domain(s) is fused to the C-terminus or the N-terminus of the first polypeptide domain, or a combination thereof.

Clause 19. The fusion protein of clause 18, wherein the N-terminus of the second polypeptide is operably linked to the C-terminus of the first polypeptide domain, or wherein the C-terminus of the second polypeptide is operably linked to the N-terminus of the first polypeptide domain.

Clause 20. The fusion protein of any one of clauses 2-19, wherein the fusion protein comprises one or more third polypeptide domain(s).

Clause 21. The fusion protein of clause 20, wherein the one or more third polypeptide domain is fused to the C-terminus or the N-terminus of the first polypeptide domain, or a combination thereof.

Clause 22. The fusion protein of clause 21, wherein the N-terminus of the third polypeptide is operably linked to the C-terminus of the first polypeptide domain, or wherein the C-terminus of the third polypeptide is operably linked to the N-terminus of the first polypeptide domain.

Clause 23. The fusion protein of any one of clauses 2-22, wherein the first polypeptide domain comprises dCas9, wherein the second polypeptide domain comprises SS18, and wherein the third polypeptide domain comprises VPH.

Clause 24. The fusion protein of clause 23, wherein the fusion protein comprises VPH-dCas9-SS18 or SS18-dCas9-VPH or variants thereof.

Clause 25. The fusion protein of clause 24, wherein the fusion protein comprises a polypeptide having at least 75% sequence identity to SEQ ID NO: 64 or 66.

Clause 26. The fusion protein of any one of clauses 2-22, wherein the first polypeptide domain comprises dCas9, wherein the second polypeptide domain comprises 8518, and wherein the third polypeptide domain comprises VPR.

Clause 27. The fusion protein of clause 26, wherein the fusion protein comprises VPR-dCas9-SS18 or 8518-dCas9-VPR or variants thereof.

Clause 28. The fusion protein of any one of clauses 2-22, wherein the first polypeptide domain comprises dCas9, wherein the second polypeptide domain comprises SS18, and wherein the third polypeptide domain comprises p300.

Clause 29. The fusion protein of clause 28, wherein the fusion protein comprises p300-dCas9-SS18 or SS18-dCas9-p300 or variants thereof.

Clause 30. The fusion protein of any one of clauses 2-22, wherein the first polypeptide domain comprises dCas9, wherein the second polypeptide domain comprises SS18, and wherein the third polypeptide domain comprises VP64.

Clause 31. The fusion protein of clause 30, wherein the fusion protein comprises VP64-dCas9-SS18 or SS18-dCas9-VP64 or variants thereof.

Clause 32. The fusion protein of any one of the preceding clauses, wherein the fusion protein activates transcription of a target gene.

Clause 33. The fusion protein of any one of the preceding clauses, wherein the fusion protein increases the level of mRNA expression of a target gene in a cell containing the fusion protein relative to a control.

Clause 34. The fusion protein of clause 33, wherein the level of mRNA expression of the target gene is increased at least 5-fold, at least 50-fold, at least 100-fold, at least 1,000-fold, at least 10,000-fold, or at least 20,000-fold relative to a control.

Clause 35. The fusion protein of clause 33 or 34, wherein the level of mRNA expression of the target gene is increased by 5-fold to 10,000-fold, 5-fold to 30,000-fold, 5-fold to 50,000-fold, 5-fold to 100,000-fold, 10,000-fold to 30,000-fold, 20,000-fold to 30,000-fold, 15,000-fold to 25,000-fold, 1,000-fold to 50,000-fold, or 1,000-fold to 100,000-fold relative to a control.

Clause 36. The fusion protein of any one of clauses 33-35, wherein the control is the level of mRNA expression of the target gene in a cell not containing the fusion protein.

Clause 37. The fusion protein of any one of clauses 32-36, wherein the target gene is gamma globin genes 1 and 2 (HBG1/2).

Clause 38. A DNA Targeting System comprising: (a) the fusion protein of any one of clauses 1-37, wherein the first polypeptide domain comprises a zinc finger protein or a TALE; or (b) a gRNA and the fusion protein of any one of clauses 1-37, wherein the first polypeptide domain comprises a Cas protein, and wherein the gRNA targets a target gene.

Clause 39. The DNA Targeting System of clause 38, wherein gRNA targets a regulatory region of the target gene.

Clause 40. The DNA Targeting System of clause 39, wherein the regulatory region is a promoter sequence of the target gene.

Clause 41. A DNA Targeting System comprising a gRNA that recruits a modulator of chromatin structure to a target sequence.

Clause 42. The DNA Targeting System of clause 41, wherein the modulator of chromatin structure comprises the SS18 subunit of the BAF chromatin remodeling complex.

Clause 43. The DNA Targeting System of any one of clauses 38-42, wherein the gRNA is encoded by or binds to a target sequence selected from SEQ ID NOs: 43-48, a complement thereof, a truncation thereof, or a variant thereof, or wherein the gRNA is encoded by or binds to a target sequence having at least 70% sequence identity to a sequence selected from SEQ ID NOs: 43-48, a complement thereof, a truncation thereof, or a variant thereof.

Clause 44. The DNA Targeting System of any one of clauses 38-43, wherein the gRNA comprises a polynucleotide sequence selected from SEQ ID NOs: 49-54, a complement thereof, a truncation thereof, or a variant thereof, or wherein the gRNA comprises a polynucleotide having at least 70% sequence identity to a sequence selected from SEQ ID NOs: 49-54, a complement thereof, a truncation thereof, or a variant thereof.

Clause 45. A method of increasing expression of a target gene in a cell, the method comprising contacting the cell with the fusion protein of any one of clauses 1-37 or the DNA Targeting system of any one of clauses 38-44.

Clause 46. The method of clause 45, wherein the target gene is gamma globin genes 1 and 2 (HBG1/2).

Clause 47. A gRNA encoded by or binding to a target sequence selected from SEQ ID NOs: 43-48, a complement thereof, a truncation thereof, or a variant thereof, or comprising a polynucleotide sequence selected from SEQ ID NOs: 49-54, a complement thereof, a truncation thereof, or a variant thereof.

SEQUENCES
SEQ ID NO: 1
NRG
(R = A or G; N can be any nucleotide residue, e.g., any of A, G, C, or T)
SEQ ID NO: 2
NGG
(N can be any nucleotide residue, e.g., any of A, G, C, or T)
SEQ ID NO: 3
NAG
(N can be any nucleotide residue, e.g., any of A, G, C, or T)
SEQ ID NO: 4
NGGNG
(N can be any nucleotide residue, e.g., any of A, G, C, or T)
SEQ ID NO: 5
NNAGAAW
(W = A or T; N can be any nucleotide residue, e.g,, any of A, G, C, or T)
SEQ ID NO: 6
NAAR
(R = A or G; N can be any nucleotide residue, e.g., any of A, G, C, or T)
SEQ ID NO: 7
NNGRR
(R = A or G; N can be any nucleotide residue, e.g., any of A, G, C, or T)
SEQ ID NO: 8
NNGRRN
(R = A or G; N can be any nucleotide residue, e.g., any of A, G, C, or T)
SEQ ID NO: 9
NNGRRT
(R = A or G; N can be any nucleotide residue, e.g., any of A, G, C, or T)
SEQ ID NO: 10
NNGRRV
(R = A or G; N can be any nucleotide residue, e.g., any of A, G, C, or T)
SEQ ID NO: 11
NNNNGATT
(N can be any nucleotide residue, e.g., any of A, G, C, or T)
SEQ ID NO: 12
NNNNGNNN
(N can be any nucleotide residue, e.g., any of A, G, C, or T)
SEQ ID NO: 13
NGA
(N can be any nucleotide residue, e.g., any of A, G, C, or T)
SEQ ID NO: 14
NNNRRT
(R = A or G; N can be any nucleotide residue, e.g., any of A, G, C, or T)
SEQ ID NO: 15
ATTCCT
SEQ ID NO: 16
NGAN
(N can be any nucleotide residue, e.g., any of A, G, C, or T)
SEQ ID NO: 17
NGNG
(N can be any nucleotide residue, e.g., any of A, G, C, or T)
Streptococcus pyogenes Cas9
SEQ ID NO: 18
MDKKYSIGLDIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
Staphylococcus aureus Cas9 molecule
SEQ ID NO: 19
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKE
IIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
Streptococcus pyogenes Cas9
(with D10A)
SEQ ID NO: 20
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKKLPNEKVLPKHSLLYEYFTVYMELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
Streptococcus pyogenes Cas9
(with D10A, H849A)
SEQ ID NO: 21
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNEMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIBTNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
Polynucleotide sequence of D10A mutant of S. aureus Cas9
SEQ ID NO: 22
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aactaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
Polynucleotide sequence of N580A mutant of S. aureus Cas9
SEQ ID NO: 23
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctetggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaageag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatcata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
codon optimized polynucleotide encoding S. pyogenes Cas9
SEQ ID NO: 24
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cactctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atccaccgga aacggtatac cagtaccaaa
gaggtcctcg atgccaccct catccaccag tcaattactg gcctgtacga aacacggatc
gacctctctc aactgggcgg cgactag
codon optimized nucleic acid sequences encoding S. aureus Cas9
SEQ ID NO: 25
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagacca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccacgaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgcgtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcac.cc tcagattatc aaaaagggc
codon optimized nucleic acid sequences encoding S. aureus Cas9
SEQ ID NO: 26
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
ccagagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa atggaagttt aagaaagagc ggaacaaggg gtacaagcac
cacgccgagg acgccctgat cattgccaac gccgatttca tcttcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
codon optimized nucleic acid sequence encoding S. aureus Cas9
SEQ ID NO: 27
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgc gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgacca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcatc aagaaggga
codon optimized nucleic acid sequence encoding S. aureus Cas9
SEQ ID NO: 28
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagactggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
codon optimized nucleic acid sequence encoding S. aureus Cas9
SEQ ID NO: 29
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgaggcacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggc acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agaccaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
ttc
codon optimized nucleic acid sequences encoding S. aureus Cas9
SEQ ID NO: 30
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagcrggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaaattccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccctcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
coccgaaaagctgctgacgtaccaccacgacccccagacctacoagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
codon optimized nucleic acid sequences encoding S. aureus Cas9
SEQ ID NO: 31
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtaatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
Vect or (pD0242) encoding codon optimized nucleic acid sequence
encoding S. aureus Cas9
SEQ ID NO: 32
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtetatataagcagagctctctggctaactaccggtgccacc
ATGAAAAGGAACTACATTCTGGGGCTGGACATCGGGATTACAAGCGTGGCTTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCGAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAAGAGCAAGGACGCACAGAAGATGATCAATGAGATGCAGAAACGAAACCG
GCAGACGAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGOTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctccccegtgcct
tccttgaccctggaaggtgccactcccactgtcctttcctaataaaatgaggaaattgcatcgcattg
tctgagtaggtctcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcctttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtotcatgagc
ggatacatatttgaatgtatttagaaaaataaacaaataggggttccgcgcacatttccccgaaaagt
gccac
Human p300
(with L553M mutation) protein
SEQ ID NO: 33
MAENVVEPGPPSAKRPKLSSPALSASASDGTDFGSLFDLEHDLPDELINSTELGLTNGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMN8PVNQPAMGMNTGMNAGMNPGMLAAGNGQGIMPNQVMNGSIGAGRGRQK
MQYPNPGMGSAGNLLTEPLQQGSPQMGGQTGLRGPQPLKMGMMNNPNPYGSPYTQNPGQQIGASGLGL
QTQTKTVLSNNLSPFAMDKKAVPGGGMPNMGQQPAPQVQQPGLVTPVAQGMGSGAHTADPEKRKLIQQ
QLVLLLHAHKCQRREQANGEVRQCNLPHCRTMKNVLNHMTHCQSGKSCQVAHCASSRQIISHWKNCTR
KDCPVCLPLKNAGDKRNQQPILTGAPVGLGNPSSLGVGQQSAPKLSTVSQIDPSSIERAYAALGLPYQ
VNQMPTQPQVOAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNQGQFLPQTQFPSQGMNVTNIPLAPSSGQAPVSGAQMSSSSCPVNSPIMPPGSQGSH
IHCPQLPQPALHQNSPSPVPSRTPTPHHTPPSIGAQQPPATTIPAPVPTPPAMPPGPQSOALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPSSLPFRQPVDPQLLGIPD
YEDIVKSPMDLSTIKRKLDTGQYQEPWQYVDDIWLMFNNAWLYNRKTSRVYKYCSKLSEVFEQEIDPV
MQSLGYCCGRKLEFSPQTLCCYGKQLCTIPRDATYYSYQKRYHFCEKCFNEIQGESVSLGDDPSQPQT
TINKEQFSKRKNDTLDPELFVECTECGRKMHQICVLHHEIIWPAGFVCDGCLKKSARTRKENKFSAKR
LPSTRLGTFLENRVNDFLRRQNKPESGEVTVRVVHASDKTVEVKPGMKARFVDSGEMAESFPYRTKAL
FAFEEIDGVDLCFFGMHVQEYGSDCPPPNQRRVYISYLDSVHFFRPKCLRTAVYHEILIGYLEYVKKL
GYTTGHIWACPPSEGDDYIFHCHPPDQKIPKPKRLQEWYKKMLDKAVSERIVHDYKDIFKQATEDRLT
SAKELPYFEGDFWPNVLEESIKELEOEEEERKREENTSNESTDVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMLVELHTQSQDRFVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKTNGGCPICKQLIALCCYHAKHCQENKCPVPFCLNIKQKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFTKQRAAKYANSNPQPIPGQPGMPQ
GQPGLQPPTMPGQQGVHSNPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSQPPHSSPSPRMQPQPSPHHVSPQTSSPHPGLVAAQANPMEQGHFASPDQNSMLSQLASNP
GMANLHGASATDLGLSTDNSDLNSNLSQSTLDIH
Human p300 Core Effect or protein
(aa 1048-1664 of SEQ ID NO: 33)
SEQ ID NO: 34
IFKPEELRQALMPTLEALYRQDPESLPFRQPVDPQLLGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQTLCCYGKQLC
TIPRDATYYSYONRYHFCEKCFNEIOGESVSLGDDPSQPOTTINKEOFSKRKNDTLDPELFVECTECG
RKMHOICVLHHEITWPAGFVCDGCLKKSARTRKENKFSAKRLPSTRLGTFLENRVNDFLRRONHPESG
EVTVRVVHASDKTVEVKPGMKARFVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDFWPNVLEESIKELEQE
EEERKREENTSNESTDVTKGDSKNAKKKNNKKTSKNKSSLSRGNKKKPGMPNVSNDLSQKLYATMEKH
KEVFFVTRLIAGPAANSLPPIVDPDPLIPCDLMDGRDAFLTIARDKHLEFSSLRRAQWSTMCMLVELH
TQSQF
VP64-dCas9-VP64 protein
(with dCas9 underlined)
SEQ ID NO: 35
RADALDDFDLDMLGSDALDDFDLDMLGSDALDDFDLDMLGSDALDDFDLDMVNPKKKRKVGRGMDKKY
SIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRIDLSQL
GGDSRADPKKKRKVASRADALDDFDLDMLGSDALDDFDLDMLGSDALDDFDLDMLGSDALDDFDLDML
I
VP64-dCas9-VP64 DNA
SEQ ID NO: 36
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctga;acctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacttcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcctcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctsgtgcgggcgagctgcagaaaggtaacgsgctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
Protein sequence f or SS18
SEQ ID NO: 37
MSVAFAAPRQRGKGEITPAAIQKMLDDNNHLIQCIMDSQNKGKTSECSQYQQMLHTNLVYLATIADSN
QNMQSLLPAPPTQNMPMGPGGMNQSGPPPPPRSHNMPSDGMVGGGPPAPHMQNQMNGQMPGPNHMPMQ
GPGPNOLNMTNSSMNMPSSSHGSMGGYNHSVPSSOSMPVONOMTMSQGOPMGNYGPRPNMSMOPNOGP
MMHQQPPSQQYNMPQGGGQHYQGQQPPMGMMGQVNQGNHMMGQRQIPPYRPPQQGPPQQYSGQEDYYG
DQYSHGGQGPPEGMNQQYYPDGNSQYGQQQDAYQGPPPQQGYPPQQQQYPGQQGYPGQQQGYGPSQGG
PGPQYPNYPQGQGQQYGGYRPTQPGPPQPPQQRPYGYDQGQYGKYQQ
DNA sequence f or SS18
SEQ ID NO: 38
atgtctgtggctttcgcggccccgaggcagcgaggcaagggggagatcactcccgctgcgattcagaa
gatgttggatgacaataaccatcttattcagtgtataatggactctcagaataaaggaaagacctcag
agtgttctcagtatcagcagatgttgcacacaaacttggtataccttgctacaatagcagattctaat
caaaatatgcagtctcttttaccagcaccacccacacagaatatgcctatgggtcctggagggatgaa
tcagagcggccctcccccacctccacgctctcacaacatgccttcagatggaatggtaggtgggggtc
ctcctgcaccgcacatgcagaaccagatgaacggccagatgcctgggcctaaccatatgcctatgcag
ggacctggacccaatcaactcaatatgacaaacagttccatgaatatgccttcaagtagccatggatc
catgggaggttacaaccattctgtgccatcatcacagagcatgccagtacagaatcagatgacaatga
gtcagggacaaccaatgggaaaetatggtcccagaccaaatataagtatgcagccaaaccaaggtcca
atgatgcatcagcagcctccttctcagcaatacaatatgccacagggaggcggacagcattaccaagg
acagcagccacctatgggaatgatgggtcaagttaaccaaggcaatcatatgatgggtcagagacaga
ttcctccctatagacctcctcaacagggcccaccacagcagtactcaggccaggaagactattacggg
gaccaatacagtcatggtggacaaggtcctccagaaggcatgaaccagcaatattaccctgatggaaa
ttcacagtatggccaacagcaagatgcataccagggaccacctccacaacagggatatccaccccagc
agcagcagtacccagggcagcaaggttacecaggacagcagcagggctacggtccttcacagggtggt
ccaggtcctcagtatcctaactacccacagggacaaggtcagcagtatggaggatatagaccaacaca
gcctggaccaccacagccaccccagcagaggccttatggatatgaccagggacagtatggaaattacc
agcag
Protein sequence f or VPH
SEQ ID NO: 39
DALDDFDLDMLGSDALDDFDLDMLGSDALDDFDLDMLGSDALDDFDLDMLGSLPSASVEFEGSGGPSG
QISNQALALAPSSAPVLAQTMVPSSAMVPLAQPPAPAPVLTPGPPQSLSAPVPKSTQAGEGTLSEALL
HLQFDADEDLGALLGNSTDPGVFTDLASVDNSEFQQLLNQGVSMSHSTAEPMLMEYPEAITRLVTGSQ
RPPDpAPTPLGTSGLPNGLSGDEDFSSIADMDFSALLSQISSSGQGGGGSGFSVDTSALLDLFSPSVT
VPDNSLPDLDSSLASIQELLSPQEPPRPPEAENSSPDSGKQLVHYTAQPLFLLDPGSVDTGSNDLPVL
FELGESSYFSEGDGFAEDPTISLLTGSEPPKAKDPTVS
DNA sequence f or VPH
SEQ ID NO: 40
Gatgctttagacgattttgacttagatatgcttggttcagacgcgttagacgacttcgacctagacat
gttaggctcagatgcattggacgacttcgatttagatatgttgggctccgatgccctagatgactttg
atctagatatgctagggtcactacccagcgccagcgtcgagttcgaaggcagcggcgggccttcaggg
cagatcagcaaccaggccctggctctggcccctagctccgctccagtgctggcccagactatggtgcc
ctctagtgctatggtgcctctggcccagccacctgctccagcccctgtgctgaccccaggaccacccc
agtcactgagcgccccagtgcccaagtctacacaggccggcgaggggactctgagtgaagctctgctg
cacctgcagttcgacgctgatgaggacctgggagctctgctggggaacagcaccgatcccggagtgtt
cacagatctggcctccgtggacaactctgagtttcagcagctgctgaatcagagcgtgtccatgtctc
atagtacagccgaaccaatgctgatggagtaccccgaagccattacccggctggtgaccggcagccag
cggccccccgaccccgctccaactcccctgggaaccagcggcctgcctaatgggctgtccggagatga
agacttctcaagcatcgctgatatggactttagtgccctgctgtcacagatttcctctagtgggcagg
gaggaggtggaagcggcttcagcgtggacaccagtgccctgctggacctgttcagcccctcggtgacc
gtgcccgacatgagcctgcctgaccttgacagcagcctggccagtatccaagagctcctgtctcccca
ggagccccccaggcctcccgaggcagagaacagcagcccggattcagggaagcagctggtgcactaca
cagcgcagccgctgttcctgctggaccccggctccgtggacaccgggagcaacgacctgccggtgctg
tttgagctgggagagggctcctacttctccgaaggggacggcttcgccgaggaccccaccatctccct
gctgacaggctcggagcctcccaaagccaaggaccccactgtctcc
Protein sequence f or VPR
SEQ ID NO: 41
DALDDFDLDMLGSDALDDFDLDMLGSDALDDFDLDMLGSDALDDFDLDMLGSPKKKRKVGSQYLPDTD
DRHRIEEKRKRTYETFKSIMKKSPFSGPTDPRPPPRRIAVPSRSSASVPKPAPOPYPFTSSLSTINYD
EFPTMVFPSGOISQASALAPAPPQVLPQAPAPAPAPAMVSALAQAPAPVPVLAPGPPQAVAPPAPKPT
QAGEGTLSEALLOLQFDDEDLGALLGNSTDPAVFTDLASVDNSEFQOLLNQGIPVAPHTTEPMLMEYP
EAITRLVTGAQRPPDPAPAPLGAPGLPNGLLSGDEDFSSIADMDFSALLSQISSG5GSGSRD5REGMF
LPKPEAGSAISDVFEGREVCQPKRIRPFHPPGSPWANRPLPASLAPTPTGPVHEPVGSLTPAPVPQPL
DPAPAVTPEASHLLEDPDEETSQAVKALREMADTVIPQKEEAAICGQMDLSHPPPRGHLDELTTTLES
MTEDLNLDSPLTPELNEILDTFLNDECLLHAMHISTGLSIFDTSLF
DNA sequence f or VPR
SEQ ID NO: 42
gatgctttagacgattttgacttagatatgcttggttcagacgcgttagacgacttcgacctagacat
gttaggctcagatgcattggacgacttcgatttagatatgttgggctccgatgccctagatgactttg
atctagatatgctaggtagtcccaaaaagaagaggaaagtgggatcccagtatctgcccgacacagat
gatagacaccgaatcgaagagaaacgcaagcgaacgtatgaaaccttcaaatcgatcatgaagaaatc
gcccttctcgggtccgaccgatcccaggcccccaccgagaaggattgcggtcccgtcccgctcgtcgg
ccagcgtgccgaagcctgcgccgcagccctaccccttcacgtcgagcctgagcacaatcaattatgac
gagttcccgacgatggtgttcccctcgggacaaatctcacaagcctcggcgctcgcaccagcgcctcc
ccaagtccttccgcaagcgcctgccccagcgcctgcaccggcaatggtgtccgccctcgcacaggccc
ctgcgcccgtccccgtgctcgcgcctggaccgccccaggcggtcgctccaccggctccgaagccgacg
caggccggagagggaacactctccgaagcacttcttcaactccagtttgatgacgaggatcttggagc
actccttggaaactcgacagaccctgcggtgtttaccgacctcgcgtcagtagataactccgaatttc
agcagcttttgaaccagggtatcccggtcgcgccacatacaacggagcccatgttgatggaatacccc
gaagcaatcacgagacttgtgacgggagcgcagcggcctcccgatcccgcacccgcacctttgggggc
acctggcctccctaacggacttttgagcggcgacgaggatttctcctccatcgccgatatggatttct
cagccttgctgtcacagatttccagcggctctggcagcggcagccgggattccagggaagggatgttt
ttgccgaagcctgaggccggctccgctattagtgacgtgtttgagggccgcgaggtgtgccagccaaa
acgaatccggccatttcatcctccaggaagtccatgggccaaccgcccactccccgccagcctcgcac
caacaccaaccggtccagtacatgagccagtcgggtcactgaccccggcaccagtccctcagccactg
gatccagcgcccgcagtgactcccgaggccagtcacctgttggaggatcccgatgaagagacgagcca
ggctgtcaaagcccttcgggagatggccgatactgtgattccccagaaggaagaggctgcaatctgtg
gccaaatggacctttcccatccgcccccaaggggccatctggatgagctgacaaccacacttgagtcc
atgaccgaggatctgaacctggactcacccctgaccccggaattgaacgagattctggataccttcct

| Name | gRNA target/sequence | gRNA |
|---|---|---|
| gRNA1 | tagtcttagagtatccagtg | uagucuuagaguauccagug |
| promoter | (SEQ ID NO: 43) | (SEQ ID NO: 49) |
| HBG1/2 | | |
| gRNA2 | ggctagggatgaagaataaa | ggcuagggaugaagaauaaa |
| promoter | (SEQ ID NO: 44) | (SEQ ID NO: 50) |
| HBG1/2 | | |
| gRNA1 HS2 | aatatgtcacattctgtctc | aauaugucacauucugucuc |
| enhancer | (SEQ ID NO: 45) | {SEQ ID NO: 51) |
| HBG1/2 | | |
| gRNA2 HS2 | ggactatgggaggtcactaa | ggacuaugggaggucacuaa |
| enhancer | (SEQ ID NO: 46) | (SEQ ID NO: 52) |
| HBG1/2 | | |
| gRNA3HS2 | gaaggttacacagaaccaga | gaagguuacacagaaccaga |
| enhancer | (SEQ ID NO: 47) | (SEQ ID NO: 53) |
| HBG1/2 | | |
| gRNA4 HS2 | gccctgtaagcatcctgctg | gcCcuguaagcauccugcug |
| enhancer | (SEQ ID NO: 48) | (SEQ ID NO: 54) |
| HBG1/2 | | |

GS linker
SEQ ID NO: 55
(Gly-Gly-Gly-Gly-Ser)ₙ,
wherein n is an integer between 0 and 10
Linker
SEQ ID NO: 56
Gly-Gly-Gly-Gly-Gly
Linker
SEQ ID NO: 57
G1y-GIy-Ala-Gly Gly
Linker
SEQ ID NO: 58
Gly-G1y-Gly-Gly-Ser-Ser-Ser
Linker
SEQ ID NO: 59
Gly-Gly-Gly-Gly-Ala-Ala-Ala
SV40 NLS
SEQ ID NO: 60
Pro-Lys-Lys-Lys-Arg-Lys-Val
DNA sequence of the gRNA constant region
SEQ ID NO: 61
gtttaagagctatgctggaaacagcatagcaagtttaaataaggctagtccgttatcaactt
gaaaaagtggcaccgagtcggtgc
RNA sequence of the gRNA constant region
SEQ ID NO: 62
guuuaagagcuaugcuggaaacagcauagcaaguuuaaaaaaggcuaguccgauaacaacaa
gaaaaagaggcaccgagacggagc
DNA sequence for VPH-dCas9-SS18 (In backbone pNi36): pNi95; lowercase
underlined = VPH; capital underlined = dCas9; capital no underline = SS18.
SEQ ID NO: 63
atggatgctttagacgattttgacttagatatgcttggttcagacgcgttagacgacttcgacctaga
catgttaggctcagatgcattggacgacttcgatttagatatgttgggctccgatgccctagatgact
ttgatctagatatgctagggtcactacccagcgctagcgtcgagttcgaaggcagcggcgggccttca
gagcagatcagcaaccaggccctagctctggcccctagctcogctccagtgctggcccagactatggt
gccctctagtgctatggtgcctctggcccagccacctgctccagcccctgtgctgaccccaggaccac
cccagtcactgagcgctccagtgcccaagtctacacaggccggcgaggggactctgagtgaagctctg
ctgcacctgcagttcgacgctgatgaggacctgggagctctgctggggaacagcaccgatcccggagt
gttcacagatctggcctccgtggacaactctgagtttcagcagctgctgaatcagggcgtgtccatgt
ctcatagtacagccgaaccaatgctgatggagtaccccgaagccattacccggctggtgaccggcagc
cagcggccccccgaccccgctccaactcccctgggaaccagcggcctgcctaatgggctgtccggaga
tgaagacttctcaagcatcgctgatatggactttagtgccctgctgtcacagatttcctctagtgggc
agggaggaggtggaagcggcttcagcgtggacaccagtgccctgctggacctgttcagcccctcggtg
accgtgcccgacatgagcctgcctgaccttgacagcagcctggccagtatccaagagctcctatctcc
ccaggagccccccaggcctcccgaggcagagaacagcagcccggattcagggaagcagctggtgcact
acacagcgcagccgctgttcctgctggaccccggctccgtggacaccgggagcaacgacctgccggtg
ctgtttgagctgggagagggctcctacttctccgaaggggacggcttcgccgaggaccccaccatctc
cctgctgacaggctcggagcctcccaaagccaaggaccccactgtctccggctctggaggatctggcg
gctctagcgccaccATGGACAAGAAGTACAGCATCGGCCTGGCCATCGGCACCAACTCTGTGGGCTGG
GCCGTGATCACCGACGAGTACAAGGTGCCCAGCAAGAAATTCAAGGTGCTGGGCAACACCGACCGGCA
CAGCATCAAGAAGAAGAACCTCGGAGCCCTGCTGTTCGACAGCGGCGAAACAGCCGAGGCCACCCGGC
TGAAGAGAACCGCCAGAAGAAGATACACCAGACGGAAGAACCGGATCTGCTATCTGCAAGAGATCTTC
AGCAACGAGATGGCCAAGGTGGACGACAGCTTCTTCCACAGACTGGAAGAGTCCTTCCTGGTGGAAGA
GGATAAGAAGCACGAGCGGCACCCCATCTTCGGCAACATCGTGGACGAGGTGGCCTACCACGAGAAGT
ACCCCACCATCTACCACCTGAGAAAGAAACTGGTGGACAGCACCGACAAGGCCGACCTGCGGCTGATC
TATCTGGCCCTGGCCCACATGATCAAGTTCCGGGGCCACTTCCTGATCGAGGGCGACCTGAACCCCGA
CAACAGCGACGTGGACAAGCTGTTCATCCAGCTGGTGCAGACCTACAACCAGCTGTTCGAGGAAAACC
CCATCAACGCCAGCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCTGATTGCCCTGAG
CCTGGGCCTGACCCCCAACTTCAAGAGCAACTTCGACCTGGCCGAGGATGCCAAACTGCAGCTGAGCA
AGGACACCTACGACGACGACCTGGACAACCTGCTGGCCCAGATCGGCGACCAGTACGCCGACCTGTTT
CTGGCCGCCAAGAACCTGTCCGACGCCATCCTGCTGAGCGACATCCTGAGAGTGAACACCGAGATCAC
CAAGGCCCCCCTGAGCGCCTCTATGATCAAGAGATACGACGAGCACCACCAGGACCTGACCCTGCTGA
AAGCTCTCGTGCGGCAGCAGCTGCCTGAGAAGTACAAAGAGATTTTCTTCGACCAGAGCAAGAACGGC
TACGCCGGCTACATTGACGGCGGAGCCAGCCAGGAAGAGTTCTACAAGTTCATCAAGCCCATCCTGGA
AAAGATGGACGGCACCGAGGAACTGCTCGTGAAGCTGAACAGAGAGGACCTGCTGCGGAAGCAGCGGA
CCTTCGACAACGGLAGCATCCCCCACCAGATCCACCTGGGAGAGCTGCACGCCATTCTGCGGCGGCAG
GAAGATTTTTACCCATTCCTGAAGGACAACCGGGAAAAGATCGAGAAGATCCTGACCTTCCGCATCCC
CTACTACGTGGGCCCTCTGGCCAGGGGAAACAGCAGATTCGCCTGGATGACCAGAAAGAGCGAGGAAA
CCATCACCCCCTGGAACTTCGAGGAAGTGGTGGACAAGGGCGCTTCCGCCCAGAGCTTCATCGAGCGG
ATGACCAACTTCGATAAGAACCTGCCCAACGAGAAGGTGCTGCCCAAGCACAGCCTGCTGTACGAGTA
CTTCACCGTGTATAACGAGCTGACCAAAGTGAAATACGTGACCGAGGGAATGAGAAAGCCCGCCTTCC
TGAGCGGCGAGCAGAAAAAGGCCATCGTGGACCTGCTGTTCAAGACCAACCGGAAAGTGACCGTGAAG
CAGCTGAAAGAGGACTACTTCAAGAAAATCGAGTGCTTCGACTCCGTGGAAATCTCCGuCGTGGAAGA
TCGGTTCAACGCCTCCCTGGGCACATACCACGATCTGCTGAAAATTATCAAGGACAAGGACTTCCTGG
ACAATGAGGAAAACGAGGACATTCTGGAAGATATCGTGCTGACCCTGACACTGTTTGAGGACAGAGAG
ATGATCGAGGAACGGCTGAAAACCTATGCCCACCTGTTCGACGACAAAGTGATGAAGCAGCTGAAGCG
GCGGAGATACACCGGCTGGGGCAGGCTGAGCCGGAAGCTGATCAACGGCATCCGGGACAAGCAGTCCG
GCAAGACAATCCTGGATTTCCTGAAGTCCGACGGCTTCGCCAACAGAAACTTCATGCAGCTGATCCAC
GACGACAGCCTGACCTTTAAAGAGGACATCCAGAAAGCCCAGGTGTCCGGCCAGGGCGATAGCCTGCA
CGAGCACATTGCCAATCTGGCCGGCAGCCCCGCCATTAAGAAGGGCATCCTGCAGACAGTGAAGGTGG
TGGACGAGCTCGTGAAAGTGATGGGCCGGCACAAGCCCGAGAACATCGTGATCGAAATGGCCAGAGAG
AACCAGACCACCCAGAAGGGACAGAAGAACAGCCGCGAGAGAATGAAGCGGATCGAAGAGGGCATCAA
AGAGCTGGGCAGCCAGATCCTGAAAGAACACCCCGTGGAAAACACCCAGCTGCAGAACGAGAAGCTGT
ACCTGTACTACCTGCAGAATGGGCGGGATATGTACGTGGACCAGGAACTGGACATCAACCGGCTGTCC
GACTACGATGTGGACGCCATCGTGCCTCAGAGCTTTCTGAAGGACGACTCCATCGACAACAAGGTGCT
GACCAGAAGCGACAAGAACCGGGGCAAGAGCGACAACGTGCCCTCCGAAGAGGTCGTGAAGAAGATGA
AGAACTACTGGCGGCAGCTGCTGAACGCCAAGCTGATTACCCAGAGAAAGTTCGACAATCTGACCAAG
GCCGAGAGAGGCGGCCTGAGCGAACTGGATAAGGCCGGCTTCATCAAGAGACAGCTGGTGGAAACCCG
GCAGATCACAAAGCACGTGGCACAGATCCTGGACTCCCGGATGAACACTAAGTACGACGAGAATGACA
AGCTGATCCGGGAAGTGAAAGTGATCACCCTGAAGTCCAAGCTGGTGTCCGATTTCCGGAAGGATTTC
CAGTTTTACAAAGTGCGCGAGATCAACAACTACCACCACGCCCACGACGCCTACCTGAACGCCGTCGT
GGGAACCGCCCTGATCAAAAAGTACCCTAAGCTGGAAAGCGAGTTCGTGTACGGCGACTACAAGGTGT
ACGACGTGCGGAAGATGATCGCCAAGAGCGAGCAGGAAATCGGCAAGGCTACCGCCAAGTACTTCTTC
TACAGCAACATCATGAACTTTTTCAAGACCGAGATTACCCTGGCCAACGGCGAGATCCGGAAGCGGCC
TCTGATCGAGACAAACGGCGAAACCGGGGAGATCGTGTGGGATAAGGGCCGGGATTTTGCCACCGTGC
GGAAAGTGCTGAGCATGCCCCAAGTGAATATCGTGAAAAAGACCGAGGTGCAGACAGGCGGCTTCAGC
AAAGAGTCTATCCTGCCCAAGAGGAACAGCGATAAGCTGATCGCCAGAAAGAAGGACTGGGACCCTAA
GAAGTACGGCGGCTTCGACAGCCCCACCGTGGCCTATTCTGTGCTGGTGGTGGCCAAAGTGGAAAAGG
GCAAGTCCAAGAAACTGAAGAGTGTGAAAGAGCTGCTGGGGATCACCATCATGGAAAGAAGCAGCTTC
GAGAAGAATCCCATCGACTTTCTGGAAGCCAAGGGCTACAAAGAAGTGAAAAAGGACCTGATCATCAA
GCTGCCTAAGTACTCCCTGTTCGAGCTGGAAAACGGCCGGAAGAGAATGCTGGCCTCTGCCGGCGAAC
TGCAGAAGGGAAACGAACTGGCCCTGCCCTCCAAATATGTGAACTTCCTGTACCTGGCCAGCCACTAT
GAGAAGCTGAAGGGCTCCCCCGAGGATAATGAGCAGAAACAGCTGTTTGTGGAACAGCACAAGCACTA
CCTGGACGAGATCATCGAGCAGATCAGCGAGTTCTCCAAGAGAGTGATCCTGGCCGACGCTAATCTGG
ACAAAGTGCTGTCCGCCTACAACAAGCACCGGGATAAGCCCATCAGAGAGCAGGCCGAGAATATCATC
CACCTGTTTACCCTGACCAATCTGGGAGCCCCTGCCGCCTTCAAGTACTTTGACACCACCATCGACCG
GAAGAGGTACACCAGCACCAAAGAGGTGCTGGACGCCACCCTGATCCACCAGAGCATCACCGGCCTGT
ACGAGACACGGATCGACCTGTCTCAGCTGGGAGGCGACaagcgacctgccgccacaaagaaggctgga
caggctaagaagaagaaactggactctggaggatccgactacaaagaccatgacggtgattataaaga
tcatgacatcgattacaaggatgacgatgacaagggaggatccaaggagaagagtgcttgtcctaaag
atccagccaaacctccggccaaggcacaagttgtgggatggccaccggtgagatcataccggaagaac
gtgatggtttcctgccaaaaaccaagcggtggcccggaggcggcggcgctcgtgaaggtatcaatgga
cggagcaccgtacttgaggaaaatcgatttgaggatgtataaaggcggarctggcggctctggaggat
ccagcATGTCTGTGGCTTTCGCGGCCCCGAGGCAGCGAGGCAAGGGGGAGATCACTCCCGCTGCGATT
CAGAAGATGTTGGATGACAATAACCATCTTATTCAGTGTATAATGGACTCTCAGAATAAAGGAAAGAC
CTCAGAGTGTTCTCAGTATCAGCAGATGTTGCACACAAACTTGGTATACCTTGCTACAATAGCAGATT
CTAATCAAAATATGCAGTCTCTTTTACCAGCACCACCCACACAGAATATGCCTATGGGTCCTGGAGGG
ATGAATCAGAGCGGCCCTCCCCCACCTCCACGCTCTCACAACATGCCTTCAGATGGAATGGTAGGTGG
GGGTCCTCCTGCACCGCACATGCAGAACCAGATGAACGGCCAGATGCCTGGGCCTAACCATATGCCTA
TGCAGGGACCTGGACCCAATCAACTCAATATGACAAACAGTTCCATGAATATGCCTTCAAGTAGCCAT
GGATCCATGGGAGGTTACTACCATTCTGTGCCATCATCACAGAGCATGCCAGTACAGAATCAGATGAC
AATGAGTCAGGGACAACCAATGGGAAACTATGGTCCCAGACCAAATATGAGTATGCAGCCAAACCAAG
GTCCAATGATGCATCAGCAGCCTCCTTCTCAGCAATACAATATGCCACAGGGAGGCGGACAGCATTAC
CAAGGACAGCAGCCACCTATGGGAATGATGGGTCAAGTTAACCAAGGCAATCATATGATGGGTCAGAG
ACAGATTCCTCCCTATAGACCTCCTCAACAGGGCCCACCACAGCAGTACTCAGGCCAGGAAGACTATT
ACGGGGACCAATACAGTCATGGTGGACAAGGTCCTCCAGAACGCATGAACCAGCAATATTACCCTGAT
GGAAATTCACAGTATGGCCAACAGCAACATGCATACCAGGCACCACCTCCACAACAGGGATATCCACC
CCAGCACCAGCAGTACCCAGCGCAGCAAGGTTACCCAGGACAGCAGCAGGGCTACCGTCCTTCACAGG
GTGGTCCAGGTCCTCAGTATCCTAACTACCCACAGGGACAAGGTCAGCAGTATGGAGGATATAGACCA
ACACAGCCTGGACCACCACAGCCACCCCAGCAGAGGCCTTATGGATATGACCAGGGACAGTATGGAAA
TTACCAGCAGTGA
Amino acid sequence for VPH-dCas9-SS18
(corresponding to SEQ ID NO: 63); lowercase
underlined = VPH; capital underlined = dCas9;
capital no underline = SS18.
SEQ ID NO: 64
dalddfdldmlgsdalddfdldmlgsdalddfdldmlgsdalddfdldmlqslpsasvefegsgqpsg
qisngalalapssapvlagtmvpssamvplagppapapvltpgppqslsapvpkstaagegllseall
hlqfdadedlgallgnstdpgvftdlasvdnsefqqllnqgvsmshstaepmlmeypeaitrlvtgsq
rppdpaptplgtsglpnglsgdedfssiadmdfsailsqisssgqqgggsgfsvdtsalldlfspsvt
vpdmslpdldsslasiqellspqepprppeaensspdsgkqlvhytaqplflldpgsvdtgsndlpvl
felqegsyfseqdgfaedptislllqseppkakdptvsqsggsggssatMDKKYSIGLAIGTNSVGWA
VITDEYKVPSKKFKVLGNTDRHSIKKNLIGALLFDSGETAEATRLKRTARRRYTRRKNRICYLQEIFS
NEMAKVDDSFFHRLEESFLVEEDKKHEKHPIFGNIVDEVAYHEKYPTIYHLRKKLVDSTDKADLRLIY
LALAHMIKFRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIGDQYADLFL
AAKNLSDAILLSDILRVNTEITKAPLSASMIKRYDEHHQRLTLLKALVRQQLPEKYKEIFFDQSKNGY
AGYIDGGASQEEFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRKVTVKQ
LKEDYFKKIECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGITIMERSSFE
KNPIDFLEAKGYKEVKKDLIIKLPKYSLFELENGRKRMLASAGELQKGNELALPSKYVNFLYLASHYE
KLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGDkrpaatkkagq
akkkkldsggsdykdhdgdykdhdidykddddkggskeksacpkdpakppakaqvvgwppvrsyrknv
mvscqkssggpeaaafvkvsmdgapylrkidlrmykggsggsggssMSVAFAAPRQRGKGEITPAAIQ
KMLDDNNHLIQCIMDSQNKGKTSECSQYQQMLHTNLVYLATIADSNQNMQSLLPAPPTQNMPMGPGGM
NQSGPPPPPRSHNMPSDGMVGGGPPAPHMQNQMNGQMPGPNHMPMQGPGPNQLNMTNSSMNMPSSSHG
SMGGYNHSVPSSQSMPVQNQMTMSQGQPMGNYGPRPNMSMQPNQGPMMHQQPPSQQYNMPQGGGQHYQ
GQQPPMGMMGQVNQGNHMMGQRQIPPYRPPQQGPPQQYSGQEDYYGDQYSHGGQGPPEGMNQQYYPDG
NSQYGQQQDAYQGPPPQQGYPPQQQQYPGQQGYPGQQQGYGPSQGGPGPQYPNYPQGQGQQYGGYRPT
QPGPPQPPQQRPYGYDQGQYGNYQQ*
DNA sequence for VPH-dCas9-S818 (in backbone pNI144): pNH65; lowercase
underlined = VPH; capital underlined = dCas9;
capital no underline = SS18.
SEQ ID NO: 65
atggactacaaagaccatgacggtgattataaagatcatgacatcgattacaaggatgacgatgacaa
gcacgttgatgctttaggcgattttgacttagatatgcttggttcagacgcgttagacgacttcgacc
tagacatgttaggctcagatgcattggacgacttcgatttagatatgttgggctccgatgccctagat
gactttgatctagatatgctagggtcactacccagcgccagcgtcgagttcgaaggcagcggcgggcc
ttcagggcagatcagcaaccaggccctggctctggcccctagctccgccccagtgctggcccagacta
tggtaccctctagtgctatggtgcctctggcccagccacctgctccagcccctgtgctgaccccagga
ccaccccagtcactgagcgccccagtgcccaagtctacacaggccggcgacgggactctgagtgaagc
tctgctgcacctgcagttcgacgctgatgaggacctgggagctctgctggggaacagcaccgatcccg
gagtgttcacagatctggcctccgtggacaactctgagtttcagcagctgctgaatcagggcgtgtcc
atgtctcatagtacagccgaaccaatgctgatggagtaccccgaagccattacccggctggtgaccgg
cagccagcggccccccgaccccgctccaactcccctgggaaccagcggcctgcctaatgggctgtccg
gagatgaagacttctcaagcatcgctgatatggactttagtgccctgctgtcacagatttoctctagt
gggcagggaggaggtggaagcggcttcagcgtggacaccagtgccctgctggacctgttcagcccctc
ggtgaccgtgcccgacatgagcctgcctgaccttgacagcagcctggccagtatccaagagctcctgt
ctccccaggagccccccaggcctcccgaggcagagaacagcagcccggattcagggaagcagctggtg
cactacacagcgcagccgctgttcctgctggaccccggctccgtggacaccgggagcaacgacctgcc
ggtgctgtttgagctgggagagggctcctacttctccgaaggggacggcttcgccgaggaccccacca
tctccctgctgacaggctcggagcctcccaaagccaaggaccccactgtctccaaccccaagaagaag
aggaaggtgggccgcggaATGGACAAGAAGTAGTCCATTGGGCTCGCCATCGGCACAAACAGCGTCGG
CTGGGCCGTCATTACGGACGAGTACAAGGTGCCGAGCAAAAAATTCAAAGTTCTGGGCAATACCGATC
GCCACAGCATAAAGAAGAACCTCATTGGCGCCCTCCTGTTCGACTCCGGGGAAACCGCCGAAGCCACG
CGGCTCAAAAGAACAGCACGGCGCAGATATACCCGCAGAAAGAATCGGATCTGCTACCTGCAGGAGAT
CTTTAGTAATGAGATGGCTAAGGTGGATGACTCTTTCTTCCATAGGCTGGAGGAGTCCTTTTTGGTGG
AGGAGGATAAAAAGCACGAGCGCCACCCAATCTTTGGCAATATCGTGGACGAGGTGGCGTACCATGAA
AAGTACCCAACCATATATCATCTGAGGAAGAAGCTTGTAGACAGTACTGATAAGGCTGACTTGCGGTT
GATCTATCTcGCGCTGGCGCATATGATCAAATTTCGGGGACACTTCCTCATCGAGGGGGACCTGAACC
CAGACAACAGCGATGTCGACAAACTCTTTATCCAACTGGTTCAGACTTACAATCAGCTTTTCGAAGAG
AACCCGATCAACGCATCCGGAGTTGACGCCAAAGCAATCCTGAGCGCTAGGCTGTCCAAATCCCGGCG
GCTCGAAAACCTCATCGCACAGCTCCCTGGGGAGAAGAAGAACGGCCTGTTTGGTAATCTTATCGCCC
TGTCACTCGGGCTGACCCCCAACTTTAAATCTAACTTCGACCTGGCCGAAGATGCCAAGCTTCAACTG
AGCAAAGACACCTACGATGATGATCTCGACAATCTGCTGGCCCAGATCGGCGACCAGTACGCAGACCT
TTTTTTGGCGGCAAAGAACCTGTCAGACGCCATTCTGCTGAGTGATATTCTGCGAGTGAACACGGAGA
TCACCAAAGCTCCGCTGAGCGCTAGTATGATCAAGCGCTATGATGAGCACCACCAAGACTTGACTTTG
CTGAAGGCCCTTGTCAGACAGCAACTGCCTGAGAAGTACAAGGAAATTTTCTTCGATCAGTCTAAAAA
TGGCTACGCCGGATACATTGACGGCGGAGCAAGCCAGGAGGAATTTTACAAATTTATTAAGCCCATCT
TGGAAAAAATGGACGGCACCGAGGAGCTGCTGGTAAAGCTTAACAGAGAAGATCTGTTGCGCAAACAG
CGCACTTTCGACAATGGAAGCATCCCCCACCAGATTCACCTGGGCGAACTGCACGCTATCCTCAGGCG
GCAAGAGGATTTCTACCCCTTTTTGAAAGATAACAGGGAAAAGATTGAGAAAATCCTCACATTTCGGA
TACCCTACTATGTAGGCCCCCTCGCCCGGGGAAATTCCAGATTCGCGTGGATGACTCGCAAATCAGAA
GAGACCATCACTCCCTGGAACTTCGAGGAAGTCGTGGATAAGGGGGCCTCTGCCCAGTCCTTCATCGA
AAGGATGACTAACTTTGATAAAAATCTGCCTAACGAAAAGGTGCTTCCTAAACACTCTCTGCTGTACG
AGTACTTCACAGTTTATAACGAGCTCACCAAGGTCAAATACGTCACAGAAGGGATGAGAAAGCCAGCA
TTCCTGTCTGGAGAGCAGAAGAAAGCTATCGTGGACCTCCTCTTCAAGACGAACCGGAAAGTTACCGT
GAAACAGCTCAAAGAAGACTATTTCAAAAAGATTGAATGTTTCGACTCTGTTGAAATCAGCGGAGTGG
AGGATCGCTTCAACGCATCCCTGGGAACGTATCACGATCTCCTGAAAATCATTAAAGACAAGGACTTC
CTGGACAATGAGGAGAACGAGGACATTCTTGAGGACATTGTCCTCACCCTTACGTTGTTTGAAGATAG
GGAGATGATTGAAGAACGCTTGAAAACTTACGCTCATCTCTTCGACGACAAAGTCATGAAACAGCTCA
AGAGGCGCCGATATACAGGATGGGGGCGGCTGTCAAGAAAACTGATCAATGGGATCCGAGACAAGCAG
AGTGGAAAGACAATCCTGGATTTTCTTAAGTCCGATGGATTTGCCAACCGGAACTTCATGCAGTTGAT
CCATGATGACTCTCTCACCTTTAAGGAGGACATCCAGAAAGCACAAGTTTCTGGCCAGGGGGACAGTC
TTCACGAGCACATCGCTAATCTTGCAGGTAGCCCAGCTATCAAAAAGGGAATACTGCAGACCGTTAAG
GTCGTGGATGAACTCGTCAAAGTAATGGGAAGGCATAAGCCCGAGAATATCGTTATCGAGATGGCCCG
AGAGAACCAAACTACCCAGAAGGGACAGAAGAACAGTAGGGAAAGGATGAAGAGGATTGAAGAGGGTA
TAAAAGAACTGGGGTCCCAAATCCTTAAGGAACACCCAGTTGAAAACACCCAGCTTCAGAATGAGAAG
CTCTACCTGTACTACCTGCAGAACGGCAGGGACATGTACGTGGATCAGGAACTGGACATCAATCGGCT
CTCCGACTACGACGTGGATGCCATCGTGCCCCAGTCTTTTCTCAAAGATGATTCTATTGATAATAAAG
TGTTGACAAGATCCGATAAAAATAGAGGGAAGAGTGATAACGTCCCCTCAGAAGAAGTTGTCAAGAAA
ATGAAAAATTATTGGCGGCAGCTGCTGAACGCCAAACTGATCACACAACGGAAGTTCGATAATCTGAC
TAAGGCTGAACGAGGTGGCCTGTCTGAGTTGGATAAAGCCGGCTTCATCAAAAGGCAGCTTGTTGAGA
CACGCCAGATCACCAAGCACGTGGCCCAAATTCTCGATTCACGCATGAACACCAAGTACGATGAAAAT
GACAAACTGATTCGAGAGGTGAAAGTTATTACTCTGAAGTCTAAGCTGGTCTCAGATTTCAGAAAGGA
CTTTCAGTTTTATAAGGTGAGAGAGATCAACAATTACCACCATGCGCATGATGCCTACCTGAATGCAG
TGGTAGGCACTGCACTTATCAAAAAATATCCCAAGCTTGAATCTGAATTTGTTTACGGAGACTATAAA
GTGTACGATGTTAGGAAAATGATCGCAAAGTCTGAGCAGGAAATAGGCAAGGCCACCGCTAAGTACTT
CTTTTACAGCAATATTATGAATTTTTTCAAGACCGAGATTACACTGGCCAATGGAGAGATTCGGAAGC
GACCACTTATCGAAACAAACGGAGAAACAGGAGAAATCGTGTGGGACAAGGGTAGGGATTTCGCGACA
GTCCGGAAGGTCCTGTCCATGCCGCAGGTGAACATCGTTAAAAAGACCGAAGTACAGACCGGAGGCTT
CTCCAAGGAAAGTATCCTCCCGAAAAGGAACAGCGACAAGCTGATCGCACGCAAAAAAGATTGGGACC
CCAAGAAATACGGCGGATTCGATTCTCCTACAGTCGCTTACAGTGTACTGGTTGTGGCCAAAGTGGAG
AAAGGGAAGTCTAAAAAACTCAAAAGCGTCAAGGAACTGCTGGGCATCACAATCATGGAGCGATCAAG
CTTCGAAAAAAACCCCATCGACTTTCTCGAGGCGAAAGGATATAAAGAGGTCAAAAAAGACCTCATCA
TTAAGCTTCCCAAGTACTCTCTCTTTGAGCTTGAAAACGGCCGGAAACGAATGCTCGCTAGTGCGGGC
GAGCTGCAGAAAGGTAACGAGCTGGCACTGCCCTCTAAATACGTTAATTTCTTGTATCTGGCCAGCCA
CTATGAAAAGCTCAAAGGGTCTCCCGAAGATAATGAGCAGAAGCAGCTGTTCGTGGAACAACACAAAC
ACTACCTTGATGAGATCATCGAGCAAATAAGCGAATTCTCCAAAAGAGTGATCCTCGCCGACGCTAAC
CTCGATAAGGTGCTTTCTGCTTACAATAAGCACAGGGATAAGCCCATCAGGGAGCAGGCAGAAAACAT
TATCCACTTGTTTACTCTGACCAACTTGGGCGCGCCTGCAGCCTTCAAGTACTTCGACACCACCATAG
ACAGAAAGCGGTACACCTCTACAAAGGAGGTCCTGGACGCCACACTGATTCATCAGTCAATTACGGGG
CTCTATGAAACAAGAATCGACCTCTCTCAGCTCGGTGGAGACAGCAGGGCTGACcccaagaagaagag
gaaggtggctagcATGTCTGTGGCTTTCGCGGCCCCGAGGCAGCGAGGCAAGGGGGAGATCACTCCCG
CTGCGATTCAGAAGATGTTGGATGACAATAACCATCTTATTCAGTGTATAATGGACTCTCAGAATAAA
GGAAAGACCTCAGAGTGTTCTCAGTATCAGCAGATGTTGCACACAAACTTGGTATACCTTGCTACAAT
AGCAGATTCTAATCAAAATATGCAGTCTCTTTTACCAGCACCACCCACACAGAATATGCCTATGGGTC
CTGGAGGGATGAATCAGAGCGGCCCTCCCCCACCTCCACGCTCTCACAACATGCCTTCAGATGGAATG
GTAGGTGGGGGTCCTCCTGCACCGCACATGCAGAACCAGATGAACGGCCAGATGCCTGGGCCTAACCA
TATGCCTATGCAGGGACCTGGACCCAATCAACTCAATATGACAAACAGTTCCATGAATATGCCTTCAA
GTAGCCATGGATCCATGGGAGGTTACAACCATTCTGTGCCATCATCACAGAGCATGCCAGTACAGAAT
CAGATGACAATGAGTCAGGGACAACCAATGGGAAACTATGGTCCCAGACCAAATATGAGTATGCAGCC
AAACCAAGGTCCAATGATGCATCAGCAGCCTCCTTCTCAGCAATACAATATGCCACAGGGAGGCGGAC
AGCATTACCAAGGACAGCAGCCACCTATGGGAATGATGGGTCAAGTTAACCAAGGCAATCATATGATG
GGTCAGAGACAGATTCCTCCCTATAGACCTCCTCAACAGGGCCCACCACAGCAGTACTCAGGCCAGGA
AGACTATTACGGGGACCAATACAGTCATGGTGGACAAGGTCCTCCAGAAGGCATGAACCAGCAATATT
ACCCTGATGGAAATTCACAGTATGGCCAACAGCAAGATGCATACCAGGGACCACCTCCACAACAGGGA
TATCCACCCCAGCAGCAGCAGTACCCAGGGCAGCAAGGTTACCCAGGACAGCAGCAGGGCTACGGTCC
TTCACAGGGTGGTCCAGGTCCTCAGTATCCTAACTACCCACAGGGACAAGGTCAGCAGTATGGAGGAT
ATAGACCAACACAGCCTGGACCACCACAGCCACCCCAGCAGAGGCCTTATGGATATGACCAGGGACAG
TATGGAAATTACCAGCAGTGA
Amino acid sequence for VPH-dCas9-SS18
(corresponding to SEQ ID NO: 65); lowercase
underlined = VPH; capital underlined = dCas9;
capital no underline = SS18.
SEQ ID NO: 66
mdykdhdqdykdhdidykddddkhvdalddfdldmlqsdalddfdldmlqsdalddfdldmlqsdald
dfdldmlqslpsasvefegsqqpsgqisnqalalapssapvlaqtmvpssamvplaqppapapvltpg
ppgslsapvpkstqagegtlseallhlqfdadedlgallqnstdpgvftdlasvdnsefqqllnqgvs
mshstaepmlmeypeaitrlvtqsqrppdpaptplqtsglpnglsgdedfssiadmdfsallsqisss
qqggggsgfsvdtsalldlfspsvtvpdmslpdldsslasiqellspqepprppeaensspdsgkqlv
hytaqplflldpgsvdtgsndlpvlfelgegsyfsegdgfaedptislltgseppkakdptvsnpkkk
rkvgrgMDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLVQTYNQLFEE
NPINASGVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGRLSRKLINGIRDKQ
SGKTILDFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENTQLQNEK
LYLYYLQNGRDMYVDQELDINRLSDYDVDAIVPQSFLKDDSIDNKVLTRSDKNRGKSDNVPSEEVVKK
MKNYWRQLLNAKLITQRKFDNLTKAERGGLSELDKAGFIKRQLVETRQITKHVAQILDSRMNTKYDEN
DKLIREVKVITLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWDKGRDFAT
VRKVLSMPQVNTVKKTEVQTGGFSKESILPKRNSDKLIARKKDWDPKKYGGFDSPTVAYSVLVVAKVE
KGKSKKLKSVKELLGITIMERSSFEKNPIRFLEAKGYKEVKKDLlIKLPKYSLFELENGRKRMLASAG
ELQKGNELALPSKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVLDATLIHQSITG
LYETRIDLSQLGGDSRADpkkkrkvasMSVAFAAPRQRGKGEITPAAIQKMLDDNNHLIQCIMDSQNK
GKTSECSQYQQMLHTNLVYLATIADSNQNMQSLLPAPPTONMPMGPGGMNQSGPPPPPRSHNMPSDGM
VGGGPPAPHMQNQMNGQMPGPNHMPMQGPGPNQLNMTNSSMNMPSSSHGSMGGYNHSVPSSQSMPVQN
QMTMSQGQPMGNYGPRPNMSMQPNQGPMMHQQPPSQQYNMPQGGGQHYQGQQPPMGMMGQVNQGNHMM
GQRQIPPYRPPQQGPPQQYSGQEDYYGDQYSHGGQGPPEGMNQQYYPDGNSQYGQQQDAYQGPPPQQG
YPPQQQQYPGQQGYPGQQQGYGPSQGGPGPQYPNYPQGQGQQYGGYRPTQPGPPQPPQQRPYGYDQGQ
YGNYQQ
DNA sequence for VPH-dCas9 (in backbone pNI36):
pNI114; lowercase underlined = VPH;
capital underlined = dCas9.
SEQ ID NO: 67
atggatgctttagacgattttgacttagatatgcttggttcagacgcgttagacgacttcgacctaga
catgttaggctcagatgcattggacgacttcgatttagatatgttgggctccgatgccctagatgact
ttgatctagatatgctagggtcactacccagcgccagcgtcgagttcgaaggcagcggcgggccttca
gggcagatcagcaaccaggccctggctctggcccctagctccgctccagtgctggcccagactatggt
gccctctagtgctatggtgcctctggcccagccacctgctccagcccctgtgctgaccccaggaccac
cccagtcactgagcgccccagtgcccaagtctacacaggccggcgaggggactctgagtgaagctctg
ctgcacctgcagttcgacgctgatgaggacctgggagctctgctggggaacagcaccgatcccggagt
gttcacagatctggcctccgtggacaactctgagtttcagcagctgctgaatcagggcgtgtccatgt
ctcatagtacagccgaaccaatgctgatggagtaccccgaagccattacccggctggtgaccggcagc
cagcggccccccgaccccgctccaactcccctgggaaccagcggcctgcctaatgggctgtccggaga
tgaagacttctcaagcatogctgatatggactttagtgccctgctgtcacagatttcctctagtgggc
agggaggaggtggaagcggcttcagcgtggacaccagtgccctgctggacctgttcagcccctcggtg
accgtgcccgacatgagcctgcctgaccttgacagcagcctggccagtatccaagagctcctgtctcc
ccaggagccccccaggcctcccgaggcagagaacagcagcccggattcagggaagcagctggtgcact
acacagcgcagccgctgttcctgctggaccccggctccgtggacaccgggagcaacgacctgccggtg
ctgtttgagctgggagagggctcctacttctccgaaggggacggcttcgccgaggaccccaccatctc
cctgctgacaggctcggagcctcccaaagccaaggaccccactgtctccggctctggaggatctggcg
gctctagcgccaccATGGACAAGAAGTACAGCATCGGCCTGGCCATCGGCACCAACTCTGTGGGCTGG
GCCGTGATCACCGACGAGTACAAGGTGCCCAGCAAGAAATTCAAGGTGCTGGGCAACACCGACCGGCA
CAGCATCAAGAAGAACCTGATCGGAGCCCTGCTGTTCGACAGCGGCGAAACAGCCGAGGCCACCCGGC
TGAAGAGAACCGCCAGAAGAAGATACACCAGACGGAAGAACCGGATCTGCTATCTGCAAGAGATCTTC
AGCAACGAGATGGCCAAGGTGGACGACAGCTTCTTCCACAGACTGGAAGAGTCCTTCCTGGTGGAAGA
GGATAAGAAGCACGAGCGGCACCCCATCTTCGGCAACATCGTGGACGAGGTGGCCTACCACGAGAAGT
ACCCCACCATCTACCACCTGAGAAAGAAACTGGTGGACAGCACCGACAAGGCCGACCTGCGGCTGATC
TATCTGGCCCTGGCCCACATGATCAAGTTCCGGGGCCACTTCCTGATCGAGGGCGACCTGAACCCCGA
CAACAGCGACGTGGACAAGCTGTTCATCCAGCTGGTGCAGACCTACAACCAGCTGTTCGAGGAAAACC
CCATCAACGCCAGCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCTGATTGCCCTGAG
CCTGGGCCTGACCCCCAACTTCAAGAGCAACTTCGACCTGGCCGAGGATGCCAAACTGCAGCTGAGCA
AGGACACCTACGACGACGACCTGGACAACCTGCTGGCCCAGATCGGCGACCAGTACGCCGACCTGTTT
CTGGCCGCCAAGAACCTGTCCGACGCCATCCTGCTGAGCGACATCCTGAGAGTGAACACCGAGATCAC
CAAGGCCCCCCTGAGCGCCTCTATGATCAAGAGATACGACGAGCACCACCAGGACCTGACCCTGCTGA
AAGCTCTCGTGCGGCAGCAGCTGCCTGAGAAGTACAAAGAGATTTTCTTCGACCAGAGCAAGAACGGC
TACGCCGGCTACATTGACGGCGGAGCCAGCCAGGAAGAGTTCTACAAGTTCATCAAGCCCATCCTGGA
AAAGATGGACGGCACCGAGGAACTGCTCGTGAAGCTGAACAGAGAGGACCTGCTGCGGAAGCAGCGGA
CCTTCGACAACGGCAGCATCCCCCACCAGATCCACCTGGGAGAGCTGCACGCCATTCTGCGGCGGCAG
GAAGATTTTTACCCATTCCTGAAGGACAACCGGGAAAAGATCGAGAAGATCCTGACCTTCCGCATCCC
CTACTACGTGGGCCCTCTGGCCAGGGGAAACAGCAGATTCGCCTGGATGACCAGAAAGAGCGAGGAAA
CCATCACCCCCTGGAACTTCGAGGAAGTGGTGGACAAGGGCGCTTCCGCCCAGAGCTTCATCGAGCGG
ATGACCAACTTCGATAAGAACCTGCCCAACGAGAAGGTGCTGCCCAAGCACAGCCTGCTGTACGAGTA
CTTCACCGTGTATAACGAGCTGACCAAAGTGAAATACGTGACCGAGGGAATGAGAAAGCCCGCCTTCC
TGAGCGGCGAGCAGAAAAAGGCCATCGTGGACCTGCTGTTCAAGACCAACCGGAAAGTGACCGTGAAG
CAGCTGAAAGAGGACTACTTCAAGAAAATCGAGTGCTTCGACTCCGTGGAAATCTCCGGCGTGGAAGA
TCGGTTCAACGCCTCCCTGGGCACATACCACGATCTGCTGAAAATTATCAAGGACAAGGACTTCCTGG
ACAATGAGGAAAACGAGGACATTCTGGAAGATATCGTGCTGACCCTGACACTGTTTGAGGACAGAGAG
ATGATCGAGGAACGGCTGAAAACCTATGCCCACCTGTTCGACGACAAAGTGATGAAGCAGCTGAAGCG
GCGGAGATACACCGGCTGGGGCAGGCTGAGCCGGAAGCTGATCAACGGCATCCGGGACAAGCAGTCCG
GCAAGACAATCCTGGATTTCCTGAAGTCCGACGGCTTCGCCAACAGAAACTTCATGCAGCTGATCCAC
GACGACAGCCTGACCTTTAAAGAGGACATCCAGAAAGCCCAGGTGTCCGGCCAGGGCGATAGCCTGCA
CGAGCACATTGCCAATCTGGCCGGCAGCCCCGCCATTAAGAAGGGCATCCTGCAGACAGTGAAGGTGG
TGGACGAGCTCGTGAAAGTGATGGGCCGGCACAAGCCCGAGAACATCGTGATCGAAATGGCCAGAGAG
AACCAGACCACCCAGAAGGGACAGAAGAACAGCCGCGAGAGAATGAAGCGGATCGAAGAGGGCATCAA
AGAGCTGGGCAGCCAGATCCTGAAAGAACACCCCGTGGAAAACACCCAGCTGCAGAACGAGAAGCTGT
ACCTGTACTACCTGCAGAATGGGCGGGATATGTACGTGGACGAGGAACTGGACATCAACCGGCTGTCC
GACTACGATGTGGACGCCATCGTGCCTCAGAGCTTTCTGAAGGACGACTCCATCGACAACAAGGTGCT
GACCAGAAGCGACAAGAACCGGGGCAAGAGCGACAACGTGCCCTCCGAAGAGGTCGTGAAGAAGATGA
AGAACTACTGGCGGCAGCTGCTGAACGCCAAGCTGATTACCCAGAGAAAGTTCGACAATCTGACCAAG
GCCGAGAGAGGCGGCCTGAGCGAACTGGATAAGGCCGGCTTCATCAAGAGACAGCTGGTGGAAACCCG
GCAGATCACAAAGCACGTGGCACAGATCCTGGACTCCCGGATGAACACTAAGTACGACGAGAATGACA
AGCTGATCCGGGAAGTGAAAGTGATCACCCTGAAGTCCAAGCTGGTGTCCGATTTCCGGAAGGATTTC
CAGTTTTACAAAGTGCGCGAGATCAACAACTACCACCACGCLCACGACGCCTACCTGAACGCCGTCGt
GGGAACCGCCCTGATCAAAAAGTACCCTAAGCTGGAAAGCGAGTTCGTGTACGGCGACTACAAGGTGT
ACGACGTGCGGAAGATGATCGCCAAGAGCGAGCAGGAAATCGGCAAGGCTACCGCCAAGTACTTCTTC
TACAGCAACATCATGAACTTTTTCAAGACCGAGATTACCCTGGCCAACGGCGAGATCCGGAAGCGGCC
TCTGATCGAGACAAACGGCGAAACCGGGGAGATCGTGTGGGATAAGGGCCGGGATTTTGCCACCGTGC
GGAAAGTGCTGAGCATGCCCCAAGTGAATATCGTGAAAAAGACCGAGGTGCAGACAGGCGGCTTCAGC
AAAGAGTCTATCCTGCCCAAGAGGAACAGCGATAAGCTGATCGCCAGAAAGAAGGACTGGGACCCTAA
GAAGTACGGCGGCTTCGACAGCCCCACCGTGGCCTATTCTGTGCTGGTGGTGGCCAAAGTGGAAAAGG
GCAAGTCCAAGAAACTGAAGAGTGTGAAAGAGCTGCTGGGGATCACCATCATGGAAAGAAGCAGCTTC
GAGAAGAATCCCATCGACTTTCTGGAAGCCAAGGGCTACAAAGAAGTGAAAAAGGACCTGATCATCAA
GCTGCCTAAGTACTCCCTGTTCGAGCTGGAAAACGGCCGGAAGAGAATGCTGGCCTCTGCCGGCGAAC
TGCAGAAGGGAAACGAACTGGCCCTGCCCTCCAAATATGTGAACTTCCTGTACCTGGCCAGCCACTAT
GAGAAGCTGAAGGGCTCCCCCGAGGATAATGAGCAGAAACAGCTGTTTGTGGAACAGCACAAGCACTA
CCTGGACGAGATCATCGAGCAGATCAGCGAGTTCTCCAAGAGAGTGATCCTGGCCGACGCTAATCTGG
ACAAAGTGCTGTCCGCCTAGAACAAGCACCGGGATAAGCCCATCAGAGAGCAGGCCGAGAATATCATC
CACCTGTTTACCCTGACCAATCTGGGAGCCCCTGCCGCCTTCAAGTACTTTGACACCACCATCGACCG
GAAGAGGTACACCAGCACCAAAGAGGTGCTGGACGCCACCCTGATCCACCAGAGCATCACCGGCCTGT
ACGAGACACGGATCGACCTGTCTCAGCTGGGAGGCGAC
Amino acid sequence for VPH-dCas9
(corresponding to SEQ ID NO: 67); lowercase
underlined = VPH; capital underlined = dCas9.
SEQ ID NO: 68
dalddfdldmlgsdalddfdldmlgsdalddfdldmlgsdalddfdldmlgslpsasvefegsggpsg
qisnqalalapssapvlaqtmvpssamvplaqppapapvltpgppqslsapvpkstqaqegllseall
hlqfdadedlgallgnstdpgvftdlasvdnsefgqlinqgvsmshstaepmlmeypeaitrlvtgsq
rppdpaptplgtsglpnglsgdedfssiadmdfsallsqisssgqggggsgfsvdtsalldlfspsvt
vpdmslpdldsslasiqellspqepprppeaensspdsgkqlvhytaqplflldpgsvdtgsndlpvl
felgeqsyfsegdgfaedptislltgseppkakdptvsgsqgsggssatMDKKYSIGLAIGTNSVGWA
VITDEYKVPSKKFKVLGNTDRHSIKKNLIGALLFDSGETAEATRLKRTARRRYTRRKNRICYLQEIFS
NEMAKVDDSFFHRLEESFLVEEDKKHERHPIFGNIVDEVAYHEKYPTIYHLRKKLVDSTDKADLRLIY
LALAHMIKFRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIGDQYADLFL
AAKNLSDAILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRKVTVKQ
LKEDYFKKIECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGITIMERSSFE
KNPIDFLEAKGYKEVKKDLIIKLPKYSLFELENGRKRMLASAGELQKGNELALPSKYVNFLYLASHYE
KLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD
DNA sequence for VPH-dCas9 (in backbone pNI123):
pNI136; lowercase underlined = VPH;
capital underlined = dCas9.
SEQ ID NO: 69
atggactacaaagaccacgacggCgattataaagatcacgacatcgattacaaggatgacgatgacaa
gcacgttgatgctttagacgattttgacttagatatgcttggttcagacgcgttagacgacttcgacc
tagacatgttaggctcagatgcattggacgacttcgatttagatatgttgggctccgatgccctagat
gactttgatctagatatgctagggtcactacccagcgccagcgtcgagttcgaaggcagcggcgggcc
ttcagggcagatcagcaaccaggccctggctctggcccctagctccgctccagtgctggcccagacta
tggtgccctctagtgctatggtgcctctggcccagccacctgctccagcccctgtgctgaccccagga
ccaccccagtcactgagcgccccagtgcccaagtctacacaggccggcgaggggactctgagtgaagc
tctgctgcacctgcagttcgacgctgatgaggacctgggagctctgctggggaacagcaccgatcccg
gagtgttcacagatctggcctccgtggacaactctgagtttcagcagctgctgaatcagggcgtgtcc
atgtctcatagtacagccgaaccaatgctgatggagtaccccgaagccattacccggctggtgaccgg
cagccagcggccccccgaccccgctccaactcccctgggaaccagcggcctgcctaatgggctgtccg
gagatgaagacttctcaagcatcgctgatatggactttagtgccctgctgtcacagatttcctctagt
gggcagggaggaggtggaagcggcttcagcgtggacaccagtgccctgctggacctgttcagcccctc
ggtgaccgtgcccgacatgagcctgcctgaccttgacagcagcctggccagtatccaagagctcctgt
ctccccaggagccccccaggcctcccgaggcagagaacagcagcccggattcagggaagcagctggtg
cactacacagcgcagccgctgttcctgctggaccccggctccgtggacaccgggagcaacgacctgcc
ggtgctgtttgagctgggagagggctcctacttctccgaaggggacggcttcgccgaggaccccacca
tctccctgctgacaggctcggagcctcccaaagccaaggaccccactgtctccaaccccaagaagaag
aggaaggtgggccgcggaATGGACAAGAAGTACTCCATTGGGCTCGCCATCGGCACAAACAGCGTCGG
CTGGGCCGTCATTACGGACGAGTACAAGGTGCCGAGCAAAAAATTCAAAGTTCTGGGCAATACCGATC
GCCACAGCATAAAGAAGAACCTCATTGGCGCCCTCCTGTTCGACTCCGGGGAAACCGCCGAAGCCACG
CGGCTCAAAAGAACAGCACGGCGCAGATATACCCGCAGAAAGAATCGGATCTGCTACCTGCAGGAGAT
CTTTAGTAATGAGATGGCTAAGGTGGATGACTCTTTCTTCCATAGGCTGGAGGAGTCCTTTTTGGTGG
AGGAGGATAAAAAGCACGAGCGCCACCCAATCTTTGGCAATATCGTGGACGAGGTGGCGTACCATGAA
AAGTACCCAACCATATATCATCTGAGGAAGAAGCTTGTAGACAGTACTGATAAGGCTGACTTGCGGTT
GATCTATCTCGCGCTGGCGCATATGATCAAATTTCGGGGACACTTCCTCATCGAGGGGGACCTGAACC
CAGACAACAGCGATGTCGACAAACTCTTTATCCAACTGGTTCAGACTTACAATCAGCTTTTCGAAGAG
AACCCGATCAACGCATCCGGAGTTGACGCCAAAGCAATCCTGAGCGCTAGGCTGTCCAAATCCCGGCG
GCTCGAAAACCTCATCGCACAGCTCCCTGGGGAGAAGAAGAACGGCCTGTTTGGTAATCTTATCGCCC
TGTCACTCGGGCTGACCCCCAACTTTAAATCTAACTTCGACCTGGCCGAAGATGCCAAGCTTCAACTG
AGCAAAGACACCTACGATGATGATCTCGACAATCTGCTGGCCCAGATCGGCGACCAGTACGCAGACCT
TTTTTTGGCGGCAAAGAACCTGTCAGACGCCATTCTGCTGAGTGATATTCTGCGAGTGAACACGGAGA
TCACCAAAGCtCCGCTGAGCGCTAGTATGATCAAGCGCTATGATGAGCACCACCAAGACTTGACTTTG
CTGAAGGCCCTTGTCAGACAGCAACTGCCTGAGAAGTACAAGGAAATTTTCTTCGATCAGTCTAAAAA
TGGCTACGCCGGATACATTGACGGCGGAGCAAGCCAGGAGGAATTTTACAAATTTATTAAGCCCATCT
TGGAAAAAATGGACGGCACCGAGGAGCTGCTGGTAAAGCTTAACAGAGAAGATCTGTTGCGCAAACAG
CGCACTTTCGACAATGGAAGCATCCCCCACCAGATTCACCTGGGCGAACTGCACGCTATCCTCAGGCG
GCAAGAGGATTTCTACCCCTTTTTGAAAGATAACAGGGAAAAGATTGAGAAAATCCTCACATTTCGGA
TACCCTACTATGTAGGCCCCCTCGCCCGGGGAAATTCCAGATTCGCGTGGATGACTCGCAAATCAGAA
GAGACCATCACTCCCTGGAACTTCGAGGAAGTCGTGGATAAGGGGGCCTCTGCCCAGTCCTTCATCGA
AAGGATGACTAACTTTGATAAAAATCTGCCTAACGAAAAGGTGCTTCCTAAACACTCTCTGCTGTACG
AGTACTTCACAGTTTATAACGAGCTCACCAAGGTCAAATACGTCACAGAAGGGATGAGAAAGCCAGCA
TTCCTGTCTGGAGAGCAGAAGAAAGCTATCGTGGACCTCCTCTTCAAGACGAACCGGAAAGTTACCGT
GAAACAGCTCAAAGAAGACTATTTCAAAAAGATTGAATGTTTCGACTCTGTTGAAATCAGCGGAGTGG
AGGATCGCTTCAACGCATCCCTGGGAACGTATCACGATCTCCTGAAAATCATTAAAGACAAGGACTTC
CTGGACAATGAGGAGAACGAGGACATTCTTGAGGACATTGTCCTCACCCTTACGTTGTTTGAAGATAG
GGAGATGATTGAAGAACGCTTGAAAACTTACGCTCATCTCTTCGACGACAAAGTCATGAAACAGCTCA
AGAGGCGCCGATATACAGGATGGGGGCGGCTGTCAAGAAAACTGATCAATGGGATCCGAGACAAGCAG
AGTGGAAAGACAATCCTGGATTTTCTTAAGTCCGATGGATTTGCCAACCGGAACTTCATGCAGTTGAT
CCATGATGACTCTCTCACCTTTAAGGAGGACATCCAGAAAGCACAAGTTTCTGGCCAGGGGGACAGTC
TTCACGAGCACATCGCTAATCTTGCAGGTAGCCCAGCTATCAAAAAGGGAATACTGCAGACCGTTAAG
GTCGTGGATGAACTCGTCAAAGTAATGGGAAGGCATAAGCCCGAGAATATCGTTATCGAGATGGCCCG
AGAGAACCAAACTACCCAGAAGGGACAGAAGAACAGTAGGGAAAGGATGAAGAGGATTGAAGAGGGTA
TAAAAGAACTGGGGTCCCAAATCCTTAAGGAACACCCAGTTGAAAACACCCAGCTTCAGAATGAGAAG
CTCTACCTGTACTACCTGCAGAACGGCAGGGACATGTACGTGGATCAGGAACTGGACATCAATCGGCT
CTCCGACTACGACGTGGATGCCATCGTGCCCCAGTCTTTTCTCAAAGATGATTCTATTGATAATAAAG
TGTTGACAAGATCCGATAAAAATAGAGGGAAGAGTGATAACGTCCCCTCAGAAGAAGTTGTCAAGAAA
ATGAAAAATTATTGGCGGCAGCTGCTGAACGCCAAACTGATCACACAACGGAAGTTCGATAATCTGAC
TAAGGCTGAACGAGGTGGCCTGTCTGAGTTGGATAAAGCCGGCTTCATCAAAAGGCAGCTTGTTGAGA
CACGCCAGATCACCAAGCACGTGGCCCAAATTCTCGATTCACGCATGAACACCAAGTACGATGAAAAT
GACAAACTGATTCGAGAGGTGAAAGTTATTACTCTGAAGTCTAAGCTGGTCTCAGATTTCAGAAAGGA
CTTTCAGTTTTATAAGGTGAGAGAGATCAACAATTACCACCATGCGCATGATGCCTACCTGAATGCAG
TGGTAGGCACTGCACTTATCAAAAAATATCCCAAGCTTGAATCTGAATTTGTTTACGGAGACTATAAA
GTGTACGATGTTAGGAAAATGATCGCAAAGTCTGAGCAGGAAATAGGCAAGGCCACCGCTAAGTACTT
CTTTTACAGCAATATTATGAATTTTTTCAAGACCGAGATTACACTGGCCAATGGAGAGATTCGGAAGC
GACCACTTATCGAAACAAACGGAGAAACAGGAGAAATCGTGTGGGACAAGGGTAGGGATTTCGCGACA
GTCCGGAAGGTCCTGTCCATGCCGCAGGTGAACATCGTTAAAAAGACCGAAGTACAGACCGGAGGCTT
CTCCAAGGAAAGTATCCTCCCGAAAAGGAACAGCGACAAGCTGATCGCACGCAAAAAAGATTGGGACC
CCAAGAAATACGGCGGATTCGATTCTCCTACAGTCGCTTACAGTGTACTGGTTGTGGCCAAAGTGGAG
AAAGGGAAGTCTAAAAAACTCAAAAGCGTCAAGGAACTGCTGGGCATCACAATCATGGAGCGATCAAG
CTTCGAAAAAAACCCCATCGACTTTCTCGAGGCGAAAGGATATAAAGAGGTCAAAAAAGACCTCATCA
TTAAGCTTCCCAAGTACTCTCTCTTTGAGCTTGAAAACGGCCGGAAACGAATGCTCGCTAGTGCGGGC
GAGCTGCAGAAAGGTAACGAGCTGGCACTGCCCTCTAAATACGTTAATTTCTTGTATCTGGCCAGCCA
CTATGAAAAGCTCAAAGGGTCTCCCGAAGATAATGAGCAGAAGCAGCTGTTCGTGGAACAACACAAAC
ACTACCTTGATGAGATCATCGAGCAAATAAGCGAATTCTCCAAAAGAGTGATCCTCGCCGACGCTAAC
CTCGATAAGGTGCTTTCTGCTTACAATAAGCACAGGGATAAGCCCATCAGGGAGCAGGCAGAAAACAT
TATCCACTTGTTTACTCTGACCAACTTGGGCGCGCCTGCAGCCTTCAAGTACTTCGACACCACCATAG
ACAGAAAGCGGTACACCTCTACAAAGGAGGTCCTGGACGCCACACTGATTCATCAGTCAATTACGGGG
CTCTATGAAACAAGAATCGACCTCTCTCAGCTCGGTGGAGACAGCAGGGCTGAC
Amino acid sequence for VPH-dCas9
(corresponding to SEQ ID NO: 69); lowercase
underlined = VPH; capital underlined = dCas9
dykdhdgdykdhdidykddddkhvdalddfdldmlgsdalddfdldmlgsdalddfdldmlgsdaldd
SEQ ID NO: 70
fdldmlgslpsasvefegsggpsgaisngalalapssapvlaqtmvpssamvplaqppapapvltpgp
pqslsapvpkstqagegtlseallhlqfdadedlgallgnstdpgvftdlasydnsefqqllnqgvsm
shstaepmlmeypeaitrlvtgsqrppdpaptplqtsglpnqlsgdedfssiadmdfsailsqisssg
qggggsqfsvdtsalldlfspsvtvpdmslpdldsslasiqellspqepprppeaensspdsqkqlvh
ytaqplflldpgsvdtgsndlpvlfelgegsyfsegdgfaedptislllqseppkakdptvsnpkkkr
kvgrgMDKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFAWMTRKSEE
TITPWNFEEVVDKGASAQSFTERMTNFDKNLPNEKVLPKHSLLYEYFTVYNELTKVKYVTEGMRKPAE
LSGEQKKAIVDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQILKEHPVENTQLQNEKL
YLYYLQNGRDMYVDQELDINRLSDYDVDAIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNTKYDEND
KLIREVKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSKRVILADANL
DKVLSAYNKHRDKPIREQAENIIHLFTLTNLGAPAAFKYFDTTIDRKRYTSTKEVLDATLIHQSITGL
YETRIDLSQLGGDSRAD
DNA sequence for dCas9-VPH (in backbone pN/36):
pNI70; lowercase underlined = VPH;
capital underlined = dCas9.
SEQ ID NO: 71
ATGGACAAGAAGTACAGCATCGGCCTGGCCATCGGCACCAACTCTGTGGGCTGGGCCGTGATCACCGA
CGAGTACAAGGTGCCCAGCAAGAAATTCAAGGTGCTGGGCAACACCGACCGGCACAGCATCAAGAAGA
ACCTGATCGGAGCCCTGCTGTTCGACAGCGGCGAAACAGCCGAGGCCACCCGGCTGAAGAGAACCGCC
AGAAGAAGATACACCAGACGGAAGAACCGGATCTGCTATCTGCAAGAGATCTTCAGCAACGAGATGGC
CAAGGTGGACGACAGCTTCTTCCACAGACTGGAAGAGTCCTTCCTGGTGGAAGAGGATAAGAAGCACG
AGCGGCACCCCATCTTCGGCAACATCGTGGACGAGGTGGCCTACCACGAGAAGTACCCCACCATCTAC
CACCTGAGAAAGAAACTGGTGGACAGCACCGACAAGGCCGACCTGCGGCTGATCTATCTGGCCCTGGC
CCACATGATCAAGTTCCGGGGCCACTTCCTGATCGAGGGCGACCTGAACCCCGACAACAGCGACGTGG
ACAAGCTGTTCATCCAGCTGGTGCAGACCTACAACCAGCTGTTCGAGGAAAACCCCATCAACGCCAGC
GGCGTGGACGCCAAGGCCATCCTGTCTGCCAGACTGAGCAAGAGCAGACGGCTGGAAAATCTGATCGC
CCAGCTGCCCGGCGAGAAGAAGAATGGCCTGTTCGGAAACCTGATTGCCCTGAGCCTGGGCCTGACCC
CCAACTTCAAGAGCAACTTCGACCTGGCCGAGGATGCCAAACTGCAGCTGAGCAAGGACACCTACGAC
GACGACCTGGACAACCTGCTGGCCCAGATCGGCGACCAGTACGCCGACCTGTTTCTGGcCGCCAAGAA
CCTGTCCGACGCCATCCTGCTGAGCGACATCCTGAGAGTGAACACCGAGATCACCAAGGCCCCCCTGA
GcGCCTCTATGATCAAGAGATACGACGAGCACCACCAGGACCTGACCCTGCTGAAAGCTCTCGTGcGG
CAGCAGCTGCCTGAGAAGTACAAAGAGATTTTCTTCGACCAGAGCAAGAACGGCTACGCCGGCTACAT
TGACGGCGGAGCCAGCCAGGAAGAGTTCTACAAGTTCATCAAGCCCATCCTGGAAAAGATGGACGGCA
CCGAGGAACTGCTCGTGAAGCTGAACAGAGAGGACCTGCTGCGGAAGCAGCGGACCTTCGACAACGGC
AGCATCCCCCACCAGATCCACCTGGGAGAGCTGCACGCCATTCTGCGGCGGCAGGAAGATTTTTACCC
ATTCCTGAAGGACAACCGGGAAAAGATCGAGAAGATCCTGACCTTCCGCATCCCCTAGTAGGTGGGCC
CTCTGGCCAGGGGAAACAGCAGATTCGCCTGGATGACCAGAAAGAGCGAGGAAACCATCACCCCCTGG
AACTTCGAGGAAGTGGTGGACAAGGGCGCTTCCGCCCAGAGCTTCATCGAGCGGATGACCAACTTCGA
TAAGAACCTGCCCAACGAGAAGGTGCTGCCCAAGCACAGCCTGCTGTACGAGTACTTCACCGTGTATA
ACGAGCTGACCAAAGTGAAATACGTGACCGAGGGAATGAGAAAGCCCGCCTTCCTGAGCGGCGAGCAG
AAAAAGGCCATCGTGGACCTGCTGTTCAAGACCAACCGGAAAGTGACCGTGAAGCAGCTGAAAGAGGA
CTACTTCAAGAAAATCGAGTGCTTCGACTCCGTGGAAATCTCCGGCGTGGAAGATCGGTTCAACGCCT
CCCTGGGCACATACCACGATCTGCTGAAAATTATCAAGGACAAGGACTTCCTGGACAATGAGGAAAAC
GAGGACATTCTGGAAGATATCGTGCTGACCCTGACACTGTTTGAGGACAGAGAGATGATCGAGGAACG
GCTGAAAACCTATGCCCACCTGTTCGACGACAAAGTGATGAAGCAGCTGAAGCGGCGGAGATACACCG
GCTGGGGCAGGCTGAGCCGGAAGCTGATCAACGGCATCCGGGACAAGCAGTCCGGCAAGACAATCCTG
GATTTCCTGAAGTCCGACGGCTTCGCCAACAGAAACTTCATGCAGCTGATCCACGACGACAGCCTGAC
CTTTAAAGAGGACATCCAGAAAGCCCAGGTGTCCGGCCAGGGCGATAGCCTGCACGAGCACATTGCCA
ATCTGGCCGGCAGCCCCGCCATTAAGAAGGGCATCCTGCAGACAGTGAAGGTGGTGGACGAGCTCGTG
AAAGTGATGGGCCGGCACAAGCCCGAGAACATCGTGATCGAAATGGCCAGAGAGAACCAGACCACCCA
GAAGGGACAGAAGAACAGCCGCGAGAGAATGAAGCGGATCGAAGAGGGCATCAAAGAGCTGGGCAGCC
AGATCCTGAAAGAACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGACTACGATGTGGA
CGCCATCGTGCCTCAGAGCTTTCTGAAGGACGACTCCATCGACAACAAGGTGCTGACCAGAAGCGACA
AGAACCGGGGCAAGAGCGACAACGTGCCCTCCGAAGAGGTCGTGAAGAAGATGAAGAACTACTGGCGG
CAGCTGLTGAACGCCAAGCTGATTACCCAGAGAAAGTTCGACAATCTGACCAAGGCCGAGAGAGGCGG
CCTGAGCGAACTGGATAAGGCCGGCTTCATCAAGAGACAGCTGGTGGAAACCCGGLAGATCACAAAGC
ACGTGGCACAGATCCTGGACTCCCGGATGAACACTAAGTACGACGAGAATGACAAGCTGATCCGGGAA
GTGAAAGTGATCACCCTGAAGTCCAAGCTGGTGTCCGATTTCCGGAAGGATTTCCAGTTTTACAAAGT
GCGCGAGATCAACAACTACCACCACGCCCACGACGCCTACCTGAACGCCGTCGTGGGAACCGCCCTGA
TCAAAAAGTACCCTAAGCTGGAAAGCGAGTTCGTGTACGGCGACTACAAGGTGTACGACGTGCGGAAG
ATGATCGCCAAGAGCGAGCAGGAAATCGGCAAGGCTACCGCCAAGTACTTCTTCTACAGCAACATCAT
GAACTTTTTCAAGACCGAGATTACCCTGGCCAACGGCGAGATCCGGAAGCGGCCTCTGATCGAGACAA
ACGGCGAAACCGGGGAGATCGTGTGGGATAAGGGCCGGGATTTTGCCACCGTGCGGAAAGTGCTGAGC
ATGCCCCAAGTGAATATCGTGAAAAAGACCGAGGTGCAGACAGGCGGCTTCAGCAAAGAGTCTATCCT
GCCCAAGAGGAACAGCGATAAGCTGATCGCCAGAAAGAAGGACTGGGACCCTAAGAAGTACGGCGGCT
TCGACAGCCCCACCGTGGCCTATTCTGTGCTGGTGGTGGCCAAAGTGGAAAAGGGCAAGTCCAAGAAA
CTGAAGAGTGTGAAAGAGCTGCTGGGGATCACCATCATGGAAAGAAGCAGCTTCGAGAAGAATCCCAT
CGACTTTCTGGAAGCCAAGGGCTACAAAGAAGTGAAAAAGGACCTGATCATCAAGCTGCCTAAGTACT
CCCTGTTCGAGCTGGAAAACGGCCGGAAGAGAATGCTGGCCTCTGCCGGCGAACTGCAGAAGGGAAAC
GAACTGGCCCTGCCCTCCAAATATGTGAACTTCCTGTACCTGGCCAGCCACTATGAGAAGCTGAAGGG
CTCCCCCGAGGATAATGAGCAGAAACAGCTGTTTGTGGAACAGCACAAGCACTACCTGGACGAGATCA
TCGAGCAGATCAGCGAGTTCTCCAAGAGAGTGATCCTGGCCGACGCTAATCTGGACAAAGTGCTGTCC
GCCTACAACAAGCACCGGGATAAGCCCATCAGAGAGCAGGCCGAGAATATCATCCACCTGTTTACCCT
GACCAATCTGGGAGCCCCTGCCGCCTTCAAGTACTTTGACACCACCATCGACCGGAAGAGGTACACCA
GCACCAAAGAGGTGCTGGACGCCACCCTGATCCACCAGAGCATCACCGGCCTGTACGAGACACGGATC
GACCTGTCTCAGCTGGGAGGCGACaagcgacctgccgccacaaagaagqctggacaggctaagaagaa
gaaactggactctggaggatccgactacaaagaccatgacggtgattataaagatcatgacatcgatt
acaaggatgacgatgacaagggaggatccaaggagaagagtgcttgtcctaaagatccagccaaacct
ccggccaaggcacaagttgtgggatggccaccggtgagatcataccggaagaacgtgatggtttcctg
ccaaaaatcaagcggtggcccggaggcggcggcgttcgtgaaggtatcaatggacggagcaccgtact
tgaggaaaatcgatttgaggatgtataaaggcggatctggcggctctggaggatccagcgatgcttta
gacgattttgacttagatatgcttggttcagacgcgttagacgacttcgacctagacatgttaggctc
agatgcattggacgacttcgatttagatatgttgggctccgatgccctagatgactttgatctagata
tgctagggtcactacccagcgctagcgtcgagttcgaaggcagcggcgggccttcagggcagatcagc
aaccaggccctggctctggcccctagctccgctccagtgctggcccagactatggtgccctctagtgc
tatggtgcctctggcccagccacctgctccagcccctgtgctgaccccaggaccaccccagtcactga
gcgctccagtgcccaagtctacacaggccggcgaggggactctgagtgaagctctgctgcacctgcag
ttcgacgctgatgaggacctgggagctctgctggggaacagcaccgatcccggagtgttcacagatct
ggcctccgtggacaactctgagtttcagcagctgctgaatcagggcgtgtccatgtctcatagtacag
ccgaaccaatgctgatggagtaccccgaagccattacccggctggtgaccggcagccagcggcccccc
gaccccgctccaactcccctgggaaccageggcctgcctaatgggctatcoggagatgaagacttctc
aagcatcgctgatatggactttagtgccctgctgtcacagatttcctctagtgggcagggaggaggtg
gaagcggcttcagcgtggacaccagtgccctgctggacctgttcagcccctcggtgaccgtgcccgac
atgagcctgcctgaccttgacagcagcctggccagtatccaagagctcctgtctccccaggagccccc
caggcctcccgaggcagagaacagcagcccggattcagggaagcagctggtgcactacacagcgcagc
cgctgttcctgctggaccccggctccgtggacaccgggagcaacgacctgccggtgctgtttgagctg
ggagagggctcctacttctccgaaggggacggcttcgccgaggaccccaccatctccctgctgacagg
ctcggagcctcccaaagccaaggaccccactgtctcctga
Amino acid sequence fordCas9-VPH
(corresponding to SEQ ID NO: 71); lowercase
underlined = VPH; capital underlined = dCas9.
SEQ ID NO: 72
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDkrpaatkkagqakkkkldsggsdykdhdgdykdhdidykddddkggskeksacpkdpakp
pakaqvvgwppvrsyrknvmvscqkssggpeaaafvkvsmdgapylrkidlrmykggsggsggssdal
ddfdldmlgsdalddfdldmlgsdalddfdldmlgsdalddfdldmlgslpsasvefegsggpsgqis
nqalalapssapvlaqtmvpssamvplaqppapapvltpgppgslsapvpkstqagegtlseallhlq
fdadedlgallgnstdpgvftdlasvdnsefaqllnqgvsmshstaepmlmeypeaitrlvtgsqrpp
dpaptplgtsglpnglsgdedfssiadmdfsallsqisssgqggggsgfsvdtsalldlfspsvtvpd
mslpdldsslasiqellspqepprppeaensspdsgkqlvhytaqplflldpgsvdtgsndlpvlfel
gegsvfsegdgfaedptislitgseppkakdptvs*
DNA sequence fordCas9-VPH (in backbone pNH 23): pNI137;
lowercase underlined = VPH;
capital underlined = dCas9.
SEQ ID NO: 73
atggactacaaagaccatgacggtgattataaagatcatgacatcgattacaaggatgacgatgacaa
gcacgttaaccccaagaagaagaggaaggtgggccgcggaATGGACAAGAAGTACTCCATTGGGCTCG
CCATCGGCACAAACAGCGTCGGCTGGGCCGTCATTACGGACGAGTACAAGGTGCCGAGCAAAAAATTC
AAAGTTCTGGGCAATACCGATCGCCACAGCATAAAGAAGAACCTCATTGGCGCCCTCCTGTTCGACTC
CGGGGAAACCGCCGAAGCCACGCGGCTCAAAAGAACAGCACGGCGCAGATATACCCGCAGAAAGAATC
GGATCTGCTACCTGCAGGAGATCTTTAGTAATGAGATGGCTAAGGTGGATGACTCTTTCTTCCATAGG
CTGGAGGAGTCCTTTTTGGTGGAGGAGGATAAAAAGCACGAGCGCCACCCAATCTTTGGCAATATCGT
GGACGAGGTGGCGTACCATGAAAAGTACCCAACCATATATCATCTGAGGAAGAAGCTTGTAGACAGTA
CTGATAAGGCTGACTTGCGGTTGATCTATCTCGCGCTGGCGCATATGATCAAATTTCGGGGACACTTC
CTCATCGAGGGGGACCTGAACCCAGACAACAGCGATGTCGACAAACTCTTTATCCAACTGGTTCAGAC
TTACAATCAGCTTTTCGAAGAGAACCCGATCAACGCATCCGGAGTTGACGCCAAAGCAATCCTGAGCG
CTAGGCTGTCCAAATCCCGGCGGCTCGAAAACCTCATCGCACAGCTCCCTGGGGAGAAGAAGAACGGC
CTGTTTGGTAATCTTATCGCCCTGTCACTCGGGCTGACCCCCAACTTTAAATCTAACTTCGACCTGGC
CGAAGATGCCAAGCTTCAACTGAGCAAAGACACCTACGATGATGATCTCGACAATCTGCTGGCCCAGA
TCGGCGACCAGTACGCAGACCTTTTTTTGGCGGCAAAGAACCTGTCAGACGCCATTCTGCTGAGTGAT
ATTCTGCGAGTGAACACGGAGATCACCAAAGCTCCGCTGAGCGCTAGTATGATCAAGCGCTATGATGA
GCACCACCAAGACTTGACTTTGCTGAAGGCCCTTGTCAGACAGCAACTGCCTGAGAAGTACAAGGAAA
TTTTCTTCGATCAGTCTAAAAATGGCTACGCCGGATACATTGACGGCGGAGCAAGCCAGGAGGAATTT
TACAAATTTATTAAGCCCATCTTGGAAAAAATGGACGGCACCGAGGAGCTGCTGGTAAAGCTTAACAG
AGAAGATCTGTTGCGCAAACAGCGCACTTTCGACAATGGAAGCATCCCCCACCAGATTCACCTGGGCG
AACTGCACGCTATCCTCAGGCGGCAAGAGGATTTCTACCCCTTTTTGAAAGATAACAGGGAAAAGATT
GAGAAAATCCTCACATTTCGGATACCCTACTATGTAGGCCCCCTCGCCCGGGGAAATTCCAGATTCGC
GTGGATGACTCGCAAATCAGAAGAGACCATCACTCCCTGGAACTTCGAGGAAGTCGTGGATAAGGGGG
CCTCTGCCCAGTCCTTCATCGAAAGGATGACTAACTTTGATAAAAATCTGCCTAACGAAAAGGTGCTT
CCTAAACACTCTCTGCTGTACGAGTACTTCACAGTTTATAACGAGCTCACCAAGGTCAAATACGTCAC
AGAAGGGATGAGAAAGCCAGCATTCCTGTCTGGAGAGCAGAAGAAAGCTATCGTGGACCTCCTCTTCA
AGACGAACCGGAAAGTTACCGTGAAACAGCTCAAAGAAGACTATTTCAAAAAGATTGAATGTTTCGAC
TCTGTTGAAATCAGCGGAGTGGAGGATCGCTTCAACGCATCCCTGGGAACGTATCACGATCTCCTGAA
AATCATTAAAGACAAGGACTTCCTGGACAATGAGGAGAACGAGGACATTCTTGAGGACATTGTCCTCA
CCCTTACGTTGTTTGAAGATAGGGAGATGATTGAAGAACGCTTGAAAACTTACGCTCATCTCTTCGAC
GACAAAGTCATGAAACAGCTCAAGAGGCGCCGATATACAGGATGGGGGCGGCTGTCAAGAAAACTGAT
CAATGGGATCCGAGACAAGCAGAGTGGAAAGACAATCCTGGATTTTCTTAAGTCCGATGGATTTGCCA
ACCGGAACTTCATGCAGTTGATCCATGATGACTCTCTCACCTTTAAGGAGGACATCCAGAAAGCACAA
GTTTCTGGCCAGGGGGACAGTCTTCACGAGCACATCGCTAATCTTGCAGGTAGCCCAGCTATCAAAAA
GGGAATACTGCAGACCGTTAAGGTCGTGGATGAACTCGTCAAAGTAATGGGAAGGCATAAGCCCGAGA
ATATCGTTATCGAGATGGCCCGAGAGAACCAAACTACCCAGAAGGGACAGAAGAACAGTAGGGAAAGG
ATGAAGAGGATTGAAGAGGGTATAAAAGAACTGGGGTCCCAAATCCTTAAGGAACACCCAGTTGAAAA
CACCCAGCTTCAGAATGAGAAGCTCTACCTGTACTACCTGCAGAACGGCAGGGACATGTACGTGGATC
AGGAACTGGACATCAATCGGCTCTCCGACTACGACGTGGATGCCATCGTGCCCCAGTCTTTTCTCAAA
GATGATTCTATTGATAATAAAGTGTTGACAAGATCCGATAAAAATAGAGGGAAGAGTGATAACGTCCC
CTCAGAAGAAGTTGTCAAGAAAATGAAAAATTATTGGCGGCAGCTGCTGAACGCCAAACTGATCACAC
AACGGAAGTTCGATAATCTGACTAAGGCTGAACGAGGTGGCCTGTCTGAGTTGGATAAAGCCGGCTTC
ATCAAAAGGCAGCTTGTTGAGACACGCCAGATCACCAAGCACGTGGCCCAAATTCTCGATTCACGCAT
GAACACCAAGTACGATGAAAATGACAAACTGATTCGAGAGGTGAAAGTTATTACTCTGAAGTCTAAGC
TGGTCTCAGATTTCAGAAAGGACTTTCAGTTTTATAAGGTGAGAGAGATCAACAATTACCACCATGCG
CATGATGCCTACCTGAATGCAGTGGTAGGCACTGCACTTATCAAAAAATATCCCAAGCTTGAATCTGA
ATTTGTTTACGGAGACTATAAAGTGTACGATGTTAGGAAAATGATCGCAAAGTCTGAGCAGGAAATAG
GCAAGGCCACCGCTAAGTACTTCTTTTACAGCAATATTATGAATTTTTTCAAGACCGAGATTACACTG
GCCAATGGAGAGATTCGGAAGCGACCACTTATCGAAACAAACGGAGAAACAGGAGAAATCGTGTGGGA
CAAGGGTAGGGATTTCGCGACAGTCCGGAAGGTCCTGTCCATGCCGCAGGTGAACATCGTTAAAAAGA
CCGAAGTACAGACCGGAGGCTTCTCCAAGGAAAGTATCCTCCCGAAAAGGAACAGCGACAAGCTGATC
GCACGCAAAAAAGATTGGGACCCCAAGAAATACGGCGGATTCGATTCTCCTACAGTCGCTTACAGTGT
ACTGGTTGTGGCCAAAGTGGAGAAAGGGAAGTCTAAAAAACTCAAAAGCGTCAAGGAACTGCTGGGCA
TCACAATCATGGAGCGATCAAGCTTCGAAAAAAACCCCATCGACTTTCTCGAGGCGAAAGGATATAAA
GAGGTCAAAAAAGACCTCATCATTAAGCTTCCCAAGTACTCTCTCTTTGAGCTTGAAAACGGCCGGAA
ACGAATGCTCGCTAGTGCGGGCGAGCTGCAGAAAGGTAACGAGCTGGCACTGCCCTCTAAATACGTTA
ATTTCTTGTATCTGGCCAGCCACTATGAAAAGCTCAAAGGGTCTCCCGAAGATAATGAGCAGAAGCAG
CTGTTCGTGGAACAACACAAACACTACCTTGATGAGATCATCGAGCAAATAAGCGAATTCTCCAAAAG
AGTGATCCTCGCCGACGCTAACCTCGATAAGGTGCTTTCTGCTTACAATAAGCACAGGGATAAGCCCA
TCAGGGAGCAGGCAGAAAACATTATCCACTTGTTTACTCTGACCAACTTGGGCGCGCCTGCAGCCTTC
AAGTACTTCGACACCACCATAGACAGAAAGCGGTACACCTCTACAAAGGAGGTCCTGGACGCCACACT
GATTCATCAGTCAATTACGGGGCTCTATGAAACAAGAATCGACCTCTCTCAGCTCGGTGGAGACagca
gggctgaccccaagaagaagaggaaggtggctagcgatgctttagacgattttgacttagatatgctt
ggttcagacgcgttagacgacttcgacctagacatgttaggctcagatgcattggacgacttcgattt
agatatgttgggctccgatgccctagatgactttgatetagatatgctagggtcactacccagcgcca
gcgtcgagttcgaaggcagcggcgggccttcagggcagatcagcaaccaggccctggctctggcccct
agctccgctccagtgctggcccagactatggtgccctctagtgctatggtgcctctggcccagccacc
tgctccagcccctatgctgaccccaggaccaccccagtcactgagcgccccagtgcccaagtctacac
aggccggcgaggggactctgagtgaagctctgctgcacctgcagttcgacgctgatgaggacctggga
gctctgctggggaacagcaccgatcccggagtgttcacagatctggcctccgtggacaactctgagtt
tcagcagctgctgaatcagggcgtgtccatgtctcatagtacagccgaaccaatgctgatggagtacc
ccgaagccattacccggctggtgaccggcagccagcggccccccgaccccgctccaactcccctggga
accagcggcctgcctaatgggctgtccggagatgaagacttctcaagcatcgctgatatggactttag
tgccctgctgtcacagatttcctctagtgggcagggaggaggtggaagcggcttcagcgtggacacca
gtgccctgctggacctgttcagcccctcggtgaccgtgcccgacatgagcctgcctgaccttgacagc
agcctggccagtatccaagagctcctgtctccccaggagccccccaggcctcccgaggcagagaacag
cagcccggattcagggaagcagctggtgcactacacagcgcagccgctgttcctgctggaccccggct
ccgtggacaccgggagcaacgacctgccggtgctgtttgagctgggagagggctcctacttctccgaa
ggggacggcttcgccgaggaccccaccatctccctgctgacaggctcggagcctcccaaagccaagga
ccccactgtctcc
Amino acid sequence fordCas9-VPH
(corresponding to SEQ ID NO: 73); lowercase
underlined = VPH; capital underlined = dCas9.
SEQ ID NO: 74
dykdhdgaykdhdidykaddddkhvnpkkkrkvgrgMDKKYSIGLAIGTNSVGWAVTDEYKVPSKKFK
VLGNTDRHSIKKNLIGALLFDSGETAEATRLKRTARRRYTRRKNRICYLQEIFSNEMAKVDDSFFHRL
EESFLVEEDKKHERHPIFGNIVDEVAYHEKYPTTYHLRKKLVDSTDKADLRLIYLALAHMIKFRGHFL
IEGDLNPDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAHLFDD
KVMKQLKRRRYTGWGRLSRKLINGIRDKQSGKTILDFLKSDGFANRNEMQLIHDDSLTFKEDIQKAQV
SGQGDSLHEHIANLAGSPAIKKGILQTVKVVDELVKVMGRHKPENTVIEMARENQTTQKGQKNSRERM
KRIEEGIKELGSQILKEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGDsradpkkkrkvasdalddfdldmlq
sdalddfdldmlqsdalddfdldmlqsdalddfdldmlgslpsasvefegsggpsgqisnqalalaps
sapvlagtmvpssamvplaqppapapvltpgppqslsapvpkstqagegtlseallhlqfdadedlga
llgnstdpgvftdlasvdnsefqqllnggvsmshstaepmlmeypeaitrlvtgsqrppdpaptplgt
sglpnglsgdedfssiadmdfsallsqisssgqggggsgfsvdtsalldlfspsvtvpdmslpdldss
lasiqellspgepprppeaensspdsqkqlvhytaqplflldpgsvdtgsndlpvlfelgeqsyfseg
dgfaedptislltqseppkakdptvs
DNA sequence for VPH-dCas9-VPH (in backbone pNI36): pNH 15; lowercase
underlined = VPH; capital underlined = dCas9.
SEQ ID NO: 75
atggatgctttagacgattttgacttagatatgcttggttcagacgcgttagacgacttcgacctaga
catgttaggctcagatgcattggacgacttcgatttagatatgttgggctccgatgccctagatgact
ttgatctagatatgctagggtcactacccagcgccagcgtcgagttcgaaggcagcggcgggccttca
gggcagatcagcaaccaggccctggctctggcccctagctccgctccagtgctggcccagactatggt
gccctctagtgctatggtgcctctggcccagccacctgctccagcccctgtgctgaccccaggaccac
cccagtcactgagcgccccagtgcccaagtctacacaggccggcgaggggactctgagtgaagctctg
ctgcacctgcagttcgacgctgatgaggacctgggagctctgctggggaacagcaccgatcccggagt
gttcacagatctggcctccgtggacaactctgagtttcagcagctgctgaatcagggcgtgtccatgt
ctcatagtacagccgaaccaatgctgatggagtaccccgaagccattacccggctggtgaccggcagc
cagcggccccccgaccccgctccaactcccctgggaaccagcggcctgcctaatgggctgtccggaga
tgaagacttctcaagcatcgctgatatggactttagtgccctgctgtcacagatttcctctagtgggc
agggaggaggtggaagcggcttcagcgtggacaccagtgccctgctggacctgttcagcccctcggtg
accgtgcccgacatgagcctgcctgaccttgacagcagcctggccagtatccaagagctcctgtctcc
ccaggagccccccaggcctcccgaggcagagaacagcagcccggattcagggaagcagctggtgcact
acacagcgcagccgctgttcctgctggaccccggctccgtggacaccgggagcascgacctgccggtg
ctgtttgagctgggagagggctcctacttctccgaaggggacggcttcgccgaggaccccaccatctc
cctgctgacaggctcggagcctcccaaagccaaggaccccactgtctccggctctggaggatctggcg
gctctagcgccaccATGGACAAGAAGTACAGCATCGGCCTGGCCATCGGCACCAACTCTGTGGGCTGG
GCCGTGATCACCGACGAGTACAAGGTGCCCAGCAAGAAATTCAAGGTGCTGGGCAACACCGACCGGCA
CAGCATCAAGAAGAACCTGATCGGAGCCCTGCTGTTCGACAGCGGCGAAACAGCCGAGGCCACCCGGC
TGAAGAGAACCGCCAGAAGAAGATACACCAGACGGAAGAACCGGATCTGCTATCTGCAAGAGATCTTC
AGCAACGAGATGGCCAAGGTGGACGACAGCTTCTTCCACAGACTGGAAGAGTCCTTCCTGGTGGAAGA
GGATAAGAAGCACGAGCGGCACCCCATCTTCGGCAACATCGTGGACGAGGTGGCCTACCACGAGAAGT
ACCCCACCATCTACCACCTGAGAAAGAAACTGGTGGACAGCACCGACAAGGCCGACCTGCGGCTGATC
TATCTGGCCCTGGCCCACATGATCAAGTTCCGGGGCCACTTCCTGATCGAGGGCGACCTGAACCCCGA
CAACAGCGACGTGGACAAGCTGTTCATCCAGCTGGTGCAGACCTACAACCAGCTGTTCGAGGAAAACC
CCATCAACGCCAGCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCTGATTGCCCTGAG
CCTGGGCCTGACCCCCAACTTCAAGAGCAACTTCGACCTGGCCGAGGATGCCAAACTGCAGCTGAGCA
AGGACACCTACGACGACGACCTGGACAACCTGCTGGCCCAGATCGGCGACCAGTACGCCGACCTGTTT
CTGGCCGCCAAGAACCTGTCCGACGCCATCCTGCTGAGCGACATCCTGAGAGTGAACACCGAGATCAC
CAAGGCCCCCCTGAGCGCCTGTATGATCAAGAGATACGACGAGCAGGAGGAGGACCTGACCCTGCTGA
AAGCTCTCGTGCGGCAGCAGCTGCCTGAGAAGTACAAAGAGATTTTCTTCGACCAGAGCAAGAACGGC
TACGCCGGCTACATTGACGGCGGAGCCAGCCAGGAAGAGTTCTACAAGTTCATCAAGCCCATCCTGGA
AAAGATGGACGGCACCGAGGAACTGCTCGTGAAGCTGAACAGAGAGGACCTGCTGCGGAAGCAGCGGA
CCTTCGACAACGGCAGCATCCCCCACCAGATCCACCTGGGAGAGCTGCACGCCATTCTGCGGCGGCAG
GAAGATTTTTACCCATTCCTGAAGGACAACCGGGAAAAGATCGAGAAGATCCTGACCTTCCGCATCCC
CTACTACGTGGGCCCTCTGGCCAGGGGAAACAGCAGATTCGCCTGGATGACCAGAAAGAGCGAGGAAA
CCATCACCCCCTGGAACTTCGAGGAAGTGGTGGACAAGGGCGCTTCCGCCCAGAGCTTCATCGAGCGG
ATGACCAACTTCGATAAGAACCTGCCCAACGAGAAGGTGCTGCCCAAGCACAGCCTGCTGTACGAGTA
CTTCACCGTGTATAACGAGCTGACCAAAGTGAAATACGTGACCGAGGGAATGAGAAAGCCCGCCTTCC
TGAGCGGCGAGCAGAAAAAGGCCATCGTGGACCTGCTGTTCAAGACCAACCGGAAAGTGACCGTGAAG
CAGCTGAAAGAGGACTACTTCAAGAAAATCGAGTGCTTCGACTCCGTGGAAATCTCCGGCGTGGAAGA
TCGGTTCAACGCCTCCCTGGGCACATACCACGATCTGCTGAAAATTATCAAGGACAAGGACTTCCTGG
ACAATGAGGAAAACGAGGACATTCTGGAAGATATCGTGCTGACCCTGACACTGTTTGAGGACAGAGAG
ATGATCGAGGAACGGCTGAAAACCTATGCCCACCTGTTCGACGACAAAGTGATGAAGCAGCTGAAGCG
GCGGAGATACACCGGCTGGGGCAGGCTGAGCCGGAAGCTGATCAACGGCATCCGGGACAAGCAGTCCG
GCAAGACAATCCTGGATTTCCTGAAGTCCGACGGCTTCGCCAACAGAAACTTCATGCAGCTGATCCAC
GACGACAGCCTGACCTTTAAAGAGGACATCCAGAAAGCCCAGGTGTCCGGCCAGGGCGATAGCCTGCA
CGAGCACATTGCCAATCTGGCCGGCAGCCCCGCCATTAAGAAGGGCATCCTGCAGACAGTGAAGGTGG
TGGACGAGCTCGTGAAAGTGATGGGCCGGCACAAGCCCGAGAACATCGTGATCGAAATGGCCAGAGAG
AACCAGACCACCCAGAAGGGACAGAAGAACAGCCGCGAGAGAATGAAGCGGATCGAAGAGGGCATCAA
AGAGCTGGGCAGCCAGATCCTGAAAGAACACCCCGTGGAAAACACCCAGCTGCAGAACGAGAAGCTGT
ACCTGTACTACCTGCAGAATGGGCGGGATATGTACGTGGACCAGGAACTGGACATCAACCGGCTGTCC
GACTACGATGTGGACGCCATCGTGCCTCAGAGCTTTCTGAAGGACGACTCCATCGACAACAAGGTGCT
GACCAGAAGCGACAAGAACCGGGGCAAGAGCGACAACGTGCCCTCCGAAGAGGTCGTGAAGAAGATGA
AGAACTACTGGCGGCAGCTGCTGAACGCCAAGCTGATTACCCAGAGAAAGTTCGACAATCTGACCAAG
GCCGAGAGAGGCGGCCTGAGCGAACTGGATAAGGCCGGCTTCATCAAGAGACAGCTGGTGGAAACCCG
GCAGATCACAAAGCACGTGGCACAGATCCTGGACTCCCGGATGAACACTAAGTACGACGAGAATGACA
AGCTGATCCGGGAAGTGAAAGTGATCACCCTGAAGTCCAAGCTGGTGTCCGATTTCCGGAAGGATTTC
CAGTTTTACAAAGTGCGCGAGATCAACAATACCACCACGCCCACGACGCCTACCTAGAACGCCGTCGT
GGGAACCGCCCTGATCAAAAAGTACCCTAAGCTGGAAAGCGAGTTCGTGTACGGCGACTACAAGGTGT
ACGACGTGCGGAAGATGATCGCCAAGAGCGAGCAGGAAATCGGCAAGGCTACCGCCAAGTACTTCTTC
TACAGCAACATCATGAACTTTTTCAAGACCGAGATTACCCTGGCCAACGGCGAGATCCGGAAGCGGCC
TCTGATCGAGACAAACGGCGAAACCGGGGAGATCGTGTGGGATAAGGGCCGGGATTTTGCCACCGTGC
GGAAAGTGCTGAGCATGCCCCAAGTGAATATCGTGAAAAAGACCGAGGTGCAGACAGGCGGCTTCAGC
AAAGAGTCTATCCTGCCCAAGAGGAACAGCGATAAGCTGATCGCCAGAAAGAAGGACTGGGACCCTAA
GAAGTACGGCGGCTTCGAAAGCCCCACCGTGGCCTATTCTGTGCTGGTGGTGGCCAAAGTGGAAAAGG
GCAAGTCCAAGAAACTGAAGAGTGTGAAAGAGCTGCTGGGGATCACCATCATGGAAAGAAGCAGCTTC
GAGAAGAATCCCATCGACTTTCTGGAAGCCAAGGGCTACAAAGAAGTGAAAAAGGACCTGATCATCAA
GCTGCCTAAGTACTCCCTGTTCGAGCTGGAAAACGGCCGGAAGAGAATGCTGGCCTCTGCCGGCGAAC
TGCAGAAGGGAAACGAACTGGCCCTGCCCTCCAAATATGTGAACTTCCTGTACCTGGCCAGCCACTAT
GAGAAGCTGAAGGGCTCCCCCGAGGATAATGAGCAGAAACAGCTGTTTGTGGAACAGCACAAGCACTA
CCTGGACGAGATCATCGAGCAGATCAGCGAGTTCTCCAAGAGAGTGATCCTGGCCGACGCTAATCTGG
ACAAAGTGCTGTCCGCCTACAACAAGCACCGGGATAAGCCCATCAGAGAGCAGGCCGAGAATATCATC
CACCTGTTTACCCTGACCAATCTGGGAGCCCCTGCCGCCTTCAAGTACTTTGACACCACCATCGACCG
GAAGAGGTACACCAGCACCAAAGAGGTGCTGGACGCCACCCTGATCCACCAGAGCATCACCGGCCTGT
ACGAGACACGGATCGACCTGTCTCAGCTGGGAGGCGACaagcgacctgccgccacaaagaaggctgga
caggctaagaagaagaaactggactctggaggatccgactacaaagaccatgacggtgattataaaga
tcatgacatcgattacaaggatgacgatgacaagggaggatccaaggagaagagtgcttgtcctaaag
atccagccaaacctccggccaaggcacaagttgtgggatggccaccggtgagatcataccggaagaac
gtgatggtttcctgccaaaaatcaagcggtggcccggaggcggcggcgttcgtgaaggtatcaatgga
cggagcaccgtacttgaggaaaatcgatttgaggatgtataaaggcggatctggcggctctggaggat
ccagcgatgctttagacgattttgacttagatatgcttggttcagacgcgttagacgacttcgaccta
gacatgttaggctcagatgcattggacgacttcgatttagatatgttgggctccgatgccctagatga
ctttgatctagatatgctagggtcactacccagcgctagcgtcgagttcgaaggcagcggcgggcctt
cagggcagatcagcaaccaggccctggctctggcccctagctccgctccagtgctggcccagactatg
gtgccctctagtgctatggtgcctctggcccagccacctgctccagcccctgtgctgaccccaggacc
accccagtcactgagcgctccagtgcccaagtctacacaggccggcgaggggactctgagtgaagctc
tgctgcacctgcagttcgacgctgatgaggacctgggagctctgctggggaacagcaccgatcccgga
gtgttcacagatctggcctccgtggacaactctgagtttcagcagctgctgaatcagggcgtgtccat
gtctcatagtacagccgaaccaatgctgatggagtaccccgaagccattacccggctggtgaccggca
gccagcggccccccgaccccgctccaactcccctgggaaccagcggcctgcctaatgggctgtccgga
gatgaagacttctcaagcatogctgatatggactttagtgccctgctgtcacagatttcctctagtgg
gcagggaggaggtggaagcggcttcagcgtggacaccagtgccctgctggacctgttcagcccctcgg
tgaccgtgcccgacatgagcctgcctgaccttgacagcagcctggccagtatccaagagctcctgtct
ccccaggagccccccaggcctcccgaggcagagaacagcagcccggattcagggaagcagctggtgca
ctacacagcgcagccgctgttcctgctggaccccggctccgtggacaccgggagcaacgacctgccgg
tgctgtttgagctgggagagggctcctacttctccgaaggggacggcttcgccgaggaccccaccatc
tccctgctgacaggctcggagcctcccaaagccaaggaccccactgtctcctga
Amino acid sequence for VPH-dCas9-VPH
(corresponding to SEQ ID NO: 75); lowercase
underlined = VPH; capital underlined = dCas9.
SEQ ID NO: 76
dalddfdldmlgsdalddfdldmlgsdalddfdldmlgsdalddfaldmlgslpsasvefegsggpsg
qisnqalalapssapvlaqtmvpssamvplaqppapapvltpgppqslsapvpkstqagegtlseall
hlqfdadedlgallgnstdpgvftdlasvdnsefqqllnqgvsmshstaepmlmeypeaitrlvtgsq
rppdpaptplgtsqlpnglsqdedfssiadmdfsallsqisssgqggqqsgfsvdtsalldlfspsvt
vpdmslpdldsslasiqellspqepprppeaensspdsgkqlvhytaqplflldpgsvdtgsndlpvl
felgegsyfsegdqfaedptislltgseppkakdptvsgsggsggssatMDKKYSIGLAIGTNSVGWA
VITDEYKVPSKKFKVLGNTDRHSIKKNLIGALLFDSGETAEATRLKRTARRRYTRRKNRICYLQEIFS
NEMAKVDDSFFHRLEESFLVEEDKKHERHPIFGNIVDEVAYHEKYPTIYHLRKKLVDSTDKADLRLIY
LAIAHMIKFRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIGDQYADLFL
AAKNLSDAILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRKVTVKQ
LKEDYFKKIECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGLLQTVKVVDELVKVMGRHKPENIVIEMAREN
QTTQKGQKNSRERMKRIEEGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKSKLVSDERKDEQ
FYKVREINNYHHAHDAYLNAVVGTALIKKYPKLESEFVYGDYKVYDVRRMIAKSEQEIGKATAKYFFY
SNIMNFFKTEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGITIMERSSFE
KNPIDFLEAKGYKEVKKDLIIKLPKYSLFELENGRKRMLASAGELQKGNELALPSKYVNFLYLASHYE
KLKGSPEDNEQKQLFVEQHKHYLDEIIEQISEFSKRVTLADANLDKVLSAYNKHRDKPIREQAENIIH
LFTLTNLGAPAAFKYFDTTIDRKRYTSTKEVLDATLIHQSITGLYETRIDLSQLGGDkrpaatkkagq
akkkkldsggsdykdhdgdykdhdidykddddkggskeksacpkdpakppakaqvvgwppvrsyrknv
mvscqkssqqpeaaafvkvsmdgapylrkidirmykgqsagsggssdalddfdldmlgsdalddfdid
mlgsdalddfdldmlgsdalddfdldmlgslpsasvefegsggpsgqisnqalalapssapvlaqtmv
pssamvplaqppapapvltpgppgslsapypkstgagegtlseallhlqfdadedlgallgnstdpqv
ftdlasvansefqqllnqgvsmshstaepmlmeypeaitrivtgsqrppdpaptplgtsqlpnglsqd
edfssiadmdfsailsqisssgqggggsgfsvdtsalldlfspsvtvpdmslpdldsslasiqellsp
qepprppeaensspdsgkqlvhytaqplflldpgsvdtgsndlpvlfelgegsyfsegdqfaedptis
lllqseppkakdptvs*
DNA sequence fordCas9-VPR (in backbone pNI3S):
pNi47; lowercase underlined = VPR;
capital underlined = dCas9.
SEQ ID NO: 77
ATGGACAAGAAGTACAGCATCGGCCTGGCCATCGGCACCAACTCTGTGGGCTGGGCCGTGATCACCGA
CGAGTACAAGGTGCCCAGCAAGAAATTCAAGGTGCTGGGCAACACCGACCGGCACAGCATCAAGAAGA
ACCTGATCGGAGCCCTGCTGTTCGACAGCGGCGAAACAGCCGAGGCCACCCGGCTGAAGAGAACCGCC
AGAAGAAGATACACCAGACGGAAGAACCGGATCTGCTATCTGCAAGAGATCTTCAGCAACGAGATGGC
CAAGGTGGACGACAGCTTCTTCCACAGACTGGAAGAGTCCTTCCTGGTGGAAGAGGATAAGAAGCACG
AGCGGCACCCCATCTTCGGCAACATCGTGGACGAGGTGGCCTACCACGAGAAGTACCCCACCATCTAC
CACCTGAGAAAGAAACTGGTGGACAGCACCGACAAGGCCGACCTGCGGCTGATCTATCTGGCCCTGGC
CCACATGATCAAGTTCCGGGGCCACTTCCTGATCGAGGGCGACCTGAACCCCGACAACAGCGACGTGG
ACAAGCTGTTCATCCAGCTGGTGCAGACCTACAACCAGCTGTTCGAGGAAAACCCCATCAACGCCAGC
GGCGTGGACGCCAAGGCCATCCTGTCTGCCAGACTGAGCAAGAGCAGACGGCTGGAAAATCTGATCGC
CCAGCTGCCCGGCGAGAAGAAGAATGGCCTGTTCGGAAACCTGATTGCCCTGAGCCTGGGCCTGACCC
CCAACTTCAAGAGCAACTTCGACCTGGCCGAGGATGCCAAACTGCAGCTGAGCAAGGACACCTACGAC
GACGACCTGGACAACCTGCTGGCCCAGATCGGCGACCAGTACGCCGACCTGTTTCTGGCCGCCAAGAA
CCTGTCCGACGCCATCCTGCTGAGCGACATCCTGAGAGTGAACACCGAGATCACCAAGGCCCCCCTGA
GCGCCTCTATGATCAAGAGATACGACGAGCACCACCAGGACCTGACCCTGCTGAAAGCTCTCGTGCGG
CAGCAGCTGCCTGAGAAGTACAAAGAGATTTTCTTCGACCAGAGCAAGAACGGCTACGCCGGCTACAT
TGACGGCGGAGCCAGCCAGGAAGAGTTCTACAAGTTCATCAAGCCCATCCTGGAAAAGATGGACGGCA
CCGAGGAACTGCTCGTGAAGCTGAACAGAGAGGACCTGCTGCGGAAGCAGCGGACCTTCGACAACGGC
AGCATCCCCCACCAGATCCACCTGGGAGAGCTGCACGCCATTCTGCGGCGGCAGGAAGATTTTTACCC
ATTCCTGAAGGACAACCGGGAAAAGATCGAGAAGATCCTGACCTTCCGCATCCCCTACTACGTGGGCC
CTCTGGCCAGGGGAAACAGCAGATTCGCCTGGATGACCAGAAAGAGCGAGGAAACCATCACCCCCTGG
AACTTCGAGGAAGTGGTGGACAAGGGCGCTTCCGCCCAGAGCTTCATCGAGCGGATGACCAACTTCGA
TAAGAACCTGCCCAACGAGAAGGTGCTGCCCAAGCACAGCCTGCTGTACGAGTACTTCACCGTGTATA
ACGAGCTGACCAAAGTGAAATACGTGACCGAGGGAATGAGAAAGCCCGCCTTCCTGAGCGGCGAGCAG
AAAAAGGCCATCGTGGACCTGCTGTTCAAGACCAACCGGAAAGTGACCGTGAAGCAGCTGAAAGAGGA
CTACTTCAAGAAAATCGAGTGCTTCGACTCCGTGGAAATCTCCGGCGTGGAAGATCGGTTCAACGCCT
CCCTGGGCACATACCACGATCTGCTGAAAATTATCAAGGACAAGGACTTCCTGGACAATGAGGAAAAC
GAGGACATTCTGGAAGATATCGTGCTGACCCTGACACTGTTTGAGGACAGAGAGATGATCGAGGAACG
GCTGAAAACCTATGCCCACCTGTTCGACGACAAAGTGATGAAGCAGCTGAAGCGGCGGAGATACACCG
GCTGGGGCAGGCTGAGCCGGAAGCTGATCAACGGCATCCGGGACAAGCAGTCCGGCAAGACAATCCTG
GATTTCCTGAAGTCCGACGGCTTCGCCAACAGAAACTTCATACAGCTGATCCACGACGACAGCCTGAC
CTTTAAAGAGGACATCCAGAAAGCCCAGGTGTCCGGCCAGGGCGATAGCCTGCACGAGCACATTGCCA
ATCTGGCCGGCAGCCCCGCCATTAAGAAGGGCATCCTGCAGACAGTGAAGGTGGTGGACGAGCTCGTG
AAAGTGATGGGCCGGCACAAGCCCGAGAACATCGTGATCGAAATGGCCAGAGAGAACCAGACCACCCA
GAAGGGACAGAAGAACAGCCGCGAGAGAATGAAGCGGATCGAAGAGGGCATCAAAGAGCTGGGCAGCC
AGATCCTGAAAGAACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGACTACGATGTGGA
CGCCATCGTGCCTCAGAGCTTTCTGAAGGACGACTCCATCGACAACAAGGTGCTGACCAGAAGCGACA
AGAACCGGGGCAAGAGCGACAACGTGCCCTCCGAAGAGGTCGTGAAGAAGATGAAGAACTACTGGCGG
CAGCTGCTGAACGCCAAGCTGATTACCCAGAGAAAGTTCGACAATCTGACCAAGGCCGAGAGAGGCGG
CCTGAGCGAACTGGATAAGGCCGGCTTCATCAAGAGACAGCTGGTGGAAACCCGGCAGATCACAAAGC
ACGTGGCACAGATCCTGGACTCCCGGATGAACACTAAGTACGACGAGAATGACAAGCTGATCCGGGAA
GTGAAAGTGATCACCCTGAAGTCCAAGCTGGTGTCCGATTTCCGGAAGGATTTCCAGTTTTACAAAGT
GCGCGAGATCGGCAACTACCACCACGCCCACGACGCCTACCTGAACGCCGTCGTGGGAACCGCCCTGA
TCAAAAAGTACCCTAAGCTGGAAAGCGAGTTCGTGTACGGCGACTACAAGGTGTACGACGTGCGGAAG
ATGATCGCCAAGAGCGAGCAGGAAATCGGCAAGGCTACCGCCAAGTACTTCTTCTACAGCAACATCAT
GAACTTTTTCAAGACCAGATTACCCTGGCCAACGGCGAGAGTCCGGAAGCGGCCTCTGATCGAGACAA
ACGGCGAAACCGGGGAGATCGTGTGGGATAAGGGCCGGGATTTTGCCACCGTGCGGAAAGTGCTGAGC
ATGCCCCAAGTGAATATCGTGAAAAAGACCGAGGTGCAGACAGGCGGCTTCAGCAAAGAGTCTATCCT
GCCCAAGAGGAACAGCGATAAGCTGATCGCCAGAAAGAAGGACTGGGACCCTAAGAAGTACGGCGGCT
TCGACAGCCCCACCGTGGCCTATTCTGTGCTGGTGGTGGCCAAAGTGGAAAAGGGCAAGTCCAAGAAA
CTGAAGAGTGTGAAAGAGCTGCTGGGGATCACCATCATGGAAAGAAGCAGCTTCGAGAAGAATCCCAT
CGACTTTCTGGAAGCCAAGGGCTACAAAGAAGTGAAAAAGGACCTGATCATCAAGCTGCCTAAGTACT
CCCTGTTCGAGCTGGAAAACGGCCGGAAGAGAATGCTGGCCTCTGCCGGCGAACTGCAGAAGGGAAAC
GAACTGGCCCTGCCCTCCAAATATGTGAACTTCCTGTACCTGGCCAGCCACTATGAGAAGCTGAAGGG
CTCCCCCGAGGATAATGAGCAGAAACAGCTGTTTGTGGAACAGCACAAGCACTACCTGGACGAGATCA
TCGAGCAGATCAGCGAGTTCTCCAAGAGAGTGATCCTGGCCGACGCTAATCTGGACAAAGTGCTGTCC
GCCTACAACAAGCACCGGGATAAGCCCATCAGAGAGCAGGCCGAGAATATCATCCACCTGTTTACCCT
GACCAATCTGGGAGCCCCTGCCGCCTTCAAGTACTTTGACACCACCATCGACCGGAAGAGGTACACCA
GCACCAAAGAGGTGCTGGACGCCACCCTGATCCACCAGAGCATCACCGGCCTGTACGAGACACGGATC
GACCTGTCTCAGCTGGGAGGCGACaagcgacctgccgccacaaagaaggctggacaggctaagaagaa
gaaactggactctggaggatccgactacaaagaccatgacggtgattataaagatcatgacatcgatt
acaaggatgacgatgacaagggaggatccaaggagaagagtgcttgtcctaaagatccagccaaacct
ccggccaaggcacaagttgtgggatggccaccggtgagatcataccggaagaacgtgatggtttcctg
ccaaaaatcaagcggtggcccggaggcggcggcgttcgtgaaggtatcaatggacggagcaccgtact
tgaggaaaatcgatttgaggatgtataaaggcggatctggcggctctggaggatccgatgctttagac
gattttgacttagatatgcttggttcagacgcgttagacgacttcgacctagacatgttaggctcaga
tgcattggacgacttcgatttagatatgttgggctccgatgccctagatgactttgatctagatatgc
taggtagtcccaaaaagaagaggaaagtgggatcccagtatctgcccgacacagatgatagacaccga
atcgaagagaaacgcaagcgaacgtatgaaaccttcaaatcgatcatgaagaaatcgcccttctoggg
tccgaccgatcccaggcccccaccgagaaggattgcggtcccgtcccgctcgtcggccagcgtgccga
agcctgcgccgcagccctaccccttcacgtcgagcctgagcacaatcaattatgacgagttcccgacg
atggtgttcccctcgggacaaatctcacaagcctcggcgctcgcaccagcgcctccccaagtccttcc
gcaagcgcctgccccagcgcctgcaccggcaatggtgtccgccctcgcacaggcccctgcgcccgtcc
ccgtgctcgcgcctggaccgccccaggcggtcgctccaccggctccgaagccgacgcaggccggagag
ggaacactctccgaagcacttcttcaactccagtttgatgacgaggatcttggagcactccttggaaa
ctcgacagaccctgcggtgtttaccgacctcgcgtcagtagataactccgaatttcagcagcttttga
accagggtatcccggtcgcgccacatacaacggagcccatgttgatggaataccccgaagcaatcacg
agacttgtgacgggagcgcagcggcctcccgatcccgcacccgcacctttgggggcacctggcctccc
taacggacttttgagcggcgacgaggatttctcctccatcgccgatatggatttctcagccttgctgt
cacagatttccagcggctctggcagcggcagccgggattccagggaagggatgtttttgccgaagcct
gaggccggctccgctattagtgacgtgtttgagggccgcgaggtgtgccagccaaaacgaatccggcc
atttcatcctccaggaagtccatgggccaaccgcccactccccgccagcctcgcaccaacaccaaccg
gtccagtacatgagccagtcgggtcactgaccccggcaccagtccctcagccactggatccagcgccc
gcagtgactcccgaggccagtcacctattggaggatcccgatgaagagacgagccaggctgtcaaagc
ccttcgggagatggccgatactgtgattccccagaaggaagaggctgcaatctgtagccaaatggacc
tttcccatccgcccccaaggggccatctggatgagctgacaaccacacttgagtccatgaccgaggat
ctgaacctggactcacccctgaccccggaattgaacgagattctggataccttcctgaacgacgagtg
cctcttgcatgccatgcatatcagcacaggactgtccatcttcgacacatctctgttttga
Amino acid sequence fordCas9-VPR
(corresponding to SEQ ID NO: 77); lowercase
underlined = VPR; capital underlined = dCas9.
SEQ ID NO: 78
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFTERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDkrpaatkkaqqakkkkldsggsdykdhdgdykdhdidykddddkggskeksacpkdpakp
pakaqvvgwppvrsyrknvmvscqkssggpeaaafvkvsmdgapylrkidlrmykggsggsggsdald
dfdldmlgsdalddfdldmlgsdalddfdldmlgsdalddfdldmlgspkkkrkvgsqylpdtddrhr
ieekrkrtyetfksimkkspfsgptdprppprriavpsrssasypkpapqpypftsslstinydefpt
mvfpsgqisqasalapappqvlpqapapapapamvsalaqapapvpvlapgppqavappapkptqaqe
gtlseallqlqfddedlgallgnstdpavftdlasvdnsefqqllnqgipvaphttepmlmeypeait
rlvtgaqrppdpapaplqapglpngllsgdedfssiadmdfsallsqissgsgsgsrdsregmflpkp
eagsaisdvfegrevcqpkrirpfhppgspwanrplpaslaptptgpvhepvqsllpapvpqpldpap
avtpeashlledpdeetsqavkalremadtvipqkeeaaicgqmdlshppprghldeltttlesmted
lnldspllpelneildtflndecllhamhistglsifdtslf*
DNA sequence for dCas9-p300c (in backbone pN/36):
pNI37; lowercase underlined = p300c;
capital underlined = dCas9,
SEQ ID NO: 79
ATGGACAAGAAGTACAGCATCGGCCTGGCCATCGGCACCAACTCTGTGGGCTGGGCCGTGATCACCGA
CGAGTACAAGGTGCCCAGCAAGAAATTCAAGGTGCTGGGCAACACCGACCGGCACAGCATCAAGAAGA
ACCTGATCGGAGCCCTGCTGTTCGACAGCGGCGAAACAGCCGAGGCCACCCGGCTGAAGAGAACCGCC
AGAAGAAGATACACCAGACGGAAGAACCGGATCTGCTATCTGCAAGAGATCTTCAGCAACGAGATGGC
CAAGGTGGACGACAGCTTCTTCCACAGACTGGAAGAGTCCTTCCTGGTGGAAGAGGATAAGAAGCACG
AGCGGCACCCCATCTTCGGCAACATCGTGGACGAGGTGGCCTACCACGAGAAGTACCCCACCATCTAC
CACCTGAGAAAGAAACTGGTGGACAGCACCGACAAGGCCGACCTGCGGCTGATCTATCTGGCCCTGGC
CCACATGATCAAGTTCCGGGGCCACTTCCTGATCGAGGGCGACCTGAACCCCGACAACAGCGACGTGG
ACAAGCTGTTCATCCAGCTGGTGCAGACCTACAACCAGCTGTTCGAGGAAAACCCCATCAACGCCAGC
GGCGTGGACGCCAAGGCCATCCTGTCTGCCAGACTGAGCAAGAGCAGACGGCTGGAAAATCTGATCGC
CCAGCTGCCCGGCGAGAAGAAGAATGGCCTGTTCGGAAACCTGATTGCCCTGAGCCTGGGCCTGACCC
CCAACTTCAAGAGCAACTTCGACCTGGCCGAGGATGCCAAACTGCAGCTGAGCAAGGACACCTACGAC
GACGACCTGGACAACCTGCTGGCCCAGATCGGCGACCAGTACGCCGACCTGTTTCTGGCCGCCAAGAA
CCTGTCCGACGCCATCCTGCTGAGCGACATCCTGAGAGTGAACACCGAGATCACCAAGGCCCCCCTGA
GCGCCTCTATGATCAAGAGATACGACGAGCACCACCAGGACCTGACCCTGCTGAAAGCTCTCGTGCGG
CAGCAGCTGCCTGAGAAGTACAAAGAGATTTTCTTCGACCAGAGCAAGAACGGCTACGCCGGCTACAT
TGACGGCGGAGCCAGCCAGGAAGAGTTCTACAAGTTCATCAAGCCCATCCTGGAAAAGATGGACGGCA
CCGAGGAACTGCTCGTGAAGCTGAACAGAGAGGACCTGCTGCGGAAGCAGCGGACCTTCGACAACGGC
AGCATCCCCCACCAGATCCACCTGGGAGAGCTGCACGCCATTCTGCGGCGGCAGGAAGATTTTTACCC
ATTCCTGAAGGACAACCGGGAAAAGATCGAGAAGATCCTGACCTTCCGCATCCCCTACTACGTGGGCC
CTCTGGCCAGGGGAAACAGCAGATTCGCCTGGATGACCAGAAAGAGCGAGGAAACCATCACCCCCTGG
AACTTCGAGGAAGTGGTGGACAAGGGCGCTTCCGCCCAGAGCTTCATCGAGCGGATGACCAACTTCGA
TAAGAACCTGCCCAACGAGAAGGTGCTGCCCAAGCACAGCCTGCTGTACGAGTACTTCACCGTGTATA
ACGAGCTGACCAAAGTGAAATACGTGACCGAGGGAATGAGAAAGCCCGCCTTCCTGAGCGGCGAGCAG
AAAAAGGCCATCGTGGACCTGCTGTTCAAGACCAACCGGAAAGTGACCGTGAAGCAGCTGAAAGAGGA
CTACTTCAAGAAAATCGAGTGCTTCGACTCCGTGGAAATCTCCGGCGTGGAAGATCGGTTCAACGCCT
CCCTGGGCACATACCACGATCTGCTGAAAATTATCAAGGACAAGGACTTCCTGGACAATGAGGAAAAC
GAGGACATTCTGGAAGATATCGTGCTGACCCTGACACTGTTTGAGGACAGAGAGATGATCGAGGAACG
GCTGAAAACCTATGCCCACCTGTTCGACGACAAAGTGATGAAGCAGCTGAAGCGGCGGAGATACACCG
GCTGGGGCAGGCTGAGCCGGAAGCTGATCAACGGCATCCGGGACAAGCAGTCCGGCAAGACAATCCTG
GATTTCCTGAAGTCCGACGGCTTCGCCAACAGAAACTTCATGCAGCTGATCCACGACGACAGCCTGAC
CTTTAAAGAGGACATCCAGAAAGCCCAGGTGTCCGGCCAGGGCGATAGCCTGCACGAGCACATTGCCA
ATCTGGCCGGCAGCCCCGCCATTAAGAAGGGCATCCTGCAGACAGTGAAGGTGGTGGACGAGCTCGTG
AAAGTGATGGGCCGGCACAAGCCCGAGAACATCGTGATCGAAATGGCCAGAGAGAACCAGACCACCCA
GAAGGGACAGAAGAACAGCCGCGAGAGAATGAAGCGGATCGAAGAGGGCATCAAAGAGCTGGGCAGCC
AGATCCTGAAAGAACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGACTACGATGTGGA
CGCCATCGTGCCTCAGAGCTTTCTGAAGGACGACTCCATCGACAACAAGGTGCTGAGGAGAAGCGACA
AGAACCGGGGCAAGAGCGACAACGTGCCCTCCGAAGAGGTCGTGAAGAAGATGAAGAACTACTGGCGG
CAGCTGCTGAACGCCAAGCTGATTACCCAGAGAAAGTTCGACAATCTGACCAAGGCCGAGAGAGGCGG
CCTGAGCGAACTGGATAAGGCCGGCTTCATCAAGAGACAGCTGGTGGAAACCCGGCAGATCACAAAGC
ACGTGGCACAGATCCTGGACTCCCGGATGAACACTAAGTACGACGAGAATGACAAGCTGATCCGGGAA
GTGAAAGTGATCACCCTGAAGTCCAAGCTGGTGTCCGATTTCCGGAAGGATTTCCAGTTTTACAAAGT
GCGCGAGATCAACAACTACCACCACGCCCACGACGCCTACCTGAACGCCGTCGTGGGAACCGCCCTGA
TCAAAAAGTACCCTAAGCTGGAAAGCGAGTTCGTGTACGGCGACTACAAGGTGTACGACGTGCGGAAG
ATGATCGCCAAGAGCGAGCAGGAAATCGGCAAGGCTACCGCCAAGTACTTCTTCTACAGCAACATCAT
GAACTTTTTCAAGACCGAGATTACCCTGGCCAACGGCGAGATCCGGAAGCGGCCTCTGATCGAGACAA
ACGGCGAAACCGGGGAGATCGTGGTGGGATAAGGGCCGGGATTTTTGCCACCGTGCGAAAGTGTGAGC
ATGCCCCAAGTGAATATCGTGAAAAAGACCGAGGTGCAGACAGGCGGCTTCAGCAAAGAGTCTATCCT
GCCCAAGAGGAACAGCGATAAGCTGATCGCCAGAAAGAAGGACTGGGACCCTAAGAAGTACGGCGGCT
TCGACAGCCCCACCGTGGCCTATTCTGTGCTGGTGGTGGCCAAAGTGGAAAAGGGCAAGTCCAAGAAA
CTGAAGAGTGTGAAAGAGCTGCTGGGGATCACCATCATGGAAAGAAGCAGCTTCGAGAAGAATCCCAT
CGACTTTCTGGAAGCCAAGGGCTACAAAGAAGTGAAAAAGGACCTGATCATCAAGCTGCCTAAGTACT
CCCTGTTCGAGCTGGAAAACGGCCGGAAGAGAATGCTGGCCTCTGCCGGCGAACTGCAGAAGGGAAAC
GAACTGGCCCTGCCCTuCAAATATGTGAACTTCCTGTACCTGGCCAGCCACTATGAGAAGCTGAAGGG
CTCCCCCGAGGATAATGAGCAGAAACAGCTGTTTGTGGAACAGCACAAGCACTACCTGGACGAGATCA
TCGAGCAGATCAGCGAGTTCTCCAAGAGAGTGATCCTGGCCGACGCTAATCTGGACAAAGTGCTGTCC
GCCTACAACAAGCACCGGGATAAGCCCATCAGAGAGCAGGCCGAGAATATCATCCACCTGTTTACCCT
GACCAATCTGGGAGCCCCTGCCGCCTTCAAGTACTTTGACACCACCATCGACCGGAAGAGGTACACCA
GCACCAAAGAGGTGCTGGACGCCACCCTGATCCACCAGAGCATCACCGGCCTGTACGAGACACGGATC
GACCTGTCTCAGCTGGGAGGCGACaagcgacctgccgccacaaagaaggctggacaggctaagaagaa
gaaactggactctggaggatccgactacaaagaccatgacggtgattataaagatcatgacatcgatt
acaaggatgacgatgacaagggaggatccaaggagaagagtgcttgtcctaaagatccagccaaacct
ccggccaaggcacaagttgtgggatggccaccggtgagatcataccggaagaacgtgatggtttcctg
ccaaaaatcaagcggtggcccggaggcggcggcgttcgtgaaggtatcaatggacggagcaccgtact
tgaggaaaatcgatttgaggatgtataaaggcggatctggcggctctggaggatccattttcaaacca
gaagaactacgacaggcactgatgccaactttggaggcactttaccgtcaggatccagaatcccttcc
ctttcgtcaacctgtggaccctcagcttttaggaatccctgattactttgatattgtgaagagcccca
tggatctttctaccattaagaggaagttagacactggacagtatcaggagccctggcagtatgtcgat
gatatttggcttatgttcaataatgcctggttatataaccggaaaacatcacgggtatacaaatactg
ctccaagctctctgaggtctttgaacaagaaattgacccagtgatgcaaagccttggatactgttgtg
gcagaaagttggagttctctccacagacactgtgttgctacggcaaacagttgtgcacaatacctcgt
gatgccacttattacagttaccagaacaggtatcatttctgtgagaagtgtttcaatgagatccaagg
ggagagcgtttctttgggggatgacccttcccagcctcaaactacaataaataaagaacaattttcca
agagaaaaaatgacacactggatcctgaactgtttgttgaatgtacagagtgcggaagaaagatgcat
cagatctgtatccttcaccatgagatcatctggcctgctggattcgtctgtgatggctgtttaaagaa
aagtgcacgaactaggaaagaaaataagttttctgctaaaaggttgccatctaccagacttggcacct
ttctagagaatcgtgtgaatgactttctgaggcgacagaatcaccctgagtcaggagaggtcactgtt
agagtagttcatgcttctgacaaaaccgtggaagtaaaaccaggcatgaaagcaaggtttgtggacag
tggagagatggcagaatcctttccataccgaaccaaagccctctttgcctttgaagaaattgatggtg
ttgacctgtgcttctttggcatgcatgttcaagagtatggctctgactgccctccacccaaccagagg
agagtatacatatcttacctcgatagtgttcatttcttccgtcctaaatgcttgaggactgcagtcta
tcatgaaatcctaattggatatttagaatatgtcaagaaattaggttacacaacagggcatatttggg
catgtccaccaagtgagggagatgattatatcttccattgccatcctcctgaccagaagatacccaag
cccaagcgactgcaggaatggtacaaaaaaatgcttgacaaggctgtatcagagcgtattgtccatga
ctacaaggatatttttaaacaagctactgaagatagattaacaagtgcaaaggaattgccttatttcg
agggtgatttctggcccaatgttctggaagaaagcattaaggaactggaacaggaggaagaagagaga
aaacgagaggaaaacaccagcaatgaaagcacagatgtgaccaagggagacagcaaaaatgctaaaaa
gaagaataataagaaaaccagcaaaaataagagcagcctgagtaggggcaacaagaagaaacccggga
tgcccaatgtatctaacgacctctcacagaaactatatgccaccatggagaagcataaagaggtcttc
tttgtgatccgcctcattgctggccctgctgccaactccctgcctcccattgttgatcctgatcctct
catcccctgcgatctgatggatggtcgggatgcgtttctcacgctggcaagggacaagcacctggagt
tctcttcactccgaagagcccagtggtccaccatgtgcatgctggtggagctgcacacgcagagccag
gactga
Amino acid sequence fordCas9-p300c
(corresponding to SEQ ID NO: 79); lowercase
underlined = p300c; capital underlined = dCas9.
SEQ ID NO: 80
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTTY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKKLEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDkrpaatkkaqqakkkkldsggsdykdhdgdykdhdidykddddkggskeksacpkdpakp
pakaqvvgwppvrsyrknvmvscqkssggpeaaafvkvsmdgapylrkidlrmykggsggsggsifkp
eelrqalmptlealyrqdpeslprfqpvapqllgipdyfdivkspmdlstlkrkldtgqyqapwqyvd
diwlmfnnawlynrktsrvykycsklsevfeqeidpvmgslgyccgrklefspqtlccyqkqlctipr
datyysyqnryhfcekcfneiqgesvslgddpsqpqttinkeqfskrkndtldpelfvectecgrkmh
qicvlhheiiwpagfvcdgclkksartrkenkfsakrlpstrlgtflenrvndfIrrqnhpesgevtv
rvvhasdktvevkpgmkarfvdsgemaesfpyrtkalfafeeidgvdlcffgmhvqeygsdcpppngr
rvyisyldsvhffrpkclrtavyheiligyleyvkklgyttghiwacppsegddyifhchppdqkipk
pkrlqewykkmldkavserivhdykdifkqatedrltsakeipyfeqdfwpnvleesikelegeeeer
kreentsnestdvtkgdsknakkknnkktsknksslsrgnkkkpgmpnvsndlsqklyatmekhkevf
fvirliagpaanslppivdpdplipcdlmdgrdafltlardkhlefsslrraqwstmcmlvelhtqsq
d*
DNA sequence for dCas9-p300c (in backbone pNI123): pNH33; lowercase
underlined = p300c; capital underlined = dCas9.
SEQ ID NO: 81
ATGGACAAGAAGTACTCCATTGGGCTCGCCATCGGCACAAACAGCGTCGGCTGGGCCGTCATTACGGA
CGAGTACAAGGTGCCGAGCAAAAAATTCAAAGTTCTGGGCAATACCGATCGCCACAGCATAAAGAAGA
ACCTCATTGGCGCCCTCCTGTTCGACTCCGGGGAAACCGCCGAAGCCACGCGGCTCAAAAGAACAGCA
CGGCGCAGATATACCCGCAGAAAGAATCGGATCTGCTACCTGCAGGAGATCTTTAGTAATGAGATGGC
TAAGGTGGATGACTCTTTCTTCCATAGGCTGGAGGAGTCCTTTTTGGTGGAGGAGGATAAAAAGCACG
AGCGCCACCCAATCTTTGGCAATATCGTGGACGAGGTGGCGTACCATGAAAAGTACCCAACCATATAT
CATCTGAGGAAGAAGCTTGTAGACAGTACTGATAAGGCTGACTTGCGGTTGATCTATCTCGCGCTGGC
GCATATGATCAAATTTCGGGGACACTTCCTCATCGAGGGGGACCTGAACCCAGACAACAGCGATGTCG
ACAAACTCTTTATCCAACTGGTTCAGACTTACAATCAGCTTTTCGAAGAGAACCCGATCAACGCATCC
GGAGTTGACGCCAAAGCAATCCTGAGCGCTAGGCTGTCCAAATCCCGGCGGCTCGAAAACCTCATCGC
ACAGCTCCCTGGGGAGAAGAAGAACGGCCTGTTTGGTAATCTTATCGCCCTGTCACTCGGGCTGACCC
CCAACTTTAAATCTAACTTCGACCTGGCCGAAGATGCCAAGCTTCAACTGAGCAAAGACACCTACGAT
GATGATCTCGACAATCTGCTGGCCCAGATCGGCGACCAGTACGCAGACCTTTTTTTGGCGGCAAAGAA
CCTGTCAGACGCCATTCTGCTGAGTGATATTCTGCGAGTGAACACGGAGATCACCAAAGCTCCGCTGA
GCGCTAGTATGATCAAGCGCTATGATGAGCACCACCAAGACTTGACTTTGCTGAAGGCCCTTGTCAGA
CAGCAACTGCCTGAGAAGTACAAGGAAATTTTCTTCGATCAGTCTAAAAATGGCTACGCCGGATACAT
TGACGGCGGAGCAAGCCAGGAGGAATTTTACAAATTTATTAAGCCCATCTTGGAAAAAATGGACGGCA
CCGAGGAGCTGCTGGTAAAGCTTAACAGAGAAGATCTGTTGCGCAAACAGCGCACTTTCGACAATGGA
AGCATCCCCCACCAGATTCACCTGGGCGAACTGCACGCTATCCTCAGGCGGCAAGAGGATTTCTACCC
CTTTTTGAAAGATAACAGGGAAAAGATTGAGAAAATCCTCACATTTCGGATACCCTACTATGTAGGCC
CCCTCGCCCGGGGAAATTCCAGATTCGCGTGGATGACTCGCAAATCAGAAGAGACCATCACTCCCTGG
AACTTCGAGGAAGTCGTGGATAAGGGGGCCTCTGCCCAGTCCTTCATCGAAAGGATGACTAACTTTGA
TAAAAATCTGCCTAACGAAAAGGTGCTTCCTAAACACTCTCTGCTGTACGAGTACTTCACAGTTTATA
ACGAGCTCACCAAGGTCAAATACGTCACAGAAGGGATGAGAAAGCCAGCATTCCTGTCTGGAGAGCAG
AAGAAAGCTATCGTGGACCTCCTCTTCAAGACGAACCGGAAAGTTACCGTGAAACAGCTCAAAGAAGA
CTATTTCAAAAAGATTGAATGTTTCGACTCTGTTGAAATCAGCGGAGTGGAGGATCGCTTCAACGCAT
CCCTGGGAACGTATCACGATCTCCTGAAAATCATTAAAGACAAGGACTTCCTGGACAATGAGGAGAAC
GAGGACATTCTTGAGGACATTGTCCTCACCCTTACGTTGTTTGAAGATAGGGAGATGATTGAAGAACG
CTTGAAAACTTACGCTCATCTCTTCGACGACAAAGTCATGAAACAGCTCAAGAGGCGCCGATATACAG
GATGGGGGCGGCTGTCAAGAAAACTGATCAATGGGATCCGAGACAAGCAGAGTGGAAAGACAATCCTG
GATTTTCTTAAGTCCGATGGATTTGCCAACCGGAACTTCATGCAGTTGATCCATGATGACTCTCTCAC
CTTTAAGGAGGACATCCAGAAAGCACAAGTTTCTGGCCAGGGGGACAGTCTTCACGAGCACATCGCTA
ATCTTGCAGGTAGCCCAGCTATCAAAAAGGGAATACTGCAGACCGTTAAGGTCGTGGATGAACTCGTC
AAAGTAATGGGAAGGCATAAGCCCGAGAATATCGTTATCGAGATGGCCCGAGAGAACCAAACTACCCA
GAAGGGACAGAAGAACAGTAGGGAAAGGATGAAGAGGATTGAAGAGGGTATAAAAGAACTGGGGTCCC
AAATCCTTAAGGAACACCCAGTTGAAAACACCCAGCTTCAGAATGAGAAGCTCTACCTGTACTACCTG
CAGAACGGCAGGGACATGTACGTGGATCAGGAACTGGACATCAATCGGCTCTCCGACTACGACGTGGA
TGCCATCGTGCCCCAGTCTTTTCTCAAAGATGATTCTATTGATAATAAAGTGTTGACAAGATCCGATA
AAAATAGAGGGAAGAGTGATAACGTCCCCTCAGAAGAAGTTGTCAAGAAAATGAAAAATTATTGGCGG
CAGCTGCTGAACGCCAAACTGATCACACAACGGAAGTTCGATAATCTGACTAAGGCTGAACGAGGTGG
CCTGTCTGAGTTGGATAAAGCCGGCTTCATCAAAAGGCAGCTTGTTGAGACACGCCAGATCACCAAGC
ACGTGGCCCAAATTCTCGATTCACGCATGAACACCAAGTACGATGAAAATGACAAACTGATTCGAGAG
GTGAAAGTTATTACTCTGAAGTCTAAGCTGGTCTCAGATTTCAGAAAGGACTTTCAGTTTTATAAGGT
GAGAGAGATCAACAATTACCACCATGCGCATGATGCCTACCTGAATGCAGTGGTAGGCACTGCACTTA
TCAAAAAATATCCCAAGCTTGAATCTGAATTTGTTTACGGAGACTATAAAGTGTACGATGTTAGGAAA
ATGATCGCAAAGTCTGAGCAGGAAATAGGCAAGGCCACCGCTAAGTACTTCTTTTACAGCAATATTAT
GAATTTTTTCAAGACCGAGATTACACTGGCCAATGGAGAGATTCGGAAGCGACCACTTATCGAAACAA
ACGGAGAAACAGGAGAAATCGTGTGGGACAAGGGTAGGGATTTCGCGACAGTCCGGAAGGTCCTGTCC
ATGCCGCAGGTGAACATCGTTAAAAAGACCGAAGTACAGACCGGAGGCTTCTCCAAGGAAAGTATCCT
CCCGAAAAGGAACAGCGACAAGCTGATCGCACGCAAAAAAGATTGGGACCCCAAGAAATACGGCGGAT
TCGATTCTCCTACAGTCGCTTACAGTGTACTGGTTGTGGCCAAAGTGGAGAAAGGGAAGTCTAAAAAA
CTCAAAAGCGTCAAGGAACTGCTGGGCATCACAATCATGGAGCGATCAAGCTTCGAAAAAAACCCCAT
CGACTTTCTCGAGGCGAAAGGATATAAAGAGGTCAAAAAAGACCTCATCATTAAGCTTCCCAAGTACT
CTCTCTTTGAGCTTGAAAACGGCCGGAAACGAATGCTCGCTAGTGCGGGCGAGCTGCAGAAAGGTAAC
GAGCTGGCACTGCCCTCTAAATACGTTAATTTCTTGTATCTGGCCAGCCACTATGAAAAGCTCAAAGG
GTCTCCCGAAGATAATGAGCAGAAGCAGCTGTTCGTGGAACAACACAAACACTACCTTGATGAGATCA
TCGAGCAAATAAGCGAATTCTCCAAAAGAGTGATCCTCGCCGACGCTAACCTCGATAAGGTGCTTTCT
GCTTACAATAAGCACAGGGATAAGCCCATCAGGGAGCAGGCAGAAAACATTATCCACTTGTTTACTCT
GACCAACTTGGGCGCGCCTGCAGCCTTCAAGTACTTCGACACCACCATAGACAGAAAGCGGTACACCT
CTACAAAGGAGGTCCTGGACGCCACACTGATTCATCAGTCAATTACGGGGCTCTATGAAACAAGAATC
GACCTCTCTCAGCTCGGTGGAGACagcagggctgaccccaagaagaagaggaaggtggctagcatttt
caaaccagaagaactacgacaggcactgatgccaactttggaggcactttaccgtcaggatccagaat
cccttccctttcgtcaacctgtggaccctcagcttttaggaatccctgattactttgatattgtgaag
agccccatggatctttctaccattaagaggaagttagacactggacagtatcaggagccctggcagta
tgtcgatgatatttggcttatgttcaataatgcctggttatataaccggaaaacatcacgggtataca
aatactgctccaagctctctgaggtctttgaacaagaaattgacccagtgatgcaaagccttggatac
tgttgtggcagaaagttggagttctctccacagacactgtgttgctacggcaaacagttgtgcacaat
acctcgtgatgccacttattacagttaccagaacaggtatcatttctgtgagaagtgtttcaatgaga
tccaaggggagagcgtttctttgggggatgacccttcccagcctcaaactacaataaataaagaacaa
ttttccaagagaaaaaatgacacactggatcctgaactgtttgttgaatgtacagagtgcggaagaaa
gatgcatcagatctgtgtccttcaccatgagatcatctggcctgctggattcgtctgtgatggctgtt
taaagaaaagtgcacgaactaggaaagaaaataagttttctgctaaaaggttgccatctaccagactt
ggcacctttctagagaatcgtgtgaatgactttctgaggcgacagaatcaccctgagtcaggagaggt
cactgttagagtagttcatgcttctgacaaaaccgtggaagtaaaaccaggcatgaaagcaaggtttg
tggacagtggagagatggcagaatcctttccataccgaaccaaagccctctttgcctttgaagaaatt
gatggtgttgacctgtgcttctttggcatgcatgttcaagagtatggctctgactgccctccacccaa
ccagaggagagtatacatatcttacctcgatagtgttcatttcttccgtcctaaatgcttgaggactg
cagtctatcatgaaatcctaattggatatttagaatatgtcaagaaattaggttacacaacagggcat
atttgggcatgtccaccaagtgagggagatgattatatcttccattgccatcctcctgaccagaagat
acccaagcccaagcgactgcaggaatggtacaaaaaaatgcttgacaaggctgtatcagagcgtattg
tccatgactacaaggatatttttaaacaagctactgaagatagattaacaagtgcaaaggaattgcct
tatttcgagggtgatttctggcccaatgttctggaagaaagcattaaggaactggaacaggaggaaga
agagagaaaacgagaggaaaacaccagcaatgaaagcacagatgtgaccaagggagacagcaaaaatg
ctaaaaagaagaataataagaaaaccagcaaaaataagagcagcctgagtaggggcaacaagaagaaa
cccggatgcccaatgtatctaacgacctctccacagaaactatatgccaccatggagaagcataaaga
ggtcttctttgtgatccgcctcattgctggccctgctgccaactccctgcctcccattgttgatcctg
atcctctcatcccctgcgatctgatggatggtcgggatgcgtttctcacgctggcaagggacaagcac
ctggagttctcttcactccgaagagcccagtggtccaccatgtgcatgctggtggagctgcacacgca
gagccaggac
Amino acid sequence fordCas9-p300c
(corresponding to SEQ ID NO: 81); lowercase
underlined = p300c; capital underlined = dCas9.
SEQ ID NO: 82
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTTY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHOSITGLYETRI
DLSQLGGDSRADpkkkrkvasifkpeelrqalmptlealyrqdpeslpfrqpvdpqllgipdyfdivk
spmdlstikrkldtgqyqepwayvddiwlmfnnawlynrktsrvykycsklsevfeceidpvmgslqy
ccgrklefspqtlccygkqlctiprdatyysyqnryhfcekcfneiqgesvsIgddpsqpqttinkeq
fskrkndtldpelfvectecgrkmhqicvlhheiiwpagfvcdgclkksartrkenkfsakrlpstrl
gtflenrvndfirrgnhpesgevtvrvvhasdktvevkpgmkarfvdsgemaesfpyrtkalfafeei
davdlcffqmhvqeygsdcpppngrrvyisyldsvhffrpkclrtavyheiligyleyvkklgyttch
iwacppsegddyifhchppdqkipkpkrlqewykkmldkavserivhdykdifkqatedrltsakelp
yfegdfwpnvleesikeleqeeeerkreentsnestdvtkgdsknakkknnkktsknksslsrgnkkk
pgmpnvsndisqklyatmekhkevffvirliagpaanslppivdpdplipcdlmdgrdafltlardkh
lefsslrraqwstmcmlvelhtqsqd
DNA sequence for P300c-dCas9 (in backbone pNI36):
pN197; lowercase underlined = p300c;
capital underlined = dCas9,
SEQ ID NO: 83
atgggtattttcaaaccagaagaactacgacaggcactgatgccaactttggaggcactttaccgtca
ggatccagaatcccttccctttcgtcaacctgtggaccctcagcttttaggaatccctgattactttg
atattgtgaagagccccatggatctttctaccattaagaggaagttagacactggacagtatcaggag
ccctggcagtatgtcgatgatatttggcttatgttcaataatgcctggttatataaccggaaaacatc
acgggtatacaaatactgctccaagctctctgaggtctttgaacaagaaattgacccagtgatgcaaa
gccttggatactgttgtggcagaaagttggagttctctccacagacactgtgttgctacggcaaacag
ttgtgcacaatacctcgtgatgccacttattacagttaccagaacaggtatcatttctgtgagaagtg
tttcaatgagatccaaggggagagcgtttctttgggggatgacccttcccagcctcaaactacaataa
ataaagaacaattttccaagagaaaaaatgacacactggatcctgaactgtttgttgaatgtacagag
tgcggaagaaagatgcatcagatctgtgtccttcaccatgagatcatctggcctgctggattcgtctg
tgatggctgtttaaagaaaagtgcacgaactaggaaagaaaataagttttctgctaaaaggttgccat
ctaccagacttggcacctttctagagaatcgtgtgaatgactttctgaggcgacagaatcaccctgag
tcaggagaggtcactgttagagtagttcatgcttctgacaaaaccgtggaagtaaaaccaggcatgaa
agcaaggtttgtggacagtggagagatggcagaatcctttccataccgaaccaaagccctctttgcct
ttgaagaaattgatggtgttgacctgtgcttctttggcatgcatgttcaagagtatggctctgactgc
cctccacccaaccagaggagagtatacatatcttacctcgatagtgttcatttcttccgtcctaaatg
cttgaggactgcagtctatcatgaaatcctaattggatatttagaatatgtcaagaaattaggttaca
caacagggcatatttgggcatgtccaccaagtgagggagatgattatatcttccattgccatcctcct
gaccagaagatacccaagcccaagcgactgcaggaatggtacaaaaaaatgcttgacaaggctgtatc
agagcgtattgtccatgactacaaggatatttttaaacaagctactgaagatagattaacaagtgcaa
aggaattgccttatttcgagggtgatttctggcccaatgttctggaagaaagcattaaggaactggaa
caggaggaagaagagagaaaacgagaggaaaacaccagcaatgaaagcacagatgtgaccaagggaga
cagcaaaaatgctaaaaagaagaataataagaaaaccagcaaaaataagagcagcctgagtaggggca
acaagaagaaacccgggatgcccaatgtatctaacgacctctcacagaaactatatgccaccatggag
aagcataaagaggtcttctttgtgatccgcctcattgctggccctgctgccaactccctgcctcccat
tgttgatcctgatcctctcatcccctgcgatctgatggatggtcgggatgcgtttctcacgctggcaa
gggacaagcacctggagttctcttcactccgaagagcccagtggtccaccatgtgcatgctggtggag
ctgcacacgcagagccaggacggctctggaggatctggcggctctagcgccaccATGGACAAGAAGTA
CAGCATCGGCCTGGCCATCGGCACCAACTCTGTGGGCTGGGUCGTGATCACCGACGAGTACAAGGTGC
CCAGCAAGAAATTCAAGGTGCTGGGCAACACCGACCGGCACAGCATCAAGAAGAACCTGATCGGAGCC
CTGCTGTTCGACAGCGGCGAAACAGCCGAGGCCACCCGGCTGAAGAGAACCGCCAGAAGAAGATACAC
CAGACGGAAGAACCGGATCTGCTATCTGCAAGAGATCTTCAGCAACGAGATGGCCAAGGTGGACGACA
GCTTCTTCCACAGACTGGAAGAGTCCTTCCTGGTGGAAGAGGATAAGAAGCACGAGCGGCACCCCATC
TTCGGCAACATCGTGGACGAGGTGGCCTACCACGAGAAGTACCCCACCATCTACCACCTGAGAAAGAA
ACTGGTGGACAGCACCGACAAGGCCGACCTGCGGCTGATCTATCTGGCCCTGGCCCACATGATCAAGT
TCCGGGGCCACTTCCTGATCGAGGGCGACCTGAACCCCGACAACAGCGACGTGGACAAGCTGTTCATC
CAGCTGGTGCAGACCTACAACCAGCTGTTCGAGGAAAACCCCATCAACGCCAGCGGCGTGGACGCCAA
GGCCATCCTGTCTGCCAGACTGAGCAAGAGCAGACGGCTGGAAAATCTGATCGCCCAGCTGCCCGGCG
AGAAGAAGAATGGCCTGTTCGGAAACCTGATTGCCCTGAGCCTGGGCCTGACCCCCAACTTCAAGAGC
AACTTCGACCTGGCCGAGGATGCCAAACTGCAGCTGAGCAAGGACACCTACGACGACGACCTGGACAA
CCTGCTGGCCCAGATCGGCGACCAGTACGCCGACCTGTTTCTGGCCGCCAAGAACCTGTCCGACGCCA
TCCTGCTGAGCGACATCCTGAGAGTGAACACCGAGATCACCAAGGCCCCCCTGAGCGCCTCTATGATC
AAGAGATACGACGAGCACCACCAGGACCTGACCCTGCTGAAAGCTCTCGTGCGGCAGCAGCTGCCTGA
GAAGTACAAAGAGATTTTCTTCGACCAGAGCAAGAACGGCTACGCCGGCTACATTGACGGCGGAGCCA
GCCAGGAAGAGTTCTACAAGTTCATCAAGCCCATCCTGGAAAAGATGGACGGCACCGAGGAACTGCTC
GTGAAGCTGAACAGAGAGGACCTGCTGCGGAAGCAGCGGACCTTCGACAACGGCAGCATCCCCCACCA
GATCCACCTGGGAGAGCTGCACGCCATTCTGCGGCGGCAGGAAGATTTTTACCCATTCCTGAAGGACA
ACCGGGAAAAGATCGAGAAGATCCTGACCTTCCGCATCCCCTACTACGTGGGCCCTCTGGCCAGGGGA
AACAGCAGATTCGCCTGGATGACCAGAAAGAGCGAGGAAACCATCACCCCCTGGAACTTCGAGGAAGT
GGTGGACAAGGGCGCTTCCGCCCAGAGCTTCATCGAGCGGATGACCAACTTCGATAAGAACCTGCCCA
ACGAGAAGGTGCTGCCCAAGCACAGCCTGCTGTACGAGTACTTCACCGTGTATAACGAGCTGACCAAA
GTGAAATACGTGACCGAGGGAATGAGAAAGCCCGCCTTCCTGAGCGGCGAGCAGAAAAAGGCCATCGT
GGACCTGCTGTTCAAGACCAACCGGAAAGTGACCGTGAAGCAGCTGAAAGAGGACTACTTCAAGAAAA
TCGAGTGCTTCGACTCCGTGGAAATCTCCGGCGTGGAAGATCGGTTCAACGCCTCCCTGGGCACATAC
CACGATCTGCTGAAAATTATCAAGGACAAGGACTTCCTGGACAATGAGGAAAACGAGGACATTCTGGA
AGATATCGTGCTGACCCTGACACTGTTTGAGGACAGAGAGATGATCGAGGAACGGCTGAAAACCTATG
CCCACCTGTTCGACGACAAAGTGATGAAGCAGCTGAAGCGGCGGAGATACACCGGCTGGGGCAGGCTG
AGCCGGAAGCTGATCAACGGCATCCGGGACAAGCAGTCCGGCAAGACAATCCTGGATTTCCTGAAGTC
CGACGGCTTCGCCAACAGAAACTTCATGCAGCTGATCCACGACGACAGCCTGACCTTTAAAGAGGACA
TCCAGAAAGCCCAGGTGTCCGGCCAGGGCGATAGCCTGCACGAGCACATTGCCAATCTGGCCGGCAGC
CCCGCCATTAAGAAGGGCATCCTGCAGACAGTGAAGGTGGTGGACGAGCTCGTGAAAGTGATGGGCCG
GCACAAGCCCGAGAACATCGTGATCGAAATGGCCAGAGAGAACCAGACCACCCAGAAGGGACAGAAGA
ACAGCCGCGAGAGAATGAAGCGGATCGAAGAGGGCATCAAAGAGCTGuGCAGCCAGATCCTGAAAGAA
CACCCCGTGGAAAACACCCAGCTGCAGAACGAGAAGCTGTACCTGTACTACCTGCAGAATGGGCGGGA
TATGTACGTGGACCAGGAACTGGACATCAACCGGCTGTCCGACTACGATGTGGACGCCATCGTGCCTC
AGAGCTTTCTGAAGGACGACTCCATCGACAACAAGGTGCTGACCAGAAGCGACAAGAACCGGGGCAAG
AGCGACAACGTGCCCTCCGAAGAGGTCGTGAAGAAGATGAAGAACTACTGGCGGCAGCTGCTGAACGC
CAAGCTGATTACCCAGAGAAAGTTCGACAATCTGACCAAGGCCGAGAGAGGCGGCCTGAGCGAACTGG
ATAAGGCCGGCTTCATCAAGAGACAGCTGGTGGAAACCCGGCAGATCACAAAGCACGTGGCACAGATC
CTGGACTCCCGGATGAACACTAAGTACGACGAGAATGACAAGCTGATCCGGGAAGTGAAAGTGATCAC
CCTGAAGTCCAAGCTGGTGTCCGATTTCCGGAAGGATTTCCAGTTTTACAAAGTGCGCGAGATCAACA
ACTACCACCACGCCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGATGATCGCCAAGAG
CGAGCAGGAAATCGGCAAGGCTACCGCCAAGTACTTCTTCTACAGCAACATCATGAACTTTTTCAAGA
CCGAGATTACCCTGGCCAACGGCGAGATCCGGAAGCGGCCTCTGATCGAGACAAACGGCGAAACCGGG
GAGATCGTGTGGGATAAGGGCCGGGATTTTGCCACCGTGCGGAAAGTGCTGAGCATGCCCCAAGTGAA
TATCGTGAAAAAGACCGAGGTGCAGACAGGCGGCTTCAGCAAAGAGTCTATCCTGCCCAAGAGGAACA
GCGATAAGCTGATCGCCAGAAAGAAGGACTGGGACCCTAAGAAGTACGGCGGCTTCGACAGCCCCACC
GTGGCCTATTCTGTGCTGGTGGTGGCCAAAGTGGAAAAGGGCAAGTCCAAGAAACTGAAGAGTGTGAA
AGAGCTGCTGGGGATCACCATCATGGAAAGAAGCAGCTTCGAGAAGAATCCCATCGACTTTCTGGAAG
CCAAGGGCTACAAAGAAGTGAAAAAGGACCTGATCATCAAGCTGCCTAAGTACTCCCTGTTCGAGCTG
GAAAACGGCCGGAAGAGAATGCTGGCCTCTGCCGGCGAACTGCAGAAGGGAAACGAACTGGCCCTGCC
CTCCAAATATGTGAACTTCCTGTACCTGGCCAGCCACTATGAGAAGCTGAAGGGCTCCCCCGAGGATA
ATGAGCAGAAACAGCTGTTTGTGGAACAGCACAAGCACTACCTGGACGAGATCATCGAGCAGATCAGC
GAGTTCTCCAAGAGAGTGATCCTGGCCGACGCTAATCTGGACAAAGTGCTGTCCGCCTACAACAAGCA
CCGGGATAAGCCCATCAGAGAGCAGGCCGAGAATATCATCCACCTGTTTACCCTGACCAATCTGGGAG
CCCCTGCCGCCTTCAAGTACTTTGACACCACCATCGACCGGAAGAGGTACACCAGCACCAAAGAGGTG
CTGGACGCCACCCTGATCCACCAGAGCATCACCGGCCTGTACGAGACACGGATCGACCTGTCTCAGCT
GGGAGGCGAC
Amino acid sequence for P300c-dCas9
(corresponding to SEQ ID NO: 83); lowercase
underlined = p300c; capital underlined = dCas9.
SEQ ID NO: 84
mqifkpeelrqalmptlealyrqdpeslpfrqpvdpqllgipdyfdivkspmdlstikrkldtgqyge
pwqyvddiwlmfnnawlynrktsrvykycsklsevfeqeidpvmgslgycogrklefspgtlccyqkq
lctiprdatyysygnryhfcekcfneiqgesvslgddpsgpqllinkeqfskrkndlldpelfvecte
cgrkmhqicvlhheiiwpagfvcdgcikksartrkenkfsakrlpstrlgtflenrvndflrrqnhpe
sqevtvryvhasdktvevkpgmkarfvdsgemaesfpyrtkalfafeeidgvdlcffgmhvqeygsdc
pppnqrrvyisyldsvhffrpkclrtavyheiligyleyvkklgyttghiwacppsegddyifhchpp
dqkipkpkrlqewykkmldkavserivhdykdifkgatedrltsakelpyfegdfwpnvleesikele
qeeeerkreentsnestdvtkgdsknakkknnkktsknkssisrgnkkkpgmpnvsndlsqklvatme
khkevffvirliagpaanslppivdpdplipcdlmdgrdafltlardkhlefsslrraqwstmcmlve
lhtqsqdgsgasggssatMDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIPYYVGPLARG
NSRFAWMTRKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQLKRRRYTGWGRL
SRKLINGIRDKQSGKTILDFLKSDGFANRNFMQLIHDDSLTFKEDIQKAQVSGQGDSLHEHIANLAGS
PAIKKGILQTVKVVDELVKVMGRHKPENIVIEMARENQTTQKGQKNSRERMKRIEEGIKELGSQILKE
HPVENTQLQNEKLYLYYLQNGRDMYVDQELDINRLSDYDVDAIVPQSFLKDDSIDNKVLTRSDKNRGK
SDNVPSEEVVKKMKNYWRQLLNAKLITQRKFDNLTKAERGGLSELDKAGFIKRQLVETRQITKHVAQI
LDSRMNTKYDENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYFDTTIDRKRYTSTKEV
LDATLIHQSITGLYETRIDLSQLGGD
DNA sequence for P3000-dCas9 (in backbone pNH 23): pNH32; lowercase
underlined = p300c; capital underlined = dCas9.
SEQ ID NO: 85
atggactacaaagaccatgacggtgattataaagatcatgacatcgattacaaggatgacgatgacaa
gcacgttattttcaaaccagaagaactacgacaggcactgatgccaactttggaggcactttaccgtc
aggatccagaatcccttccctttcgtcaacctgtggaccatcagcttttaggaatccctgattacttt
gatattgtgaagagccccatggatctttctaccattaagaggaagttagacactggacagtatcagga
gccctggcagtatgtcgatgatatttggcttatgttcaataatgcctggttatataaccggaaaacat
cacgggtatacaaatactgctccaagctctctgaggtctttgaacaagaaattgacccagtgatgcaa
agccttggatactgttgtggcagaaagttggagttctctccacagacactgtgttgctacggcaaaca
gttgtgcacaatacctcgtgatgccacttattacagttaccagaacaggtatcatttctgtgagaagt
gtttcaatgagatccaaggggagagcgtttctttgggggatgacccttcccagcctcaaactacaata
aataaagaacaattttccaagagaaaaaatgacacactggatcctgaactgtttgttgaatgtacaga
gtgcggaagaaagatgcatcagatctgtgtcattcaccatgagatcatctggcctgctggattcgtct
gtgatggctgtttaaagaaaagtgcacgaactaggaaagaaaataagttttctgctaaaaggttgcca
tctaccagacttggcacctttctagagaatcgtgtgaatgactttctgaggcgacagaatcaccctga
gtcaggagaggtcactgttagagtagttcatgcttctgacaaaaccgtggaagtaaaaccaggcatga
aagcaaggtttgtggacagtggagagatggcagaatcctttccataccgaaccaaagccctctttgcc
tttgaagaaattgatggtgttgacctgtgcttctttggcatgcatgttcaagagtatggctctgactg
ccctccacccaaccagaggagagtatacatatcttacctcgatagtgttcatttcttccgtcctaaat
gcttgaggactgcagtctatcatgaaatcctaattggatatttagaatatgtcaagaaattaggttac
acaacagggcatatttgggcatgtccaccaagtgagggagatgattatatcttccattgccatcctcc
tgaccagaagatacccaagcccaagcgactgcaggaatggtacaaaaaaatgcttgacaaggctgtat
cagagcgtattgtccatgactacaaggatatttttaaacaagctactgaagatagattaacaagtgca
aaggaattgccttatttcgagggtgatttctggcccaatgttctggaagaaagcattaaggaactgga
acaggagaaagaagaaagaaaacgagagaaaaacaccagcaataaaagcacagatgtaaccaaaagag
acagcaaaaatgctaaaaagaagaataataagaaaaccagcaaaaataagagcagcctgagtaggggc
aacaagaagaaacccgggatgcccaatgtatctaacgacctctcacagaaactatatgccaccatgga
gaagcataaagaggtcttctttgtgatccgcctcattgctggccctgctgccaactccctgcctccca
ttgttgatcctgatcctctcatcccctgcgatctgatggatggtcgggatgcgtttctcacgctggca
agggacaagcacctggagttctcttcactccgaagagcccagtggtccaccatgtgcatgctggtgga
gctgcacacgcagagccaggacaaccccaagaagaagaggaaggtgggccgcggaATGGACAAGAAGT
ACTCCATTGGGCTCGCCATCGGCACAAACAGCGTCGGCTGGGCCGTCATTACGGACGAGTACAAGGTG
CCGAGCAAAAAATTCAAAGTTCTGGGCAATACCGATCGCCACAGCATAAAGAAGAACCTCATTGGCGC
CCTCCTGTTCGACTCCGGGGAAACCGCCGAAGCCACGCGGCTCAAAAGAACAGCACGGCGCAGATATA
CCCGCAGAAAGAATCGGATCTGCTACCTGCAGGAGATCTTTAGTAATGAGATGGCTAAGGTGGATGAC
TCTTTCTTCCATAGGCTGGAGGAGTCCTTTTTGGTGGAGGAGGATAAAAAGCACGAGCGCCACCCAAT
CTTTGGCAATATCGTGGACGAGGTGGCGTACCATGAAAAGTACCCAACCATATATCATCTGAGGAAGA
AGCTTGTAGACAGTACTGATAAGGCTGACTTGCGGTTGATCTATCTCGCGCTGGCGCATATGATCAAA
TTTCGGGGACACTTCCTCATCGAGGGGGACCTGAACCCAGACAACAGCGATGTCGACAAACTCTTTAT
CCAACTGGTTCAGACTTACAATCAGCTTTTCGAAGAGAACCCGATCAACGCATCCGGAGTTGACGCCA
AAGCAATCCTGAGCGCTAGGCTGTCCAAATCCCGGCGGCTCGAAAACCTCATCGCACAGCTCCCTGGG
GAGAAGAAGAACGGCCTGTTTGGTAATCTTATCGCCCTGTCACTCGGGCTGACCCCCAACTTTAAATC
TAACTTCGACCTGGCCGAAGATGCCAAGCTTCAACTGAGCAAAGACACCTACGATGATGATCTCGACA
ATCTGCTGGCCCAGATCGGCGACCAGTACGCAGACCTTTTTTTGGCGGCAAAGAACCTGTCAGACGCC
ATTCTGCTGAGTGATATTCTGCGAGTGAACACGGAGATCACCAAAGCTCCGCTGAGCGCTAGTATGAT
CAAGCGCTATGATGAGCACCACCAAGACTTGACTTTGCTGAAGGCCCTTGTCAGACAGCAACTGCCTG
AGAAGTACAAGGAAATTTTCTTCGATCAGTCTAAAAATGGCTACGCCGGATACATTGACGGCGGAGCA
AGCCAGGAGGAATTTTACAAATTTATTAAGCCCATCTTGGAAAAAATGGACGGCACCGAGGAGCTGCT
GGTAAAGCTTAACAGAGAAGATCTGTTGCGCAAACAGCGCACTTTCGACAATGGAAGCATCCCCCACC
AGATTCACCTGGGCGAACTGCACGCTATCCTCAGGCGGCAAGAGGATTTCTACCCCTTTTTGAAAGAT
AACAGGGAAAAGATTGAGAAAATCCTCACATTTCGGATACCCTACTATGTAGGCCCCCTCGCCCGGGG
AAATTCCAGATTCGCGTGGATGACTCGCAAATCAGAAGAGACCATCACTCCCTGGAACTTCGAGGAAG
TCGTGGATAAGGGGGCCTCTGCCCAGTCCTTCATCGAAAGGATGACTAACTTTGATAAAAATCTGCCT
AACGAAAAGGTGCTTCCTAAACACTCTCTGCTGTACGAGTACTTCACAGTTTATAACGAGCTCACCAA
GGTCAAATACGTCACAGAAGGGATGAGAAAGCGAGCATTCCTGTCTGGAGAGCAGAAGAAAGCTATCG
TGGACCTCCTCTTCAAGACGAACCGGAAAGTTACCGTGAAACAGCTCAAAGAAGACTATTTCAAAAAG
ATTGAATGTTTCGACTCTGTTGAAATCAGCGGAGTGGAGGATCGCTTCAACGCATCCCTGGGAACGTA
TCACGATCTCCTGAAAATCATTAAAGACAAGGACTTCCTGGACAATGAGGAGAACGAGGACATTCTTG
AGGACATTGTCCTCACCCTTACGTTGTTTGAAGATAGGGAGATGATTGAAGAACGCTTGAAAACTTAC
GCTCATCTCTTCGACGACAAAGTCATGAAACAGCTCAAGAGGCGCCGATATACAGGATGGGGGCGGCT
GTCAAGAAAACTGATCAATGGGATCCGAGACAAGCAGAGTGGAAAGACAATCCTGGATTTTCTTAAGT
CCGATGGATTTGCCAACCGGAACTTCATGCAGTTGATCCATGATGACTCTCTCACCTTTAAGGAGGAC
ATCCAGAAAGCACAAGTTTCTGGCCAGGGGGACAGTCTTCACGAGCACATCGCTAATCTTGCAGGTAG
CCCAGCTATCAAAAAGGGAATACTGCAGACCGTTAAGGTCGTGGATGAACTCGTCAAAGTAATGGGAA
GGCATAAGCCCGAGAATATCGTTATCGAGATGGCCCGAGAGAACCAAACTACCCAGAAGGGACAGAAG
AACAGTAGGGAAAGGATGAAGAGGATTGAAGAGGGTATAATGGAACTGGGGTCCCAAATCCTTAAGGA
ACACCCAGTTGAAAACACCCAGCTTCAGAATGAGAAGCTCTACCTGTACTACCTGCAGAACGGCAGGG
ACATGTACGTGGATCAGGAACTGGACATCAATCGGCTCTCCGACTACGACGTGGATGCCATCGTGCCC
CAGTCTTTTCTCAAAGATGATTCTATTGATAATAAAGTGTTGACAAGATCCGATAAAAATAGAGGGAA
GAGTGATAACGTCCCCTCAGAAGAAGTTGTCAAGAAAATGAAAAATTATTGGCGGCAGCTGCTGAACG
CCAAACTGATCACACAACGGAAGTTCGATAATCTGACTAAGGCTGAACGAGGTGGCCTGTCTGAGTTG
GATAAAGCCGGCTTCATCAAAAGGGAGCTTGTTGAGACACGCCAGATCACCAAGCACGTGGCCCAAAT
TCTCGATTCACGCATGAACACCAAGTACGATGAAAATGACAAACTGATTCGAGAGGTGAAAGTTATTA
CTCTGAAGTCTAAGCTGGTCTCAGATTTCAGAAAGGACTTTCAGTTTTATAAGGTGAGAGAGATCAAC
AATTACCACCATGCGCATGATGCCTACCTGAATGCAGTGGTAGGCACTGCACTTATCAAAAAATATCC
CAAGCTTGAATCTGAATTTGTTTACGGAGACTATAAAGTGTACGATGTTAGGAAAATGATCGCAAAGT
CTGAGCAGGAAATAGGCAAGGCCACCGCTAAGTACTTCTTTTACAGCAATATTATGAATTTTTTCAAG
ACCGAGATTACACTGGCCAATGGAGAGATTCGGAAGCGACCACTTATCGAAACAAACGGAGAAACAGG
AGAAATCGTGTGGGACAAGGGTAGGGATTTCGCGACAGTCCGGAAGGTCCTGTCCATGCCGCAGGTGA
ACATCGTTAAAAAGACCGAAGTACAGACCGGAGGCTTCTCCAAGGAAAGTATCCTCCCGAAAAGGAAC
AGCGACAAGCTGATCGCACGCAAAAAAGATTGGGACCCCAAGAAATACGGCGGATTCGATTCTCCTAC
AGTCGCTTACAGTGTACTGGTTGTGGCCAAAGTGGAGAAAGGGAAGTCTAAAAAACTCAAAAGCGTCA
AGGAACTGCTGGGCATCACAATCATGGAGCGATCAAGCTTCGAAAAAAACCCCATCGACTTTCTCGAG
GCGAAAGGATATAAAGAGGTCAAAAAAGACCTCATCATTAAGCTTCCCAAGTACTCTCTCTTTGAGCT
TGAAAACGGCCGGAAACGAATGCTCGCTAGTGCGGGCGAGCTGCAGAAAGGTAACGAGCTGGCACTGC
CCTCTAAATACGTTAATTTCTTGTATCTGGCCAGCCACTATGAAAAGCTCAAAGGGTCTCCCGAAGAT
AATGAGCAGAAGCAGCTGTTCGTGGAACAACACAAACACTACCTTGATGAGATCATCGAGCAAATAAG
CGAATTCTCCAAAAGAGTGATCCTCGCCGACGCTAACCTCGATAAGGTGCTTTCTGCTTACAATAAGC
ACAGGGATAAGCCCATCAGGGAGCAGGLAGAAAACATTATCCACTTGTTTACTCTGACCAACTTGGGC
GCGCCTGGAGCCTTGAAGTACTTGGACAGCACCATAGACAGAAAGGGGTACACCTCTACAAAGGAGGT
CCTGGACGCCACACTGATTCATCAGTCAATTACGGGGCTCTATGAAACAAGAATCGACCTCTCTCAGC
TCGGTGGAGACAGCAGGGCTGAC
Amino acid sequence for P300c-dCas9
(corresponding to SEQ ID NO: 85); lowercase
underlined = p300c; capital underlined = dCas9.
SEQ ID NO: 86
dykdhdgdykdhdidykddddkhvifkpeelrqalmpllealyrqdpeslpfrqpvdpqllqipdyfd
ivkspmdlstikrkldtgqyqepwayvddiwlmfnnawlynrktsrvykycsklsevfeqeidpvmgs
lgyccgrklefspqtlccygkqlctiprdatyysyqnryhfcekcfneiqgesvslgddpsqpgttin
keqfskrkndtldpelfvectecgrkmhqicvlhheiiwpagfvedgclkksartrkenkfsakrlps
trigtflenrvndfIrrqnhpesgevtvrvvhasdktvevkpgmkarfvdsgemaesfpyrtkalfaf
eeidgvdlcffgmhvqeygsdcpppngrrvyisyldsvhffrpkclrtavyheiligyleyvkklgyt
tghiwacppsegddyifhchppdqkipkpkrlqewykkmldkavserivhdykdifkqatedrltsak
elpyfegdfwpnvleesikeleqeeeerkreentsnestdvtkgdsknakkknnkktsknksslsrgn
kkkpgmpnvsndlsqklyatmekhkevffvirliaqpaanslppivdpdplipcdlmdgrdafltlar
dkhlefsslrraqwstmcmlvelhtqsqdnpkkkrkvgrgMDKKYSIGLAIGTNSVGWAVITDEYKVP
SKKFKVLGNTDRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYAGYIDGGAS
QEEFYKFIKPILEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYFKKI
ECFDSVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTFKEDI
QKAQVSGQGDSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSDYDVDAIVPQ
SFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKT
EITLANGEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLKGSPEDN
EQKQLFVEQHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSRAD
DNA sequence fordCas9-SS18 (in backbone pNI36):
pNI80; capital underlined = dCas9;
capital no underline = SS18
SEQ ID NO: 87
ATGGACAAGAAGTAGAGCATCGGCCTGGCCATCGGCACCAACTCTGTGGGCTGGGCCGTGATCACCGA
CGAGTACAAGGTGCCCAGCAAGAAATTCAAGGTGCTGGGCAACACCGACCGGCACAGCATCAAGAAGA
ACCTGATCGGAGCCCTGCTGTTCGACAGCGGCGAAACAGCCGAGGCCACCCGGCTGAAGAGAACCGCC
AGAAGAAGATACACCAGACGGAAGAACCGGATCTGCTATCTGCAAGAGATCTTCAGCAACGAGATGGC
CAAGGTGGACGACAGCTTCTTCCACAGACTGGAAGAGTCCTTCCTGGTGGAAGAGGATAAGAAGCACG
AGCGGCACCCCATCTTCGGCAACATCGTGGACGAGGTGGCCTACCACGAGAAGTACCCCACCATCTAC
CACCTGAGAAAGAAACTGGTGGACAGCACCGACAAGGCCGACCTGCGGCTGATCTATCTGGCCCTGGC
CCACATGATCAAGTTCCGGGGCCACTTCCTGATCGAGGGCGACCTGAACCCCGACAACAGCGACGTGG
ACAAGCTGTTCATCCAGCTGGTGCAGACCTACAACCAGCTGTTCGAGGAAAACCCCATCAACGCCAGC
GGCGTGGACGCCAAGGCCATCCTGTCTGCCAGACTGAGCAAGAGCAGACGGCTGGAAAATCTGATCGC
CCAGCTGCCCGGCGAGAAGAAGAATGGCCTGTTCGGAAACCTGATTGCCCTGAGCCTGGGCCTGACCC
CCAACTTCAAGAGCAACTTCGACCTGGCCGAGGATGCCAAACTGCAGCTGAGCAAGGACACCTACGAC
GACGACCTGGACAACCTGCTGGCCCAGATCGGCGACCAGTACGCCGACCTGTTTCTGGCCGCCAAGAA
CCTGTCCGACGCCATCCTGCTGAGCGACATCCTGAGAGTGAACACCGAGATCACCAAGGCCCCCCTGA
GCGCCTCTATGATCAAGAGATACGACGAGCACCACCAGGACCTGACCCTGCTGAAAGCTCTCGTGCGG
CAGCAGCTGCCTGAGAAGTACAAAGAGATTTTCTTCGACCAGAGCAAGAACGGCTACGCCGGCTACAT
TGACGGCGGAGCCAGCCAGGAAGAGTTCTACAAGTTCATCAAGCCCATCCTGGAAAAGATGGACGGCA
CCGAGGAACTGCTCGTGAAGCTGAACAGAGAGGACCTGCTGCGGAAGCAGCGGACCTTCGACAACGGC
AGCATCCCCCACCAGATCCACCTGGGAGAGCTGCACGCCATTCTGCGGCGGCAGGAAGATTTTTACCC
ATTCCTGAAGGACAACCGGGAAAAGATCGAGAAGATCCTGACCTTCCGCATCCCCTACTACGTGGGCC
CTCTGGCCAGGGGAAACAGCAGATTCGCCTGGATGACCAGAAAGAGCGAGGAAACCATCACCCCCTGG
AACTTCGAGGAAGTGGTGGACAAGGGCGCTTCCGCCCAGAGCTTCATCGAGCGGATGACCAACTTCGA
TAAGAACCTGCCCAACGAGAAGGTGCTGCCCAAGCACAGCCTGCTGTACGAGTACTTCACCGTGTATA
ACGAGCTGACCAAAGTGAAATACGTGACCGAGGGAATGAGAAAGCCCGCCTTCCTGAGCGGCGAGCAG
AAAAAGGCCATCGTGGACCTGCTGTTCAAGACCAACCGGAAAGTGACCGTGAAGCAGCTGAAAGAGGA
CTACTTCAAGAAAATCGAGTGCTTCGACTCCGTGGAAATCTCCGGCGTGGAAGATCGGTTCAACGCCT
CCCTGGGCACATACCACGATCTGCTGAAAATTATCAAGGACAAGGACTTCCTGGACAATGAGGAAAAC
GAGGACATTCTGGAAGATATCGTGCTGACCCTGACACTGTTTGAGGACAGAGAGATGATCGAGGAACG
GCTGAAAACCTATGCCCACCTGTTCGACGACAAAGTGATGAAGCAGCTGAAGCGGCGGAGATACACCG
GCTGGGGCAGGCTGAGCCGGAAGCTGATCAACGGCATCCGGGACAAGCAGTCCGGLAAGACAATCCTG
GATTTCCTGAAGTCCGACGGCTTCGCCAACAGAAACTTCATGCAGCTGATCCACGACGACAGCCTGAC
CTTTAAAGAGGACATCCAGAAAGCCCAGGTGTCCGGCCAGGGCGATAGCCTGCACGAGCACATTGCCA
ATCTGGCCGGCAGCCCCGCCATTAAGAAGGGCATCCTGCAGACAGTGAAGGTGGTGGACGAGCTCGTG
AAAGTGATGGGCCGGCACAAGCCCGAGAACATCGTGATCGAAATGGCCAGAGAGAACCAGACCACCCA
GAAGGGACAGAAGAACAGCCGCGAGAGAATGAAGCGGATCGAAGAGGGCATCAAAGAGCTGGGCAGCC
AGATCCTGAAAGAACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGACTACGATGTGGA
CGCCATCGTGCCTCAGAGCTTTCTGAAGGACGACTCCATCGACAACAAGGTGCTGACCAGAAGCGACA
AGAACCGGGGCAAGAGCGACAACGTGCCCTCCGAAGAGGTCGTGAAGAAGATGAAGAACTACTGGCGG
CAGCTGCTGAACGCCAAGCTGATTACCCAGAGAAAGTTCGACAATCTGACCAAGGCCGAGAGAGGCGG
CCTGAGCGAACTGGATAAGGCCGGCTTCATCAAGAGACAGCTGGTGGAAACCCGGCAGATCACAAAGC
ACGTGGCACAGATCCTGGACTCCCGGATGAACACTAAGTACGACGAGAATGACAAGCTGATCCGGGAA
GTGAAAGTGATCACCCTGAAGTCCAAGCTGGTGTCCGATTTCCGGAAGGATTTCCAGTTTTACAAAGT
GCGCGAGATCAACAACTAGCACCACGCCCACGACGCCTAGCTGAACGCCGTCGTGGGAACCGCCCTGA
TCAAAAAGTACCCTAAGCTGGAAAGCGAGTTCGTGTACGGCGACTACAAGGTGTACGACGTGCGGAAG
ATGATCGCCAAGAGCGAGCAGGAAATCGGCAAGuCTACCGCCAAGTACTTCTTCTACAGCAACATCAT
GAACTTTTTCAAGACCGAGATTACCCTGGCCAACGGCGAGATCCGGAAGCGGCCTCTGATCGAGACAA
ACGGCGAAACCGGGGAGATCGTGTGGGATAAGGGCCGGGATTTTGCCACCGTGCGGAAAGTGCTGAGC
ATGCCCCAAGTGAATATCGTGAAAAAGACCGAGGTGCAGACAGGCGGCTTCAGCAAAGAGTCTATCCT
GCCCAAGAGGAACAGCGATAAGCTGATCGCCAGAAAGAAGGACTGGGACCCTAAGAAGTACGGCGGCT
TCGACAGCCCCACCGTGGCCTATTCTGTGCTGGTGGTGGCCAAAGTGGAAAAGGGCAAGTCCAAGAAA
CTGAAGAGTGTGAAAGAGCTGCTGGGGATCACCATCATGGAAAGAAGCAGCTTCGAGAAGAATCCCAT
CGACTTTCTGGAAGCCAAGGGCTACAAAGAAGTGAAAAAGGACCTGATCATCAAGCTGCCTAAGTACT
CCCTGTTCGAGCTGGAAAACGGCCGGAAGAGAATGCTGGCCTCTGCCGGCGAACTGCAGAAGGGAAAC
GAACTGGCCCTGCCCTCCAAATATGTGAACTTCCTGTACCTGGCCAGCCACTATGAGAAGCTGAAGGG
CTCCCCCGAGGATAATGAGCAGAAACAGCTGTTTGTGGAACAGCACAAGCACTACCTGGACGAGATCA
TCGAGCAGATCAGCGAGTTCTCCAAGAGAGTGATCCTGGCCGACGCTAATCTGGACAAAGTGCTGTCC
GCCTACAACAAGCACCGGGATAAGCCCATCAGAGAGCAGGCCGAGAATATCATCCACCTGTTTACCCT
GACCAATCTGGGAGCCCCTGCCGCCTTCAAGTACTTTGACACCACCATCGACCGGAAGAGGTACACCA
GCACCAAAGAGGTGCTGGACGCCACCCTGATCCACCAGAGCATCACCGGCCTGTACGAGACACGGATC
GACCTGTCTCAGCTGGGAGGCGACaagcgacctgccgccacaaagaaggctgqacaggctaagaagaa
gaaactggactctggaggatccgactacaaagaccatgacggtgattataaagatcatgacatcgatt
acaaggatgacgatgacaagggaggatccaaggagaagagtgcttgtcctaaagatccagccaaacct
ccggccaaggcacaagttgtgggatggccaccggtgagatcataccggaagaacgtgatggtttcctg
ccaaaaatcaagcggtggcccggaggcggcggcgttcgtgaaggtatcaatggacggagcaccgtact
tgaggaaaatcgatttgaggatgtataaaggcggatctggcggctctggaggatccagcATGTCTGTG
GCTTTCGCGGCCCCGAGGCAGCGAGGCAAGGGGGAGATCACTCCCGCTGCGATTCAGAAGATGTTGGA
TGACAATAACCATCTTATTCAGTGTATAATGGACTCTCAGAATAAAGGAAAGACCTCAGAGTGTTCTC
AGTATCAGCAGATGTTGCACACAAACTTGGTATACCTTGCTACAATAGCAGATTCTAATCAAAATATG
CAGTCTCTTTTACCAGCACCACCCACACAGAATATGCCTATGGGTCCTGGAGGGATGAATCAGAGCGG
CCCTCCCCCACCTCCACGCTCTCACAACATGCCTTCAGATGGAATGGTAGGTGGGGGTCCTCCTGCAC
CGCACATGCAGAACCAGATGAACGGCCAGATGCCTGGGCCTAACCATATGCCTATGCAGGGACCTGGA
CCCAATCAACTCAATATGACAAACAGTTCCATGAATATGCCTTCAAGTAGCCATGGATCCATGGGAGG
TTACAACCATTCTGTGCCATCATCACAGAGCATGCCAGTACAGAATCAGATGACAATGAGTCAGGGAC
AACCAATGGGAAACTATGGTCCCAGACCAAATATGAGTATGCAGCCAAACCAAGGTCCAATGATGCAT
CAGCAGCCTCCTTCTCAGCAATACAATATGCCACAGGGAGGCGGACAGCATTACCAAGGACAGCAGCC
ACCTATGGGAATGATGGGTCAAGTTAACCAAGGCAATCATATGATGGGTCAGAGACAGATTCCTCCCT
ATAGACCTCCTCAACAGGGCCCACCACAGCAGTACTCAGGCCAGGAAGACTATTACGGGGACCAATAC
AGTCATGGTGGACAAGGTCCTCCAGAAGGCATGAACCAGCAATATTACCCTGATGGAAATTCACAGTA
TGGCCAACAGCAAGATGCATACCAGGGACCACCTCCACAACAGGGATATCCACCCCAGCAGCAGCAGT
ACCCAGGGCAGCAAGGTTACCCAGGACAGCAGCAGGGCTACGGTCCTTCACAGGGTGGTCCAGGTCCT
CAGTATCCTAACTACCCACAGGGACAAGGTCAGCAGTATGGAGGATATAGACCAACACAGCCTGGACC
ACCACAGCCACCCCAGCAGAGGCCTTATGGATATGACCAGGGACAGTATGGAAATTACCAGCAGTGA
Amino acid sequence fordCas9-SS18
(corresponding to SEQ ID NO: 87); capital
underlined = dCas9; capital no underline = SS18.
SEQ ID NO: 88
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLvvAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGDkrpaatkkagqakkkkidsggsdykdhdgdykdhdidykddddkggskeksacpkdpakp
pakaqvvgwppvrsyrknvmvscqkssggpeaaafvkvsmdgapylrkidlrmykggsggsggssMSV
AFAAPRQRGKGEITPAAIQKMLDDNNHLIQCIMDSQNKGKTSECSQYQQMLHTNLVYLATIADSNQNM
QSLLPAPPTQNMPMGPGGMNQSGPPPPPRSHNMPSDGMVGGGPPAPHMQNQMNGQMPGPNEMPMQGPG
PNQLNMTNSSMNMPSSSHGSMGGYNHSVPSSQSMPVQNQMTMSQGQPMGNYGPRPNMSMQPNQGPMMH
QQPPSQQYNMPQGGGQHYQGQQPPMGMMGQVNQGNHMMGQRQIPPYRPPQQGPPQQYSGQEDYYGDQY
SHGGQGPPEGMNQQYYPDGNSQYGQQQDAYQGPPPQQGYPPQQQQYPGQQGYPGQQQGYGPSQGGPGP
QYPNYPQGQGQQYGGYRPTQPGPPQPPQQRPYGYDQGQYGNYQQ*
DNA sequence fordCas9-SS18 (in backbone pNH 44): pNi228;
capital underlined = dCas9;
capital no underline = SS18.
SEQ ID NO: 89
ATGGACAAGAAGTACTCCATTGGGCTCGCCATCGGCACAAACAGCGTCGGCTGGGCCGTCATTACGGA
CGAGTACAAGGTGCCGAGCAAAAAATTCAAAGTTCTGGGCAATACCGATCGCCACAGCATAAAGAAGA
ACCTCATTGGCGCCCTCCTGTTCGACTCCGGGGAAACCGCCGAAGCCACGCGGCTCAAAAGAACAGCA
CGGCGCAGATATACCCGCAGAAAGAATCGGATCTGCTACCTGCAGGAGATCTTTAGTAATGAGATGGC
TAAGGTGGATGACTCTTTCTTCCATAGGCTGGAGGAGTCCTTTTTGGTGGAGGAGGATAAAAAGCACG
AGCGCCACCCAATCTTTGGCAATATCGTGGACGAGGTGGCGTACCATGAAAAGTACCCAACCATATAT
CATCTGAGGAAGAAGCTTGTAGACAGTACTGATAAGGCTGACTTGCGGTTGATCTATCTCGCGCTGGC
GCATATGATCAAATTTCGGGGACACTTCCTCATCGAGGGGGACCTGAACCCAGACAACAGCGATGTCG
ACAAACTCTTTATCCAACTGGTTCAGACTTACAATCAGCTTTTCGAAGAGAACCCGATCAACGCATCC
GGAGTTGACGCCAAAGCAATCCTGAGCGCTAGGCTGTCCAAATCCCGGCGGCTCGAAAACCTCATCGC
ACAGCTCCCTGGGGAGAAGAAGAACGGCCTGTTTGGTAATCTTATCGCCCTGTCACTCGGGCTGACCC
CCAACTTTAAATCTAACTTCGACCTGGCCGAAGATGCCAAGCTTCAACTGAGCAAAGACACCTACGAT
GATGATCTCGACAATCTGCTGGCCCAGATCGGCGACCAGTACGCAGACCTTTTTTTGGCGGCAAAGAA
CCTGTCAGACGCCATTCTGCTGAGTGATATTCTGCGAGTGAACACGGAGATCACCAAAGCTCCGCTGA
GCGCTAGTATGATCAAGCGCTATGATGAGCACCACCAAGACTTGACTTTGCTGAAGGCCCTTGTCAGA
CAGCAACTGCCTGAGAAGTACAAGGAAATTTTCTTCGATCAGTCTAAAAATGGCTACGCCGGATACAT
TGACGGCGGAGCAAGCCAGGAGGAATTTTACAAATTTATTAAGCCCATCTTGGAAAAAATGGACGGCA
CCGAGGAGCTGCTGGTAAAGCTTAACAGAGAAGATCTGTTGCGCAAACAGCGCACTTTCGACAATGGA
AGCATCCCCCACCAGATTCACCTGGGCGAACTGCACGCTATCCTCAGGCGGCAAGAGGATTTCTACCC
CTTTTTGAAAGATAACAGGGAAAAGATTGAGAAAATCCTCACATTTCGGATACCCTACTATGTAGGCC
CCCTCGCCCGGGGAAATTCCAGATTCGCGTGGATGACTCGCAAATCAGAAGAGACCATCACTCCCTGG
AACTTCGAGGAAGTCGTGGATAAGGGGGCCTCTGCCCAGTCCTTCATCGAAAGGATGACTAACTTTGA
TAAAAATCTGCCTAACGAAAAGGTGCTTCCTAAACACTCTCTGCTGTACGAGTACTTCACAGTTTATA
ACGAGCTCACCAAGGTCAAATACGTCACAGAAGGGATGAGAAAGCCAGCATTCCTGTCTGGAGAGCAG
AAGAAAGCTATCGTGGACCTCCTCTTCAAGACGAACCGGAAAGTTACCGTGAAACAGCTCAAAGAAGA
CTATTTCAAAAAGATTGAATGTTTCGACTCTGTTGAAATCAGCGGAGTGGAGGATCGCTTCAACGCAT
CCCTGGGAACGTATCACGATCTCCTGAAAATCATTAAAGACAAGGACTTCCTGGACAATGAGGAGAAC
GAGGACATTCTTGAGGACATTGTCCTCACCCTTACGTTGTTTGAAGATAGGGAGATGATTGAAGAACG
CTTGAAAACTTACGCTCATCTCTTCGACGACAAAGTCATGAAACAGCTCAAGAGGCGCCGATATACAG
GATGGGGGCGGCTGTCAAGAAAACTGATCAATGGGATCCGAGACAAGCAGAGTGGAAAGACAATCCTG
GATTTTCTTAAGTCCGATGGATTTGCCAACCGGAACTTCATGCAGTTGATCCATGATGACTCTCTCAC
CTTTAAGGAGGACATCCAGAAAGCACAAGTTTCTGGCCAGGGGGACAGTCTTCACGAGCACATCGCTA
ATCTTGCAGGTAGCCCAGCTATCAAAAAGGGAATACTGCAGACCGTTAAGGTCGTGGATGAACTCGTC
AAAGTAATGGGAAGGCATAAGCCCGAGAATATCGTTATCGAGATGGCCCGAGAGAACCAAACTACCCA
GAAGGGACAGAAGAACAGTAGGGAAAGGATGAAGAGGATTGAAGAGGGTATAAAAGAACTGGGGTCCC
AAATCCTTAAGGAACACCCAGTTGAAAACACCCAGCTTCAGAATGAGAAGCTCTACCTGTACTACCTG
CAGAACGGCAGGGACATGTACGTGGATCAGGAACTGGACATCAATCGuCTCTCCGACTACGACGTGGA
TGCCATCGTGCCCCAGTCTTTTCTCAAAGATGATTCTATTGATAATAAAGTGTTGACAAGATCCGATA
AAAATAGAGGGAAGAGTGATAACGTCCCCTCAGAAGAAGTTGTCAAGAAAATGAAAAATTATTGGCGG
CAGCTGCTGAACGCCAAACTGATCACACAACGGAAGTTCGATAATCTGACTAAGGCTGAACGAGGTGG
CCTGTCTGAGTTGGATAAAGCCGGCTTCATCAAAAGGCAGCTTGTTGAGACACGCCAGATCACCAAGC
ACGTGGCCCAAATTCTCGATTCACGCATGAACACCAAGTACGATGAAAATGACAAACTGATTCGAGAG
GTGAAAGTTATTACTCTGAAGTCTAAGCTGGTCTCAGATTTGAGAAAGGACTTTGAGTTTTATAAGGT
GAGAGAGATCAACAATTACCACCATGCGCATGATGCCTACCTGAATGCAGTGGTAGGCACTGCACTTA
TCAAAAAATATCCCAAGCTTGAATCTGAATTTGTTTACGGAGACTATAAAGTGTACGATGTTAGGAAA
ATGATCGCAAAGTCTGAGCAGGAAATAGGCAAGGCCACCGCTAAGTACTTCTTTTACAGCAATATTAT
GAATTTTTTCAAGACCGAGATTACACTGGCCAATGGAGAGATTCGGAAGCGACCACTTATCGAAACAA
ACGGAGAAACAGGAGAAATCGTGTGGGACAAGGGTAGGGATTTCGCGACAGTCCGGAAGGTCCTGTCC
ATGCCGCAGGTGAACATCGTTAAAAAGACCGAAGTACAGACCGGAGGCTTCTCCAAGGAAAGTATCCT
CCCGAAAAGGAACAGCGACAAGCTGATCGCACGCAAAAAAGATTGGGACCCCAAGAAATACGGCGGAT
TCGATTCTCCTACAGTCGCTTACAGTGTACTGGTTGTGGCCAAAGTGGAGAAAGGGAAGTCTAAAAAA
CTCAAAAGCGTCAAGGAACTGCTGGGCATCACAATCATGGAGCGATCAAGCTTCGAAAAAAACCCCAT
CGACTTTCTCGAGGCGAAAGGATATAAAGAGGTCAAAAAAGACCTCATCATTAAGCTTCCCAAGTACT
CTCTCTTTGAGCTTGAAAACGGCCGGAAACGAATGCTCGCTAGTGCGGGCGAGCTGCAGAAAGGTAAC
GAGCTGGCACTGCCCTCTAAATACGTTAATTTCTTGTATCTGGCCAGCCACTATGAAAAGCTCAAAGG
GTCTCCCGAAGATAATGAGCAGAAGCAGCTGTTCGTGGAACAACACAAACACTACCTTGATGAGATCA
TCGAGCAAATAAGCGAATTCTCCAAAAGAGTGATCCTCGCCGACGCTAACCTCGATAAGGTGCTTTCT
GCTTACAATAAGCACAGGGATAAGCCCATCAGGGAGCAGGCAGAAAACATTATCCACTTGTTTACTCT
GACCAACTTGGGCGCGCCTGCAGCCTTCAAGTACTTCGACACCACCATAGACAGAAAGCGGTACACCT
CTACAAAGGAGGTCCTGGACGCCACACTGATTCATCAGTCAATTACGGGGCTCTATGAAACAAGAATC
GACCTCTCTCAGCTCGGTGGAGACAGCAGGGCTGACcccaagaagaagaggaaggtggctagCATGTC
TGTGGCTTTCGCGGCCCCGAGGCAGCGAGGCAAGGGGGAGATCACTCCCGCTGCGATTCAGAAGATGT
TGGATGACAATAACCATCTTATTCAGTGTATAATGGACTCTCAGAATAAAGGAAAGACCTCAGAGTGT
TCTCAGTATCAGCAGATGTTGCACACAAACTTGGTATACCTTGCTACAATAGCAGATTCTAATCAAAA
TATGCAGTCTCTTTTACCAGCACCACCCACACAGAATATGCCTATGGGTCCTGGAGGGATGAATCAGA
GCGGCCCTCCCCCACCTCCACGCTCTCACAACATGCCTTCAGATGGAATGGTAGGTGGGGGTCCTCCT
GCACCGCACATGCAGAACCAGATGAACGGCCAGATGCCTGGGCCTAACCATATGCCTATGCAGGGACC
TGGACCCAATCAACTCAATATGACAAACAGTTCCATGAATATGCCTTCAAGTAGCCATGGATCCATGG
GAGGTTAGAACCATTCTGTGCCATCATCACAGAGCATGCCAGTACAGAATCAGATGACAATGAGTCAG
GGACAACCAATGGGAAACTATGGTCCCAGACCAAATATGAGTATGCAGCCAAACCAAGGTCCAATGAT
GCATCAGCAGCCTCCTTCTCAGCAATACAATATGCCACAGGGAGGCGGACAGCATTACCAAGGACAGC
AGCCACCTATGGGAATGATGGGTCAAGTTAACCAAGGCAATCATATGATGGGTCAGAGACAGATTCCT
CCCTATAGACCTCCTCAACAGGGCCCACCACAGCAGTACTCAGGCCAGGAAGACTATTACGGGGACCA
ATACAGTCATGGTGGACAAGGTCCTCCAGAAGGCATGAACCAGCAATATTACCCTGATGGAAATTCAC
AGTATGGCCAACAGCAAGATGCATACCAGGGACCACCTCCACAACAGGGATATCCACCCCAGCAGCAG
CAGTACCCAGGGCAGCAAGGTTACCCAGGACAGCAGCAGGGCTACGGTCCTTCACAGGGTGGTCCAGG
TCCTCAGTATCCTAACTACCCACAGGGACAAGGTCAGCAGTATGGAGGATATAGACCAACACAGCCTG
GACCACCACAGCCACCCCAGCAGAGGCCTTATGGATATGACCAGGGACAGTATGGAAATTACCAGCAG
Amino acid sequence fordCas9-SS18
(corresponding to SEQ ID NO: 89); capital
underllned = dCas9; capital no underline = SS18.
SEQ ID NO: 90
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFTERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAvvGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNTVKKTEVQTGGFSKESILPKRNSDKLIARKKDWDPKKYGGFDSPTVAYSVLVVARVEKGKSKK
LKSVKELLGITIMERSSFEKNPIDFLEAKGYKEVKKDLIIKLPKYSLFELENGRKRMLASAGELQKGN
ELALPSKYVNFLYLASHYEKTKGSPEDNFOKOLFVFOHKHYLDETTEQJSEFSKRVILADANLDKVLS
AYNKHRDKPIREQAENIIHLFTLTNLGAPAAFKYFDTTIDRKRYTSTKEVLDATLIHQSITGLYETRI
DLSQLGGDSRADpkkkrkvasMSVAFAAPRQRGKGEITPAAIQKMLDDNNHLIQCIMDSQNKGKTSEC
SQYQQMLHTNLVYLATIADSNQNMQSLLPAPPTQNMPMGPGGMNQSGPPPPPRSHNMPSDGMVGGGPP
APHMQNQMNGQMPGPNHMPMQGPGPNQLNMTNSSMNMPSSSHGSMGGYNHSVPSSQSMPVQNQMTMSQ
GOPMGNYGPRPNMSMOPNQGPMMHQQPPSOQYNMPQGGGQHYQGOQPPMGMMGQVNQGNHMMGQROIP
PYRPPQQGPPQQYSGQEDYYGDQYSHGGQGPPEGMNQQYYPDGNSQYGQQQDAYQGPPPQQGYPPQQQ
QYPGQQGYPGQQQGYGPSQGGPGPQYPNYPQGQGQQYGGYRPTQPGPPQPPQQRPYGYDQGQYGNYQQ
Amino acid sequence of VP64
SEQ ID NO: 91
RADALDDFDLDMLGSDALDDFDLDHLGSDALDDFDLDMLGSDALDDFDLDM
DNA sequence of VP64
SEQ ID NO: 92
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatg
Polypeptide sequence of Tet1CD
SEQ ID NO: 93
LPTCSCLDRVIQKDKGPYYTHLGAGPSVAAVREIMENRYGQKGNAIRIEIVVYTGKEGKSSHGCPIAK
WVLRRSSDEEKVLCLVRQRTGHHCPTAVMVVLIMVWDGIPLPMADRLYTELTENLKSYNGHPTDRRCT
LNENRTCTCQGIDPETCGASFSFGCSWSMYFNGCKFGRSPSPRRFRIDPSSPLHEKNLEDNLQSLATR
LAPIYKQYAPVAYQNQVEYENVARECRLGSKEGRPFSGVTACLDFCAHPHRDIHNMNNGSTVVCTLTR
EDNRSLGVIPODEQLHVLPLYKLSDTDEFGSKEGMEAKIKSGAIEVLAPRRKKRTCFTQPVPRSGKKR
AAMMTEVLAHKIRAVEKKPIPRIKRKNNSTTTNNSKPSSLPTLGSNTETVQPEVKSETEPHFILKSSD
NTKTYSLMPSAPHPVKEASPGFSWSPKTASATPAPLKNDATASCGFSERSSTPHCTMPSGRLSGANAA
AADGPGISQLGEVAPLPTLSAPVMEPLINSEPSTGVTEPLTPHQPNHQPSFLTSPQDLASSPMEEDEQ
HSEADEPPSDEPLSDDPLSPAEEKLPHIDEYWSDSEHIFLDANIGGVAIAPAHGSVLIECARRELHAT
TPVEHPNRNHPTRLSLVFYQHKNLNKPQHGFELNKIKFEAKEAKNKKMKASEQKDQAANEGPEQSSEV
NELNQIPSHKALTLTHDNVVTVSPYALTHVAGPYNHWV
Polynucleotide sequence of Tet1CD
SEQ ID NO: 94
CTGCCCACCTGCAGCTGTCTTGATCGAGTTATACAAAAAGACAAAGGCCCATATTATACACACCTTGG
GGCAGGACCAAGTGTTGCTGCTGTCAGGGAAATCATGGAGAATAGGTATGGTCAAAAAGGAAACGCAA
TAAGGATAGAAATAGTAGTGTACACCGGTAAAGAAGGGAAAAGCTCTCATGGGTGTCCAATTGCTAAG
TGGGTTTTAAGAAGAAGCAGTGATGAAGAAAAAGTTCTTTGTTTGGTCCGGCAGCGTACAGGCCACCA
CTGTCCAACTGCTGTGATGGTGGTGCTCATCATGGTGTGGGATGGCATCCCTCTTCCAATGGCCGACC
GGCTATACACAGAGCTCACAGAGAATCTAAAGTCATACAATGGGCACCCTACCGACAGAAGATGCACC
CTCAATGAAAATCGTACCTGTACATGTCAAGGAATTGATCCAGAGACTTGTGGAGCTTCATTCTCTTT
TGGCTGTTCATGGAGTATGTACTTTAATGGCTGTAAGTTTGGTAGAAGCCCAAGCCCCAGAAGATTTA
GAATTGATCCAAGCTCTCCCTTAGATGAAAAAAACCTTGAAGATAACTTACAGAGTTTGGCTAGACGA
TTAGCTCCAATTTATAAGCAGTATGCTCCAGTAGCTTACCAAAATCAGGTGGAATATGAAAATGTTGC
CCGAGAATGTCGGCTTGGCAGCAAGGAAGGTCGACCCTTCTCTGGGGTCACTGCTTGCCTGGACTTCT
GTGCTCATCCCCACAGGGACATTCACAACATGAATAATGGAAGCACTGTGGTTTGTACCTTAACTCGA
GAAGATAACCGCTCTTTGGGTGTTATTCCTCAAGATGAGCAGCTCCATGTGCTACCTCTTTATAAGCT
TTCAGACACAGATGAGTTTGGCTCCAAGGAAGGAATGGAAGCCAAGATCAAATCTGGGGCCATCGAGG
TCCTGGCACCCCGCCGCAAAAAAAGAACGTGTTTCACTCAGCCTGTTCCCCGTTCTGGAAAGAAGAGG
GCTGCGATGATGACAGAGGTTCTTGCACATAAGATAAGGGCAGTGGAAAAGAAACCTATTCCCCGAAT
CAAGCGGAAGAATAACTCAACAACAACAAACAACAGTAAGCCTTCGTCACTGCCAACCTTAGGGAGTA
ACACTGAGACCGTGCAACCTGAAGTAAAAAGTGAAACCGAACCCCATTTTATCTTAAAAAGTTCAGAC
AACACTAAAACTTATTCGCTGATGCCATCCGCTCCTCACCCAGTGAAAGAGGCATCTCCAGGCTTCTC
CTGGTCCCCGAAGACTGCTTCAGCCACACCAGCTCCACTGAAGAATGACGCAACAGCCTCATGCGGGT
TTTCAGAAAGAAGCAGCACTCCCCACTGTACGATGCCTTCGGGAAGACTCAGTGGTGCCAATGCTGCA
GCTGCTGATGGCCCTGGCATTTCACAGCTTGGCGAAGTGGCTCCTCTCCCCACCCTGTCTGCTCCTGT
GATGGAGCCCCTCATTAATTCTGAGCCTTCCACTGGTGTGACTGAGCCGCTAACGCCTCATCAGCCAA
ACCACCAGCCCTCCTTCCTCACCTCTCCTCAAGACCTTGCCTCTTCTCCAATGGAAGAAGATGAGCAG
CATTCTGAAGCAGATGAGCCTCCATCAGACGAACCCCTATCTGATGACCCCCTGTCACCTGCTGAGGA
GAAATTGCCCCACATTGATGAGTATTGGTCAGACAGTGAGCACATCTTTTTGGATGCAAATATTGGTG
GGGTGGCCATCGCACCTGCTCACGGCTCGGTTTTGATTGAGTGTGCCCGGCGAGAGCTGCACGCTACC
ACTCCTGTTGAGCACCCCAACCGTAATCATCCAACCCGCCTCTCCCTTGTCTTTTACCAGCACAAAAA
CCTAAATAAGCCCCAACATGGTTTTGAACTAAACAAGATTAAGTTTGAGGCTAAAGAAGCTAAGAATA
AGAAAATGAAGGCCTCAGAGCAAAAAGACCAGGCAGCTAATGAAGGTCCAGAACAGTCCTCTGAAGTA
AATGAATTGAACCAAATTCCTTCTCATAAAGCATTAACATTAACCCATGACAATGTTGTCACCGTGTC
CCCTTATGCTCTCACACACGTTGCGGGGCCCTATAACCATTGGGTC

1. A fusion protein comprising at least two heterologous polypeptide domains, wherein the first polypeptide domain comprises a DNA binding protein and the second polypeptide domain comprises a modulator of chromatin structure.

2. The fusion protein of claim 1, wherein the fusion protein further comprises a third polypeptide domain.

3. The fusion protein of any one of the preceding claims, wherein the first polypeptide domain comprises a CRISPR-associated (Cas) protein, a TALE, or a zinc finger protein.

4. The fusion protein of claim 3, wherein the Cas protein comprises at east one amino acid mutation that eliminates nuclease activity of the Cas protein.

5. The fusion protein of claim 3 or 4, wherein the Cas protein comprises a Cas9 protein.

6. The fusion protein of claim 5, wherein the Cas9 protein is nuclease-deficient dCas9 and comprises a polypeptide having at least 75% sequence identity to SEQ ID NO: 20 or 21 or is encoded by a polynucleotide comprising a sequence having at least 75% identity to SEQ ID NO: 22 or 23.

7. The fusion protein of any one of the preceding claims, wherein the modulator of chromatin structure comprises a nucleosome rearranging protein.

8. The fusion protein of any one of the preceding claims, wherein the modulator of chromatin structure comprises the SSI 8 subunit of the BAF chromatin remodeling complex or a fragment thereof or a variant thereof.

9. The fusion protein of claim 8, wherein the SSI 8 subunit comprises a polypeptide having at least 75% sequence identity to SEQ ID NO: 37.

10. The fusion protein of any one of claims 2-9, wherein the third polypeptide domain comprises a transcriptional activator domain.

11. The fusion protein of claim 10, wherein the transcriptional activator domain comprises VP64, VPH, VPR, p65, TET1, or p300, or a combination thereof or a fragment thereof or a variant thereof.

12. The fusion protein of claim 11, wherein the VP64 comprises a polypeptide having at least 75% sequence identity to SEQ ID NO: 91.

13. The fusion protein of claim 11, wherein the TET1 comprises a polypeptide having at least 75% sequence identity to SEQ ID NO: 93.

14. The fusion protein of claim 11, wherein the VPH comprises a polypeptide having at least 75% sequence identity to SEQ ID NO: 39.

15. The fusion protein of claim 11, wherein the VPR comprises a polypeptide having at least 75% sequence identity to SEQ ID NO: 41.

16. The fusion protein of claim 11, wherein the p300 comprises a polypeptide having at least 75% sequence identity to SEQ ID NO: 33 or 34.

17. The fusion protein of any one of claims 1-16, wherein the fusion protein comprises one or more second polypeptide domain(s).

18. The fusion protein of claim 17, wherein the one or more second polypeptide domain(s) is fused to the C-terminus or the N-terminus of the first polypeptide domain, or a combination thereof.

19. The fusion protein of claim 18, wherein the N-terminus of the second polypeptide is operably linked to the C-terminus of the first polypeptide domain, or wherein the C-terminus of the second polypeptide is operably linked to the N-terminus of the first polypeptide domain.

20. The fusion protein of any one of claims 2-19, wherein the fusion protein comprises one or more third polypeptide domain(s).

21. The fusion protein of claim 20, wherein the one or more third polypeptide domain is fused to the C-terminus or the N-terminus of the first polypeptide domain, or a combination thereof.

22. The fusion protein of claim 21, wherein the N-terminus of the third polypeptide is operably linked to the C-terminus of the first polypeptide domain, or wherein the C-terminus of the third polypeptide is operably linked to the N-terminus of the first polypeptide domain.

23. The fusion protein of any one of claims 2-22, wherein the first polypeptide domain comprises dCas9, wherein the second polypeptide domain comprises SS18, and wherein the third polypeptide domain comprises VPH.

24. The fusion protein of claim 23, wherein the fusion protein comprises VPH-dCas9-SS18 or SS18-dCas9-VPH or variants thereof.

25. The fusion protein of claim 24, wherein the fusion protein comprises a polypeptide having at least 75% sequence identity to SEQ ID NO: 64 or 66.

26. The fusion protein of any one of claims 2-22, wherein the first polypeptide domain comprises dCas9, wherein the second polypeptide domain comprises SS18, and wherein the third polypeptide domain comprises VPR.

27. The fusion protein of claim 26, wherein the fusion protein comprises VPR-dCas9-SS18 or SS18-dCas9-VPR or variants thereof.

28. The fusion protein of any one of claims 2-22, wherein the first polypeptide domain comprises dCas9, wherein the second polypeptide domain comprises SS18, and wherein the third polypeptide domain comprises p300.

29. The fusion protein of claim 28, wherein the fusion protein comprises p300-dCas9-SS18 or SS18-dCas9-p300 or variants thereof.

30. The fusion protein of any one of claims 2-22, wherein the first polypeptide domain comprises dCas9, wherein the second polypeptide domain comprises SS18, and wherein the third polypeptide domain comprises VP64.

31. The fusion protein of claim 30, wherein the fusion protein comprises VP64-dCas9-SS18 or SS18-dCas9-VP64 or variants thereof.

32. The fusion protein of any one of the preceding claims, wherein the fusion protein activates transcription of a target gene.

33. The fusion protein of any one of the preceding claims, wherein the fusion protein increases the level of mRNA expression of a target gene in a cell containing the fusion protein relative to a control.

34. The fusion protein of claim 33, wherein the level of mRNA expression of the target gene is increased at least 5-fold, at least 50-fold, at least 100-fold, at least 1,000-fold, at least 10,000-fold, or at least 20,000-fold relative to a control.

35. The fusion protein of claim 33 or 34, wherein the level of mRNA expression of the target gene is increased by 5-fold to 10,000-fold, 5-fold to 30,000-fold, 5-fold to 50,000-fold, 5-fold to 100,000-fold, 10,000-fold to 30,000-fold, 20,000-fold to 30,000-fold, 15,000-fold to 25,000-fold, 1,000-fold to 50,000-fold, or 1,000-fold to 100,000-fold relative to a control.

36. The fusion protein of any one of claims 33-35, wherein the control is the level of mRNA expression of the target gene in a cell not containing the fusion protein.

37. The fusion protein of any one of claims 32-36, wherein the target gene is gamma globin genes 1 and 2 (HBG1/2).

38. A DNA Targeting System comprising:
(a) the fusion protein of any one of claims 1-37, wherein the first polypeptide domain comprises a zinc finger protein or a TALE; or
(b) a gRNA and the fusion protein of any one of claims 1-37, wherein the first polypeptide domain comprises a Cas protein, and wherein the gRNA targets a target gene.

39. The DNA Targeting System of claim 38, wherein gRNA targets a regulatory region of the target gene.

40. The DNA Targeting System of claim 39, wherein the regulatory region is a promoter sequence of the target gene.

41. A DNA Targeting System comprising a gRNA that recruits a modulator of chromatin structure to a target sequence.

42. The DNA Targeting System of claim 41, wherein the modulator of chromatin structure comprises the SS18 subunit of the BAF chromatin remodeling complex.

43. The DNA Targeting System of any one of claims 38-42, wherein the gRNA is encoded by or binds to a target sequence selected from SEQ ID NOs: 43-48, a complement thereof, a truncation thereof, or a variant thereof, or wherein the gRNA is encoded by or binds to a target sequence having at least 70% sequence identity to a sequence selected from SEQ ID NOs: 43-48, a complement thereof, a truncation thereof, or a variant thereof.

44. The DNA Targeting System of any one of claims 38-43, wherein the gRNA comprises a polynucleotide sequence selected from SEQ ID NOs: 49-54, a complement thereof, a truncation thereof, or a variant thereof, or wherein the gRNA comprises a polynucleotide having at least 70% sequence identity to a sequence selected from SEQ ID NOs: 49-54, a complement thereof, a truncation thereof, or a variant thereof.

45. A method of increasing expression of a target gene in a cell, the method comprising contacting the cell with the fusion protein of any one of claims 1-37 or the DNA Targeting system of any one of claims 38-44.

46. The method of claim 45, wherein the target gene is gamma globin genes 1 and 2 (HBG1/2).

47. A gRNA encoded by or binding to a target sequence selected from SEQ ID NOs: 43-48, a complement thereof, a truncation thereof, or a variant thereof, or comprising a polynucleotide sequence selected from SEQ ID NOs: 49-54, a complement thereof, a truncation thereof, or a variant thereof.
